(19) United States
(12) Patent Application Publication
(10) Pub. No.: US 2024/0261389 A1
(43) Pub. Date: Aug. 8, 2024

(54) AMINO ACIDS, NUCLEOTIDES AND VECTORS EXPRESSING THE SAME AND USES THEREOF IN PREVENTING SARBECOVIRUS INFECTION

(71) Applicant: National University of Singapore, Singapore (SG)

(72) Inventors: Linfa Wang, Singapore (SG); Wan Ni Chia, Singapore (SG); Chee Wah Tan, Singapore (SG); Feng Zhu, Singapore (SG)

(21) Appl. No.: 18/561,154

(22) PCT Filed: May 15, 2022
(86) PCT No.: PCT/SG2022/050322

§ 371 Date: Nov. 15, 2023

(30) Foreign Application Priority Data
May 15, 2021 (SG) 10202105099S

(51) Int. Cl. A61K 39/215; A61K 39/00; A61P 31/14; C07K 14/005; C12N 7/00; C12N 15/86

(52) U.S. Cl. CPC A61K 39/215; A61P 31/14; C07K 14/005; C12N 7/00; C12N 15/86; A61K 2039/53; A61K 2039/55566; A61K 2039/55572; C12N 2710/24022; C12N 2710/24043; C12N 2750/14122; C12N 2750/14143; C12N 2760/18422; C12N 2760/18443; C12N 2760/20222; C12N 2760/20243; C12N 2770/20034

(57) ABSTRACT

Disclosed herein is a method of making an amino acid construct for the treatment and/or prevention of sarbecovirus infections, comprising: a. comparing amino acid sequences from at least two different sarbecovirus spike proteins or fragments thereof; b. identifying identical amino acids in the sequences from the at least two different sarbecovirus spike proteins or fragments thereof; c. removing any different amino acids from the sequences of the at least two different sarbecovirus spike proteins or fragments thereof to identify a unique amino acid sequence; and d. forming the amino acid construct of the unique amino acid sequence wherein the amino acid construct has at least 90% sequence identity to the at least two different sarbecovirus spike proteins or fragments thereof. Also disclosed are amino acid sequences generated using the method of the invention.

### REFERENCE TO RELATED APPLICATIONS

This application claims the priority to Singapore patent application No. 10202105099S, filed 15 May 2021, the contents of which are incorporated herein by reference.

### TECHNICAL FIELD

The present invention generally relates to proteins and their use in the treatment and/or prevention of Severe Acute Respiratory Syndrome (SARS)-related coronavirus infections, particularly sarbecovirus infections.

### BACKGROUND

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

Since 2002, we had three major human infectious diseases caused by coronaviruses (CoVs), SARS coronavirus (SARS-CoV) in 2002-2003 (Peiris et al. Nat Med 2004, 10:S88-97), Middle East Respiratory Syndrome coronavirus (MERS-CoV) since 2012 (Zaki et al. N Engl J Med 2012, 367:1814-1820) and the ongoing COVID-19 or SARS coronavirus 2 (SARS-CoV-2) (Wang et al. Lancet 2020, 395:470-473). All of these diseases have caused devastating economic and human losses globally. For SARS-CoV and MERS-CoV, we still don't have a licensed vaccine to protect us from future infections. For SARS-CoV-2, the unprecedented speed of vaccine development has resulted in many licensed vaccines for human use (Fauci, Science 2021, 372:109).

However, the recent emergence of SARS-CoV-2 variants such as the SARS-CoV-2 B.1.1.7/201/501Y.V1 detected in the UK; the South African SARS-CoV-2 B.1.351/20H/501Y.V2, the Brazil SARS-CoV-2 P.1/20J/501Y.V3/B.1.1.248, and the U.S. SARS-CoV-2 B.1.1427/B.1.429 (Mascola et al. JAMA 2021, 325:1261-1262; Zhang et al. JAMA 2021, 325(13):1324-1326) and the observed reduction of immune protection against new variants from vaccines based on the original virus strain raised a new challenge on the need for a broad-spectrum protection against all known and future SARS-CoV-2 variants.

Furthermore, there are many more coronaviruses circulating in wildlife reservoirs such as the SC2r-CoV RaTG13 in bats and the SC2r-CoV GX-P5L in pangolins (Wang et al. Curr Opin Virol 2019, 34:79-89; Zhou et al. Nature 2020, 579:270-273; Lam et al. Nature 2020, 583:282-285).

In addition, there is a high chance of future outbreaks (SARS3, SARS4, etc.) caused by different but related coronaviruses (Calistri et al. Microorganisms 2021, 9).

The current classification of coronaviruses is shown in FIG. 1. Four genera, alpha, beta, delta and gamma have been identified. The most transmissible coronaviruses for human infections are from the sarbecoviruses group. It was also found that all sarbecoviruses use the angiotensin-converting enzyme 2 (ACE2) as an entry receptor to human host cells.

Accordingly, there exists a need for prophylactic or therapeutic vaccine for the treatment and/or prevention of current and any future infections caused by sarbecoviruses.

### SUMMARY

Amino acid constructs, nucleotides, vectors and immunogenic composition containing or expressing the amino acid sequences used in preventing and/or treating a sarbecovirus infection are envisioned.

According to an aspect of the present disclosure, there is provided an amino acid construct comprising any one of amino acid sequences selected from the group comprising SEQ ID Nos. 10 to 22 164 and 165, the amino acid construct having at least 90% sequence identity to at least two different sarbecovirus Spike proteins, or a fragment thereof.

According to another aspect there is a nucleic acid encoding the amino acid construct as described herein above.

According to another aspect there is an immunogenic composition comprising the amino acid construct as described herein above or the nucleic acid as described herein above.

According to another aspect there is a viral vector comprising the nucleic acid as described herein above.

According to another aspect there is an amino acid construct as described herein above, a nucleic acid as described herein above, an immunogenic composition as described herein above, a viral vector as described herein above or vaccine formulations suitable for use in the treatment or prevention of sarbecovirus infections.

According to another aspect there is a use of an amino acid construct as described herein above, a nucleic acid as described herein above, an immunogenic composition as described herein above, a viral vector as described herein above in the manufacture of a medicament for the treatment or prevention of sarbecovirus infections.

According to another aspect there is a method for treating and/or preventing an infection caused by sarbecoviruses comprising administering a vaccine molecule comprising an amino acid construct as described herein above, a nucleic acid as described herein above, an immunogenic composition as described herein above, or a viral vector as described herein above to a subject.

According to another aspect there is method of making an amino acid construct as described herein above, comprising: a) comparing amino acid sequences from at least two different sarbecovirus spike proteins or a fragment thereof; b) identifying identical amino acid in the sequences from the at least two different sarbecovirus spike proteins or fragments thereof; c) removing any different amino acids from the sequences of the at least two different sarbecovirus spike proteins or fragments thereof to identify a unique amino acid sequence; and d) forming the amino construct of the unique amino acid sequence wherein the amino construct has at least 90% sequence identity to the at least two different sarbecovirus Spike proteins, or a fragment thereof.

Other aspects and features will become apparent to those of ordinary skill in the art upon review of the following description of specific embodiments in conjunction with the accompanying figures.

### BRIEF DESCRIPTION OF THE DRAWINGS

In the figures, which illustrate, by way of example only, embodiments of the present invention,

FIG. 1 illustrates the family tree of four known coronavirus genera;

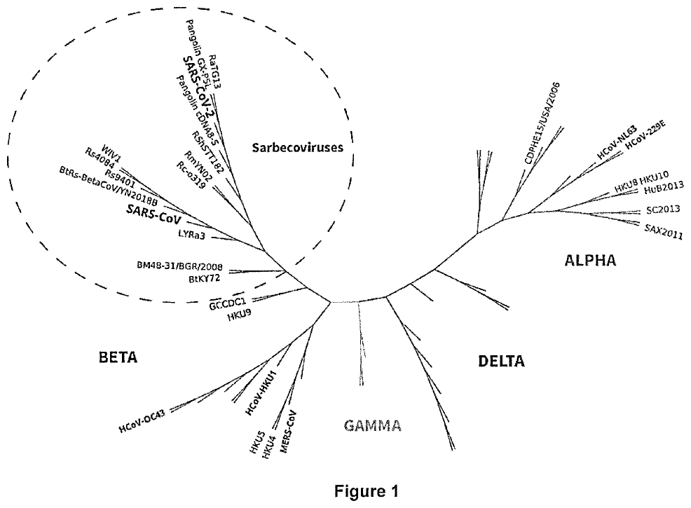

FIG. 2 illustrates the consensus groups established taking into consideration of phylogenetic relationship and ACE2 receptor usages;

FIG. 3 illustrates the design method to generate amino acid consensus sequences;

FIG. 4 illustrates a surrogate virus neutralization test (sVNT) which allowed a rapid and multiplex determination of Nabs;

FIG. 5 illustrates a A) multiplex sVNT on the Luminex platform B) showing that six RBD proteins are able to bind the hACE2 receptor molecule in the following order (from high to low affinity): SARS-CoV-2 B.1.351>SARS-CoV-2 B.1.1.7=SC2r-CoV GX-P5L (pangolin)>SARS-CoV-2>SARS-CoV>SC2r-CoV RaTG13 (bat);

FIG. 6 illustrates a multiplex sVNT on six different RBDs (from left to right: SARS-CoV-2 WT, B.1.1.7, B.1.351; Bat virus RaTG13; Pangolin virus GX-P5L; SARS-CoV). A) SARS patient (N=11); B) COVID-19 patient (N=40); C) Healthy-vaccinated (N=20). Serum samples were taken two weeks after second dose; D) SARS-vaccinated (N=9). Sera from SARS survivors taken 21-62 days after the first vaccination. All serum samples were tested at a single dilution of 1:20. A cut-off of 30% was set as previously determined.

FIG. 7 illustrates a titration of neutralizing antibody levels (NT50) in different groups against six sarbecoviruses. Serum samples were tested at dilutions from 1:20 to 1:20480 by a 4-fold serial titration.

FIG. 8 illustrates a confirmation of boosting pan-sarbecovirus cross-neutralizing antibodies in the SARS-vaccinated group. FIG. 8A shows the pan-sarbecovirus neutralization of mAb 5B7D7 against SARS-CoV-2 variants of concern, bat SC2r-CoV RaTG13, Pangolin SC2r-CoV GX-P5L and SARS-CoV measured by the multiplex sVNT. FIG. 8B illustrates an inhibition of 5B7D7 binding to different RBDs by the four different panels of sera.

FIG. 9 illustrates neutralization patterns from rabbit hyper immune sera targeting different beta coronavirus RBD proteins.

FIG. 10 illustrates challenge with 20 RBD of different sarbecovirus after vaccination with either 2 doses of 25 μg protein (SEQ ID No. 165, a modified version of SEQ ID No. 13 with foldon and linker sequence) and sigma adjuvant or one dose of 25 μg protein (SEQ ID No. 165) and sigma adjuvant followed by one dose of Sinovac vaccine.

FIG. 11 illustrates challenge with 20 RBD of different sarbecovirus after vaccination with either 2 doses of 25 μg protein (SEQ ID No. 165, a modified version of SEQ ID No. 13 with foldon and linker sequence) and sigma adjuvant or one dose of 25 μg protein (SEQ ID No. 165, a modified version of SEQ ID No. 13 with foldon and linker sequence) and sigma adjuvant followed by one dose of Sinovac vaccine.

FIG. 12 illustrates Separate dosage regimens were compared: the first being 3 doses of the Pfizer BioNTech vaccine; the second being 2 doses of the Pfizer BioNTech vaccine followed by a dose of 25 μg of the protein listed in SEQ ID No. 165 (a modified version of SEQ ID No. 13 with foldon and linker sequence) administered with Sigma adjuvant; the third being 2 doses of the Pfizer BioNTech vaccine followed by a dose of 1 μg of the protein listed in SEQ ID No. 165, a modified version of SEQ ID No. 13 with foldon and linker sequence; the fourth being 2 doses of the Pfizer BioNTech vaccine followed by a dose of saline; and the final being 3 doses of saline.

FIG. 13 illustrates Separate dosage regimens were compared: the first being 3 doses of the Moderna vaccine; the second being 2 doses of the Moderna vaccine followed by a dose of 25 μg of the protein listed in SEQ ID No. 165 (a modified version of SEQ ID No. 13 with foldon and linker sequence) administered with Sigma adjuvant; the third being 2 doses of the Moderna vaccine followed by a dose of 1 μg of the protein listed in SEQ ID No. 165 (a modified version of SEQ ID No. 13 with foldon and linker sequence); the fourth being 2 doses of the Moderna vaccine followed by a dose of saline; and the final being 3 doses of saline.

FIG. 14 illustrates Separate dosage regimens were compared: the first being 3 doses of the Sinovac vaccine; the second being 2 doses of the Sinovac vaccine followed by a dose of 25 μg of the protein listed in SEQ ID No. 164 (a modified version of SEQ ID No. 1 with foldon and linker sequence) administered with Sigma adjuvant; the third being 2 doses of the Sinovac vaccine followed by a dose of 25 μg of the protein listed in SEQ ID No. 165 (a modified version of SEQ ID No. 13 with foldon and linker sequence) administered with Sigma adjuvant; the fourth being 2 doses of the Sinovac vaccine followed by a dose of 1 μg of the protein listed in SEQ ID No. 165 (a modified version of SEQ ID No. 13 with foldon and linker sequence); and the final being 3 doses of saline.

### DETAILED DESCRIPTION

The present disclosure provides proteins comprising an amino acid sequence from sarbecovirus Spike proteins. Such polypeptides are exemplified below.

Throughout this document, unless otherwise indicated to the contrary, the terms “comprising”, “consisting of”, “having” and the like, are to be construed as non-exhaustive, or in other words, as meaning “including, but not limited to”.

Furthermore, throughout the specification, unless the context requires otherwise, the word “include” or variations such as “includes” or “including” will be understood to imply the inclusion of a stated integer or group of integers but not the exclusion of any other integer or group of integers.

Unless defined otherwise, all technical and scientific terms used herein have the same meaning as is commonly understood by a skilled person to which the subject matter herein belongs.

According to various embodiments there is an amino acid construct comprising any one of the amino acid sequence selected from the group comprising SEQ ID Nos. 10 to 22, 164 and 165, the amino acid construct having at least 90% sequence identity to at least two different sarbecovirus Spike proteins, or a fragment thereof.

Throughout the description, it is to be appreciated that the term ‘Sarbecovirus’ and its plural form include any beta coronavirus that uses angiotensin converting enzyme 2 (ACE2) receptor as entry into cells. In various embodiments, the sarbecovirus comprises any beta coronavirus that uses ACE2 receptor as entry into cells. In various embodiments, the sarbecovirus comprises any beta coronavirus that uses human ACE2 receptor as entry into human cells. In various embodiments, the sarbecovirus comprises any known or new sarbecovirus. In various embodiments, the sarbecovirus is selected from the group comprising or consisting of SARS-CoV, SARS-CoV-2, SARS-CoV-2 B.1.1.7, SARS-CoV-2 B.1.351, SC2r-CoV RaTG13, and SC2r-CoV GX-P5L.

In various embodiments, the amino acid construct comprises a sequence designed to have consensus with two or more different sarbecovirus Spike protein sequences, or fragments thereof. Whereby two or more different sarbecovirus Spike protein sequences, or fragments thereof are aligned, and all identical amino acids are retained, and the first variation is arbitrarily selected from at least one of the sarbecovirus Spike protein sequences, or fragments thereof and the subsequent variation is selected from a different sarbecovirus Spike protein sequences, or fragments thereof. The resulting consensus sequence is therefore, similar to and has consensus with the two or more different from sarbecovirus Spike protein sequences, or fragments thereof from which it is derived but it varies from each of these. In various embodiments the construct may be modified with foldon and linker sequence. The advantage is that when such an amino acid construct is used as an immunogenic composition it results in antibodies able to neutralise several different sarbecovirus infections i.e., the protein can be used as a pan-sarbecovirus vaccine.

In various embodiments, the sarbecovirus Spike protein refers to a wild type spike protein identified or isolated from any of sarbecovirus as listed above and a fragment thereof refers to a wild type sarbecovirus receptor binding domain (RBD) of the spike protein that binds to the ACE2 receptor. In various embodiments, the sarbecovirus Spike protein comprises a SARS-CoV spike protein having an amino acid sequence set forth in SEQ ID NO:1. In various embodiments, the sarbecovirus Spike protein comprises a SARS-CoV2 spike protein having an amino acid sequence set forth in SEQ ID NO:2. In various embodiments, the sarbecovirus Spike protein comprises any one of the proteins having an amino acid sequence set forth in SEQ ID NOs: 23-163. In various embodiments, the fragment of the spike protein comprises a SARS-CoV, RBD having an amino acid sequence set forth in SEQ ID NO:4. In various embodiments, the fragment of the spike protein comprises a SARS-CoV2, RBD having an amino acid sequence set forth in SEQ ID NO:7. In various embodiments, the fragment of the spike protein comprises an amino acid sequence set forth in SEQ ID NO:3 or SEQ ID NO: 5. In various embodiments, the amino acid construct has at least 75%, including one of 75%, 80%, 85%, 90%, 91%, 92%, 93%, 94%, 95%, 96%, 97%, 98%, or even 99.9% sequence identity to at least two different sarbecovirus Spike proteins, or a fragment thereof. In various embodiments, the amino acid construct has at least 75%, including one of 75%, 80%, 85%, 90%, 91%, 92%, 93%, 94%, 95%, 96%, 97%, 98%, or even 99.9% sequence identity to both a wild type of a spike protein or a fragment thereof of a first sarbecovirus and a spike protein or a fragment thereof of a second sarbecovirus. In various embodiments, the first sarbecovirus may comprise SARS-CoV and the second sarbecovirus may comprise SC1 r-CoV. In various embodiments, the first sarbecovirus may comprise SARS-CoV and the second sarbecovirus may comprise SARS-CoV-2 B.1.1.7. In various embodiments, the first sarbecovirus may comprise SARS-CoV and the second sarbecovirus may comprise SARS-CoV-2 B.1.351. In various embodiments, the first sarbecovirus may comprise SARS-CoV-2 and the second sarbecovirus may comprise SC2r-CoV. In various embodiments, the first sarbecovirus may comprise SARS-CoV-2 and the second sarbecovirus may comprise SC2r-CoV. In various embodiments, the first sarbecovirus may be selected from any one of SARS-CoV-2, Brazil SARS-CoV-2 variant P.1 also known as 20J/501Y.V3/B.1.1.248, UK SARS-CoV-2 variant B.1.1.7, South African SARS-CoV-2 variant B.1.351 also known as 20H/501Y.V2, or 501Y.V2 variant, Indian SARS-CoV-2 variant B1.617, SC2r-CoV RaTG13, and SC2r-CoV GX-P5L and the second sarbecovirus may be selected from any one of SARS-CoV, SARS-CoV-2, SARS-CoV-2 B.1.1.7, SARS-CoV-2 B.1.351, SARS-CoV-2 B1.617, SC2r-CoV RaTG13, and SC2r-CoV GX-P5L provided it is different from the first sarbecovirus.

Throughout the description, it is to be appreciated that the term ‘isolated’ include those purified by standard purification methods. It does not require absolute purity and can include protein, peptide, nucleic acid or vaccine molecules that are at least 80%, 85%, 90%, 95%, 98%, or 99% isolated.

In various embodiments, the at least two different sarbecovirus Spike proteins or fragment comprises an amino acid sequence having at least 90%, including one of 90%, 91%, 92%, 93%, 94%, 95%, 96%, 97%, 98%, or even 99.9% sequence identity to the at least two sarbecovirus Spike protein, and an amino acid sequence having at least 90%, including one of 90%, 91%, 92%, 93%, 94%, 95%, 96%, 97%, 98%, or even 99.9% sequence identity to two or more different sarbecovirus Spike proteins or fragment thereof. In various embodiments, the at least two different sarbecovirus Spike proteins or fragment comprise a sarbecovirus Spike protein and a fragment of a different sarbecovirus Spike protein.

In various embodiments, the fragment comprises a receptor binding domain fragment of a sarbecovirus Spike protein.

In various embodiments, the amino acid sequence comprises or consists of any one of sequence selected from SEQ ID NOs: 10 to 22 or 165 or any combination thereof. In various embodiments, the amino acid sequence comprises or consists a construct designed to have at least 75% sequence identity with at least two of any one of sequence selected from SEQ ID NOs: 1 to 9 or 23 to 163.

In various embodiments, the amino acid construct comprises a consensus sequence set forth in SEQ ID NO: 10.

In various embodiments, the amino acid construct comprises a consensus sequence set forth in SEQ ID NO: 11.

In various embodiments, the amino acid construct comprises a consensus sequence set forth in SEQ ID NO: 12.

In various embodiments, the amino acid construct comprises a consensus sequence set forth in SEQ ID NO: 13.

In various embodiments, the amino acid construct comprises a consensus sequence set forth in SEQ ID NO: 14.

In various embodiments, the amino acid construct comprises a consensus sequence set forth in SEQ ID NO: 15.

In various embodiments, the amino acid construct comprises a consensus sequence set forth in SEQ ID NO: 16.

In various embodiments, the amino acid construct comprises a consensus sequence set forth in SEQ ID NO: 17.

In various embodiments, the amino acid construct comprises a consensus sequence set forth in SEQ ID NO: 18.

In various embodiments, the amino acid construct comprises a consensus sequence set forth in SEQ ID NO: 19.

In various embodiments, the amino acid construct comprises a consensus sequence set forth in SEQ ID NO: 20.

In various embodiments, the amino acid construct comprises a consensus sequence set forth in SEQ ID NO: 21.

In various embodiments, the amino acid construct comprises a consensus sequence set forth in SEQ ID NO: 22.

In various embodiments, the amino acid construct comprises a foldon and a linker sequence. In various embodiments, the amino acid construct comprising a foldon and a linker sequence comprises consensus sequence set forth in SEQ ID NO: 164 or SEQ ID NO: 165.

In various embodiments, the amino acid construct comprises an oligomeric polypeptide. In various embodiments, the oligomeric polypeptide comprises a hetero-oligomer.

In various embodiments, the polypeptide is a fusion dimer.

According to various embodiments there is a nucleic acid encoding the amino acid construct as described herein above.

In various embodiments, the nucleic acid comprises a messenger ribonucleic acid (mRNA).

In various embodiments, the nucleic acid is a RNA. The term ‘RNA’ includes a ribonucleic acid (RNA) polynucleotide having an open reading frame encoding a polypeptide of the amino acid construct capable of inducing an immune response against one or more sarbecovirus infections. In various embodiment, it includes at least one ribonucleic acid (RNA) polynucleotide having an open reading frame that encodes amino acid construct that can elicit an immune response against two or more different sarbecovirus infections wherein the amino acid constructs are as described herein above.

According to various embodiments there is an immunogenic composition comprising amino acid construct as described herein above or the nucleic acid as described herein above.

In various embodiments, the immunogenic composition includes a recombinant sarbecovirus spike ectodomain trimer that elicits or induces a measurable response against the sarbecovirus when administered to a subject. For in vivo use, the immunogenic composition will typically include the recombinant coronavirus spike ectodomain trimer or a nucleic acid molecule encoding a protomer of the recombinant coronavirus spike ectodomain trimer in a pharmaceutically acceptable carrier and may also include other agents, such as an adjuvant.

In various embodiments, the adjuvant includes MF59, Adjuvant System 03 (A S03), CpG 1018 or Sigma Adjuvant System (S6322).

In various embodiments, the immunogenic composition comprises at least two amino acid constructs capable of eliciting or inducing a measurable response against the sarbecovirus when administered to a subject. In various embodiments the subject or individual is an animal, such as a mammal such as a human at risk of coming into contact with a sarbecovirus infection.

In various embodiments, the immunogenic composition is an RNA vaccine. The term ‘RNA vaccine’ includes a vaccine having a ribonucleic acid (RNA) polynucleotide having an open reading frame encoding the amino acid construct capable of inducing an immune response against sarbecovirus infections. In various embodiments, the immunogenic composition includes at least one ribonucleic acid (RNA) polynucleotide having an open reading frame that encodes two or more the amino acid constructs that can elicit an immune response against sarbecovirus infections.

In various embodiments, the immunogenic composition comprises the amino acid construct as described herein above, or the nucleic acid as described herein above enclosed in a lipid nanoparticles. In various embodiments, the immunogenic composition is a mRNA vaccine comprising lipid nanoparticles. In various embodiments, the immunogenic composition comprises a pre-fusion stabilized spike-RBD-adjuvant trimer.

In various embodiments, the amino acid construct having at least 75% sequence identity to sarbecovirus RBD fragment comprises a recombinant RBD (rRBD). In various embodiments, the immunogenic composition comprises two dimeric formats selected from tandem dimer and rRBD-Fc fusion dimer.

In various embodiments, the immunogenic composition comprises a carrier. In various embodiments, the carrier is any one of a lipid nanoparticle (LNP), a polymeric nanoparticle, a lipid carrier such as a lipidoid, a liposome, a lipoplex, a peptide carrier, a nanoparticle mimic, a nanotube, or a conjugate.

According to various embodiments there is a viral vector comprising a nucleic acid as described herein above.

In various embodiments, the vector is selected from a recombinant measles virus vector, a vesicular stomatitis virus (VSV) vector, a vaccinia virus vector or an adenovirus vector.

According to various embodiments there is an amino acid construct as described herein above for use in the treatment or prevention of sarbecovirus infections.

According to various embodiments there is an immunogenic composition as described herein above for use in the treatment and/or prevention of sarbecovirus infections.

According to various embodiments there is use of an amino acid construct as described herein above in the manufacture of a medicament for the treatment and/or prevention of sarbecovirus infections.

According to various embodiments there is use of an immunogenic composition as described herein above in the manufacture of a medicament for the treatment and/or prevention of sarbecovirus infections.

According to various embodiments there is use of a method of treating and/or preventing an infection caused by sarbecovirus comprising administering a vaccine molecule comprising an amino acid construct as described herein above, an immunogenic composition as described herein above, or a viral vector as described herein above to a subject.

In various embodiments, a method for inducing an immune response to a mammalian subject comprises administering a therapeutically effective amount of the immunogenic composition as described herein above, to the mammalian subject.

According to various embodiments, there is a method of making an amino acid construct as described herein above, the method comprising: a) comparing amino acid sequences from at least two different sarbecovirus spike proteins or fragments thereof; b) identifying identical amino acid in the sequences from the at least two different sarbecovirus spike proteins or fragments thereof; c) removing any different amino acids from the sequences of the at least two different sarbecovirus spike proteins or fragments thereof to identify a unique amino acid sequence; and d) forming the amino construct of unique amino acid sequence wherein the amino construct has at least 75% sequence identity to the at least two different sarbecovirus Spike protein, or a fragment thereof.

In various embodiments, the method of making an amino acid construct further comprising modifying the unique amino acid sequence with a foldon and a linker sequence. The C-terminal domain of T4 fibritin (foldon) is known for the formation of the fibritin trimer structure and can be used as an artificial trimerization domain. In various embodiments the linker comprises a His6-tag.

In various embodiments, the at least two different sarbecovirus spike proteins or fragments thereof comprise any one of 2, 5, 10, 20, 30, 40, 50, 60, 70, 80, 90, 100, 110, 120, 130, 140, 150, 160, and 170 different sarbecovirus spike proteins or fragments thereof. In various embodiments the at least two different sarbecovirus spike proteins or fragments thereof comprise a plurality of sarbecovirus spike proteins or fragments thereof. In various embodiments, the plurality of sarbecovirus spike proteins or fragments thereof comprise 50-100, 80-150, 100-200 different sarbecovirus spike proteins or fragments thereof. In various embodiments step a) comprises comparing amino acid sequences from a plurality of different sarbecovirus spike proteins or fragments thereof, and step b) comprises identifying amino acid sequences from the plurality of different sarbecovirus spike proteins or fragments thereof above a predetermined sequence identity to the majority of the plurality of different sarbecovirus spike proteins or fragments thereof and step c) comprises removing any amino acids sequences from the plurality of different sarbecovirus spike proteins or fragments thereof below a predetermined sequence identity to the majority of the plurality of different sarbecovirus spike proteins or fragments thereof. In various embodiments the predetermined sequence identity is 85% or 90% or 95%, or 96% or 97%, or 98% or 99%.

In various embodiments, the method further comprising identifying a fragment within the unique amino acid sequence that corresponds to a receptor binding domain of the at least two different sarbecovirus spike proteins to form a unique amino acid fragment sequence; and forming the amino construct of the combined the unique amino acid fragment sequence with the unique amino acid sequence.

In various embodiments, forming the amino construct in step d) comprises forming a nucleic acid encoding the amino acid construct, able to express the amino acid construct.

In various embodiments, the nucleic acid comprises a messenger ribonucleic acid (mRNA).

In various embodiments, the amino acid construct or a nucleic acid encoding the amino acid construct able to express the amino acid construct is prepared as an immunogenic composition.

In various embodiments, the immunogenic composition comprises an adjuvant.

In various embodiments, the nucleic acid is prepared in a viral vector.

In various embodiments, the vector is selected from a recombinant measles virus vector, a vesicular stomatitis virus (VSV) vector, a vaccinia virus vector or an adenovirus vector.

Throughout the description, it is to be appreciated that the term ‘polypeptide’, used interchangeably with protein or peptide, includes amino acid polymers including naturally occurring amino acid polymers and non-naturally occurring amino acid polymers. A polypeptide has an amino terminal (N-terminal) end and a carboxy terminal (C-terminal) end.

In various embodiments the amino acid construct comprises a consensus amino acid sequence as set forth in SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17, SEQ ID NO: 18; SEQ ID NO: 19; SEQ ID NO: 20; SEQ ID NO: 21; SEQ ID NO: 22 SEQ ID NO: 164 or SEQ ID NO: 165.

In various embodiments, the amino acid construct having at least 75% sequence identity to a sarbecovirus spike protein, comprises amino acid sequence as set forth in SEQ ID NO:1; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 164; SEQ ID NO: 165.

In various embodiments, there is the amino acid construct having at least 75% sequence identity to a sarbecovirus receptor binding domain (RBD) protein comprises a consensus amino acid sequence as set forth in SEQ ID NO: 4; SEQ ID NO: 18; SEQ ID NO: 19; SEQ ID NO: 20; SEQ ID NO: 21; SEQ ID NO: 22; SEQ ID NO: 164; or SEQ ID NO: 165.

In various embodiments, the amino acid construct having at least 75% sequence identity to a sarbecovirus spike protein may have a minimum length of one of 25, 50, 100, 200, 300, 400, 500, 600, 700, 800, 900, 1,000, 1,100 or 1,200 amino acids, and may have a maximum length of one of 50, 100, 200, 300, 400, 500, 600, 700, 800, 900, 1,000, 1,100 or 1,200 amino acids.

In various embodiments, the amino acid construct having at least 75% sequence identity to a sarbecovirus receptor binding domain (RBD) protein may have a minimum length of one of 10, 20, 30, 40, 50, 100, 150, 200 amino acids, and may have a maximum length of one of 20, 10, 20, 30, 40, 50, 100, 150, 200 amino acids.

### EXAMPLES

### Sequences

| SEQ | | |
|---|---|---|
| ID | | |
| NO: | DESCRIPTION | AMINO ACID SEQUENCE |
| 1 | SARS-CoV | MFIFLLFLTLTSGSDLDRCTTFDDVQAPNYTQHTSSMRGVYYP |
| | spike protein | DEIFRSDTLYLTQDLFLPFYSNVTGFHTINHTFDNPVIPFKDGIY |
| | | FAATEKSNVVRGWVFGSTMNNKSQSVIIINNSTNVVIRACNFE |
| | | LCDNPFFAVSKPMGTQTHTMIFDNAFNCTFEYISDAFSLDVSE |
| | | KSGNFKHLREFVFKNKDGFLYVYKGYQPIDVVRDLPSGFNTLK |
| | | PIFKLPLGINITNFRAILTAFSPAQDTWGTSAAAYFVGYLKPTTF |
| | | MLKYDENGTITDAVDCSQNPLAELKCSVKSFEIDKGIYQTSNF |
| | | RVVPSGDVVRFPNITNLCPFGEVFNATKFPSVYAWERKKISNC |
| | | VADYSVLYNSTFFSTFKCYGVSATKLNDLCFSNVYADSFVVKG |
| | | DDVRQIAPGQTGVIADYNYKLPDDFMGCVLAWNTRNIDATST |
| | | GNYNYKYRYLRHGKLRPFERDISNVPFSPDGKPCTPPALNCY |
| | | WPLNDYGFYTTTGIGYQPYRVVVLSFELLNAPATVCGPKLSTD |
| | | LIKNQCVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAEHVDTSYE |
| | | CDIPIGAGICASYHTVSLLRSTSQKSIVAYTMSLGADSSIAYSNN |
| | | TIAIPTNFSISITTEVMPVSMAKTSVDCNMYICGDSTECANLLLQ |
| | | YGSFCTQLNRALSGIAAEQDRNTREVFAQVKQMYKTPTLKYF |
| | | GGFNFSQILPDPLKPTKRSFIEDLLFNKVTLADAGFMKQYGEC |
| | | LGDINARDLICAQKFNGLTVLPPLLTDDMIAAYTAALVSGTATA |
| | | GWTFGAGAALQIPFAMQMAYRFNGIGVTQNVLYENQKQIANQ |
| | | FNKAISQIQESLTTTSTALGKLQDVVNQNAQALNTLVKQLSSNF |
| | | GAISSVLNDILSRLDKVEAEVQIDRLITGRLQSLQTYVTQQLIRA |
| | | AEIRASANLAATKMSECVLGQSKRVDFCGKGYHLMSFPQAAP |
| | | HGVVFLHVTYVPSQERNFTTAPAICHEGKAYFPREGVFVFNG |
| | | TSWFITQRNFFSPQIITTDNTFVSGNCDVVIGIINNTVYDPLQPE |
| | | LDSFKEELDKYFKNHTSPDVDLGDISGINASVVNIQKEIDRLNE |
| | | VAKNLNESLIDLQELGKYEQYIKWPWYVWLGFIAGLIAIVMVTIL |
| | | LCCMTSCCSCLKGACSCGSCCKFDEDDSEPVLKGVKLHYT |
| 2 | SARS-CoV-2 | MFLLTTKRTMFVFLVLLPLVSSQCVNLTTRTQLPPAYTNSFTR |
| | spike protein | GVYYPDKVFRSSVLHSTQDLFLPFFSNVTWFHAIHVSGTNGTK |
| | | RFDNPVLPFNDGVYFASTEKSNIIRGWIFGTTLDSKTQSLLIVN |
| | | NATNVVIKVCEFQFCNDPFLGVYYHKNNKSWMESEFRVYSSA |
| | | NNCTFEYVSQPFLMDLEGKQGNFKNLREFVFKNIDGYFKIYSK |
| | | HTPINLVRDLPQGFSALEPLVDLPIGINITRFQTLLALHRSYLTP |
| | | GDSSSGWTAGAAAYYVGYLQPRTFLLKYNENGTITDAVDCAL |
| | | DPLSETKCTLKSFTVEKGIYQTSNFRVQPTESIVRFPNITNLCP |
| | | FGEVFNATRFASVYAWNRKRISNCVADYSVLYNSASFSTFKC |
| | | YGVSPTKLNDLCFTNVYADSFVIRGDEVRQIAPGQTGKIADYN |
| | | YKLPDDFTGCVIAWNSNNLDSKVGGNYNYLYRLFRKSNLKPF |
| | | ERDISTEIYQAGSTPCNGVEGFNCYFPLQSYGFQPTNGVGYQ |
| | | PYRVVVLSFELLHAPATVCGPKKSTNLVKNKCVNFNFNGLTGT |
| | | GVLTESNKKFLPFQQFGRDIADTTDAVRDPQTLEILDITPCSFG |
| | | GVSVITPGTNTSNQVAVLYQDVNCTEVPVAIHADQLTPTWRVY |
| | | STGSNVFQTRAGCLIGAEHVNNSYECDIPIGAGICASYQTQTN |
| | | SPRRARSVASQSIIAYTMSLGAENSVAYSNNSIAIPTNFTISVTT |
| | | EILPVSMTKTSVDCTMYICGDSTECSNLLLQYGSFCTQLNRAL |
| | | TGIAVEQDKNTQEVFAQVKQIYKTPPIKDFGGFNFSQILPDPSK |
| | | PSKRSFIEDLLFNKVTLADAGFIKQYGDCLGDIAARDLICAQKF |
| | | NGLTVLPPLLTDEMIAQYTSALLAGTITSGWTFGAGAALQIPFA |
| | | MQMAYRFNGIGVTQNVLYENQKLIANQFNSAIGKIQDSLSSTA |
| | | SALGKLQDVVNQNAQALNTLVKQLSSNFGAISSVLNDILSRLD |
| | | KVEAEVQIDRLITGRLQSLQTYVTQQLIRAAEIRASANLAATKM |
| | | SECVLGQSKRVDFCGKGYHLMSFPQSAPHGVVFLHVTYVPA |
| | | QEKNFTTAPAICHDGKAHFPREGVFVSNGTHWFVTQRNFYEP |
| | | QIITTDNTFVSGNCDVVIGIVNNTVYDPLQPELDSFKEELDKYF |
| | | KNHTSPDVDLGDISGINASVVNIQKEIDRLNEVAKNLNESLIDLQ |
| | | ELGKYEQYIKWPWYIWLGFIAGLIAIVMVTIMLCCMTSCCSCLK |
| | | GCCSCGSCCKFDEDDSEPVLKGVKLHYT |
| 3 | SARS-CoV | SDLDRCTTFDDVQAPNYTQHTSSMRGVYYPDEIFRSDTLYLT |
| | spike protein | QDLFLPFYSNVTGFHTINHTFDNPVIPFKDGIYFAATEKSNVVR |
| | S1 subunit | GWVFGSTMNNKSQSVIIINNSTNVVIRACNFELCDNPFFAVSK |
| | | PMGTQTHTMIFDNAFNCTFEYISDAFSLDVSEKSGNFKHLREF |
| | | VFKNKDGFLYVYKGYQPIDVVRDLPSGFNTLKPIFKLPLGINITN |
| | | FRAILTAFSPAQDTWGTSAAAYFVGYLKPTTFMLKYDENGTIT |
| | | DAVDCSQNPLAELKCSVKSFEIDKGIYQTSNFRVVPSGDVVRF |
| | | PNITNLCPFGEVFNATKFPSVYAWERKKISNCVADYSVLYNST |
| | | FFSTFKCYGVSATKLNDLCFSNVYADSFVVKGDDVRQIAPGQ |
| | | TGVIADYNYKLPDDFMGCVLAWNTRNIDATSTGNYNYKYRYL |
| | | RHGKLRPFERDISNVPFSPDGKPCTPPALNCYWPLNDYGFYT |
| | | TTGIGYQPYRVVVLSFELLNAPATVCGPKLSTDLIKNQCVNFNF |
| | | NGLTGTGVLTPSSKRFQPFQQFGRDVSDFTDSVRDPKTSEIL |
| | | DISPCSFGGVSVITPGTNASSEVAVLYQDVNCTDVSTAIHADQ |
| | | LTPAWRIYSTGNNVFQTQAGCLIGAEHVDTSYECDIPIGAGICA |
| | | SYHTVSLLR |
| 4 | SARS-CoV | RVVPSGDVVRFPNITNLCPFGEVFNATKFPSVYAWERKKISNC |
| | spike protein | VADYSVLYNSTFFSTFKCYGVSATKLNDLCFSNVYADSFVVKG |
| | RBD | DDVRQIAPGQTGVIADYNYKLPDDFMGCVLAWNTRNIDATST |
| | | GNYNYKYRYLRHGKLRPFERDISNVPFSPDGKPCTPPALNCY |
| | | WPLNDYGFYTTTGIGYQPYRVVVLSFELLNAPATVCGPKLSTD |
| | | LIKNQCVNF |
| 5 | SARS-CoV-2 | SQCVNLTTRTQLPPAYTNSFTRGVYYPDKVFRSSVLHSTQDL |
| | spike protein | FLPFFSNVTWFHAIHVSGTNGTKRFDNPVLPFNDGVYFASTEK |
| | S1 subunit | SNIIRGWIFGTTLDSKTQSLLIVNNATNVVIKVCEFQFCNDPFLG |
| | | VYYHKNNKSWMESEFRVYSSANNCTFEYVSQPFLMDLEGKQ |
| | | GNFKNLREFVFKNIDGYFKIYSKHTPINLVRDLPQGFSALEPLV |
| | | DLPIGINITRFQTLLALHRSYLTPGDSSSGWTAGAAAYYVGYLQ |
| | | PRTFLLKYNENGTITDAVDCALDPLSETKCTLKSFTVEKGIYQT |
| | | SNFRVQPTESIVRFPNITNLCPFGEVFNATRFASVYAWNRKRI |
| | | SNCVADYSVLYNSASFSTFKCYGVSPTKLNDLCFTNVYADSFV |
| | | IRGDEVRQIAPGQTGKIADYNYKLPDDFTGCVIAWNSNNLDSK |
| | | VGGNYNYLYRLFRKSNLKPFERDISTEIYQAGSTPCNGVEGEN |
| | | CYFPLQSYGFQPTNGVGYQPYRVVVLSFELLHAPATVCGPKK |
| | | STNLVKNKCVNFNFNGLTGTGVLTESNKKFLPFQQFGRDIADT |
| | | TDAVRDPQTLEILDITPCSFGGVSVITPGTNTSNQVAVLYQDVN |
| | | CTEVPVAIHADQLTPTWRVYSTGSNVFQTRAGCLIGAEHVNN |
| | | SYECDIPIGAGICASYQTQTNSPRRAR |
| 6 | SARS-CoV-2 | RVQPTESIVRFPNITNLCPFGEVFNATRFASVYAWNRKRISNC |
| | spike protein | VADYSVLYNSASFSTFKCYGVSPTKLNDLCFTNVYADSFVIRG |
| | RBD | DEVRQIAPGQTGKIADYNYKLPDDFTGCVIAWNSNNLDSKVG |
| | | GNYNYLYRLFRKSNLKPFERDISTEIYQAGSTPCNGVEGENCY |
| | | FPLQSYGFQPTNGVGYQPYRVVVLSFELLHAPATVCGPKKST |
| | | NLVKNKCVNF |
| 7 | SARS-CoV-2 | PNITNLCPFGEVFNATRFASVYAWNRKRISNCVADYSVLYNSA |
| | spike protein | SFSTFKCYGVSPTKLNDLCFTNVYDSFVIRGDEVRQIAPGQTG |
| | RBD | KIADYNYKLPDDFTGCVIAWNSNNLDSKVGGNYNYLYRLFRKS |
| | | NLKPFERDISTEIYQAGSTPCNGVEGFNCYFPLQSYGFQPTNG |
| | | VGYQPYRVVVLSFELLHAPATVCGPKKS |
| 8 | SARS-CoV-2 | MLLVNQSHQGFNKEHTSKMVSAIVLYVLLAAAAHSAFAQCVNL |
| | spike protein | TTRTQLPPAYTNSFTRGVYYPDKVFRSSVLHSTQDLFLPFFSN |
| | S1 subunit | VTWFHAIHVSGTNGTKRFDNPVLPFNDGVYFASTEKSNIIRGW |
| | | IFGTTLDSKTQSLLIVNNATNVVIKVCEFQFCNDPFLGVYYHKN |
| | | NKSWMESEFRVYSSANNCTFEYVSQPFLMDLEGKQGNFKNL |
| | | REFVFKNIDGYFKIYSKHTPINLVRDLPQGFSALEPLVDLPIGINI |
| | | TRFQTLLALHRSYLTPGDSSSGWTAGAAAYYVGYLQPRTFLL |
| | | KYNENGTITDAVDCALDPLSETKCTLKSFTVEKGIYQTSNFRV |
| | | QPTESIVRFPNITNLCPFGEVFNATRFASVYAWNRKRISNCVA |
| | | DYSVLYNSASFSTFKCYGVSPTKLNDLCFTNVYADSFVIRGDE |
| | | VRQIAPGQTGKIADYNYKLPDDFTGCVIAWNSNNLDSKVGGN |
| | | YNYLYRLFRKSNLKPFERDISTEIYQAGSTPCNGVEGFNCYFP |
| | | LQSYGFQPTNGVGYQPYRVVVLSFELLHAPATVCGPKKSTNL |
| | | VKNKCVNFNFNGLTGTGVLTESNKKFLPFQQFGRDIADTTDAV |
| | | RDPQTLEILDITPCSFGGVSVITPGTNTSNQVAVLYQDVNCTEV |
| | | PVAIHADQLTPTWRVYSTGSNVFQTRAGCLIGAEHVNNSYEC |
| | | DIPIGAGICASYQTQTNSPRRAR |
| 9 | SARS-CoV | MNITNLCPFGEVFNATKFPSVYAWERKKISNCVADYSVLYNST |
| | spike protein | FFSTFKCYGVSATKLNDLCFSNVYADSFVVKGDDVRQIAPGQ |
| | RBD | TGVIADYNYKLPDDFMGCVLAWNTRNIDATSTGNYNYKYRYL |
| | | RHGKLRPFERDISNVPFSPDGKPCTPPALNCYWPLNDYGFYT |
| | | TTGIGYQPYRVVVLSFELLNAPATYLSLNTAAAL |
| 10 | Artificial | MFIFLFFLTLVSGSTIEKCTTFDDVQPPNYTQFLSSHRGVYYPD |
| | Sarbecovirus | DIFRSDVLHLTQDHFLPFDSNVTGFITFGLNFDNPIIPFKDGIYF |
| | spike | AATEKSNVIRGWVFGSTMNNKSQSVIIMNNSTNVVIRACNFEL |
| | consensus 1 | CDNPFFAVSKPNNTQIPSYIFNNAFNCTFEYVSDDFNLDVGEK |
| | | PGNFKHLREFVFKNKDGFLYVYSGYQPIDVASGLPSGFNALKP |
| | | IFKLPLGINITNFRTLLTAFPPRPDYWGTSAAAYFVGYLKPTTF |
| | | MLKYDENGTITDAVDCSQNPLAELKCSVKSFEIDKGIYQTSNF |
| | | RVVPSGDVVRFPNITNLCPFGEVFNATKFPSVYAWERKRISNC |
| | | VADYSVLYNSTFFSTFKCYGVSATKLNDLCFSNVYADSFVVKG |
| | | DDVRQIAPGQTGVIADYNYKLPDDFMGCVLAWNTRNIDATST |
| | | GNYNYKYRYLRHGKLRPFERDISNVPFSPDGKPCTPPALNCY |
| | | WPLNDYGFYTTTGIGYQPYRVVVLSFELLNAPATVCGPKLSTD |
| | | LIKNQCVNFNFNGLTGTGVLTPSSKRFQPFQQFGRDVSDFTD |
| | | SVRDPKTSEILDISPCSFGGVSVITPGTNTSSEVAVLYQDVNCT |
| | | DVPVAIHADQLTPAWRVYSTGNNVFQTQAGCLIGAEHVDTSY |
| | | ECDIPIGAGICASYHTVSSLRSTSQKSIVAYTMSLGADSSIAYS |
| | | NNTIAIPTNFSISITTEVMPVSMAKTSVDCNMYICGDSTECANLL |
| | | LQYGSFCTQLNRALSGIAVEQDRNTREVFAQVKQMYKTPTLK |
| | | DFGGFNFSQILPDPLKPTKRSFIEDLLFNKVTLADAGFMKQYG |
| | | ECLGDINARDLICAQKFNGLTVLPPLLTDDMIAAYTAALVSGTA |
| | | TAGWTFGAGAALQIPFAMQMAYRFNGIGVTQNVLYENQKQIA |
| | | NQFNKAISQIQESLTTTSTALGKLQDVVNQNAQALNTLVKQLS |
| | | SNFGAISSVLNDILSRLDPPEAEVQIDRLITGRLQSLQTYVTQQ |
| | | LIRAAEIRASANLAATKMSECVLGQSKRVDFCGKGYHLMSFPQ |
| | | AAPHGVVFLHVTYVPSQERNFTTAPAICHEGKAYFPREGVFVF |
| | | NGTSWFITQRNFFSPQIITTDNTFVSGSCDVVIGIINNTVYDPLQ |
| | | PELDSFKEELDKYFKNHTSPDVDLGDISGINASVVNIQKEIDRL |
| | | NEVAKNLNESLIDLQELGKYEQYIKWPWYVWLGFIAGLIAIVMV |
| | | TILLCCMTSCCSCLKGACSCGSCCKFDEDDSEPVLKGVKLHY |
| | | T |
| 11 | Artificial | MFIFLLFFTLTSGSDLDRCTTFDDVQAPNYTQHTSSMRGVYYP |
| | Sarbecovirus | DEIFRSDTLYLTQDLFLPFYSNVTGFHTINHTFDNPVIPFKDGIY |
| | spike | FAATEKSNVVRGWVFGSTMNNKSQSVIIINNSTNVVIRACNFE |
| | consensus 2 | LCDNPFFVVSKPMGTRTHTMIFDNAFNCTFEYISDAFSLDVSE |
| | | KSGNFKHLREFVFKNKDGFLYVYKGYQPIDVVRDLPSGFNTLK |
| | | PIFKLPLGIKITNFRAILTAFSPAQGTWGTSAAAYFVGYLKPTTF |
| | | MLKYDENGTITDAVDCSQNPLAELKCSVKSFEIDKGIYQTSNF |
| | | RVVPSGDVVRFPNITNLCPFGEVFNATKFPSVYAWERKRISNC |
| | | VADYSVLYNSTSFSTFKCYGVSATKLNDLCFSNVYADSFVVKG |
| | | DDVRQIAPGQTGVIADYNYKLPDDFMGCVLAWNTRNIDATST |
| | | GNHNYKYRYLRHGKLRPFERDISNVPFSPDGKPCTPPAPNCY |
| | | WPLRGYGFYTTSGIGYQPYRVVVLSFELLNAPATVCGPKLSTD |
| | | LIKNQCVNFNFNGLTGTGVLTPSSKRFQPFQQFGRDVSDFTD |
| | | SVRDPKTSEILDISPRSFGGVSVITPGTNASSEVAVLYQDVNCT |
| | | DVSTLIHAEQLTPAWRIYSTGNNVFQTQAGCLIGAEHVDTSYE |
| | | CDIPIGAGICASYHTVSSLRSTSQKSIVAYTMSLGADSSIAYSN |
| | | NTIAIPTNFSISITTEVMPVSMTKTSVDCNMYICGDSTECANLLL |
| | | QYGSFCRQLNRALSGIAAEQDRNTREVFVQVKQMYKTPTLKD |
| | | FGGFNFSQILPDPLKPTKRSFIEDLLFNKVTLADAGFMKQYGE |
| | | CLGDINARDLICAQKFNGLTVLPPLLTVDMIAAYTAALVSGTAT |
| | | AGWTFGAGAALQIPFAMQMAYRFNGIGVTQNVLYENQKQIAN |
| | | QFNKAISQIQESLTTTSTALGKLQDVVNQNAQALNTLVKQLSS |
| | | NFGAISSVLNDILSRLDPPEAEVQIDRLITGRLQSLQTYVTQQLI |
| | | RAAEIRASANLAATKMSECVLGQSKRVGFCGKGYHLMSFPQA |
| | | APHGVVFLHVTYVPSQERNFTTAPAICHEGKAYFPREGVFVFS |
| | | GTSWFITQRNFFSPQIITTDNTFVSGNCDVVIGIINNTVYDPLQP |
| | | ELDSFKEELDKYFKNHTSPDVDLGDISGINASVVNIQEEIDRLN |
| | | EVAKNLNESLIDLQELGKYEQYIKWPWYVWLGFIAGLIAIVMVTI |
| | | LLCCMTSCCSCLKGACSCGSCCKFDEDDSEPVLKGVKLHYT |
| 12 | Artificial | MFIFLFFLTLVSGSDLESCTTFDDVQAPNYPQHSSSRRGVYYP |
| | Sarbecovirus | DEIFRSDTLYLTQDLFLPFDSNVTGFHTINHRFDNPVIPFKDGV |
| | spike | YFAATEKSNVVRGWVFGSTMNNKSQSVIIINNSTNVVIRACNF |
| | consensus 3 | ELCDNPFFAVSKPTGTQTHTMIFNNAFNCTFEYISDSFSLDVA |
| | | EKSGNFKHLREFVFKNKDGFLYVYKGYQPIDVVRDLPSGFNIL |
| | | KPIFKLPLGINITNFRAILTAFLPAQDTWGTSAAAYFVGYLKPAT |
| | | FMLKYDENGTITDAVDCSQNPLAELKCSVKSFEIDKGIYQTSNF |
| | | RVAPSKEVVRFPNITNLCPFGEVFNATTFPSVYAWERKRISNC |
| | | VADYSVLYNSTSFSTFKCYGVSATKLNDLCFSNVYADSFVVKG |
| | | DDVRQIAPGQTGVIADYNYKLPDDFTGCVLAWNTRNIDATQT |
| | | GNYNYKYRSLRHGKLRPFERDISNVPFSPDGKPCTPPAFNCY |
| | | WPLNDYGFYTTNGIGYQPYRVVVLSFELLNAPATVCGPKLSTD |
| | | LIKNQCVNFNFNGLTGTGVLTPSSKRFQPFQQFGRDVSDFTD |
| | | SVRDPKTSEILDISPCSFGGVSVITPGTNTSSEVAVLYQDVNCT |
| | | DVPVAIHADQLTPAWRIYSTGNNVFQTQAGCLIGAEHVDTSYE |
| | | CDIPIGAGICASYHTVSSLRSTSQKSIVAYTMSLGADSSIAYSN |
| | | NTIAIPTNFSISITTEVMPVSMAKTSVDCNMYICGDSTECANLLL |
| | | QYGSFCTQLNRALSGIAVEQDRNTREVFAQVKQMYKTPTLKD |
| | | FGGFNFSQILPDPLKPTKRSFIEDLLFNKVTLADAGFMKQYGE |
| | | CLGDINARDLICAQKFNGLTVLPPLLTDDMIAAYTAALVSGTAT |
| | | AGWTFGAGAALQIPFAMQMAYRFNGIGVTQNVLYENQKQIAN |
| | | QFNKAISQIQESLTTTSTALGKLQDVVNQNAQALNTLVKQLSS |
| | | NFGAISSVLNDILSRLDPPEAEVQIDRLITGRLQSLQTYVTQQLI |
| | | RAAEIRASANLAATKMSECVLGQSKRVDFCGKGYHLMSFPQA |
| | | APHGVVFLHVTYVPSQERNFTTAPAICHEGKAYFPREGVFVFN |
| | | GTSWFITQRNFFSPQIITTDNTFVSGNCDVVIGIINNTVYDPLQP |
| | | ELDSFKEELDKYFKNHTSPDVDLGDISGINASVVNIQKEIDRLN |
| | | EVAKNLNESLIDLQELGKYEQYIKWPWYVWLGFIAGLIAIVMVTI |
| | | LLCCMTSCCSCLKGACSCGSCCKFDEDDSEPVLKGVKLHYT |
| 13 | Artificial | MFIFLFFLTLVSGSTIEKCTTFDDVQPPNYTQFLSSHRGVYYPD |
| | Sarbecovirus | DIFRSDVLHLTQDHFLPFDSNVTGFITFGLNFDNPIIPFKDGIYF |
| | spike | AATEKSNVIRGWVFGSTMNNKSQSVIIMNNSTNVVIRACNFEL |
| | consensus 4 | CDNPFFAVSKPNNTQIPSYIFNNAFNCTFEYVSDDFNLDVGEK |
| | | PGNFKHLREFVFKNKDGFLYVYSGYQPIDVASGLPSGFNALKP |
| | | IFKLPLGINITNFRTLLTAFPPRPDYWGTSAAAYFVGYLKPTTF |
| | | MLKYDENGTITDAVDCSQNPLAELKCSVKSFEIDKGIYQTSNF |
| | | RVAPSKEVVRFPNITNLCPFGEVFNATTFPSVYAWERKRISNC |
| | | VADYSVLYNSTSFSTFKCYGVSATKLNDLCFSNVYADSFVVKG |
| | | DDVRQIAPGQTGVIADYNYKLPDDFMGCVLAWNTRNIDATST |
| | | GNYNYKYRSLRHGKLRPFERDISNVPFSPDGKPCTPPAFNCY |
| | | WPLNDYGFFTTNGIGYQPYRVVVLSFELLNAPATVCGPKLSTD |
| | | LIKNQCVNFNFNGLTGTGVLTPSSKRFQPFQQFGRDVSDFTD |
| | | SVRDPKTSEILDISPCSFGGVSVITPGTNTSSEVAVLYQDVNCT |
| | | DVPVAIHADQLTPAWRVYSTGNNVFQTQAGCLIGAEHVDTSY |
| | | ECDIPIGAGICASYHTVSSLRSTSQKSIVAYTMSLGADSSIAYS |
| | | NNTIAIPTNFSISITTEVMPVSMAKTSVDCNMYICGDSTECANLL |
| | | LQYGSFCTQLNRALSGIAVEQDRNTREVFAQVKQMYKTPTLK |
| | | DFGGFNFSQILPDPLKPTKRSFIEDLLFNKVTLADAGFMKQYG |
| | | ECLGDINARDLICAQKFNGLTVLPPLLTDDMIAAYTAALVSGTA |
| | | TAGWTFGAGAALQIPFAMQMAYRFNGIGVTQNVLYENQKQIA |
| | | NQFNKAISQIQESLTTTSTALGKLQDVVNQNAQALNTLVKQLS |
| | | SNFGAISSVLNDILSRLDPPEAEVQIDRLITGRLQSLQTYVTQQ |
| | | LIRAAEIRASANLAATKMSECVLGQSKRVDFCGKGYHLMSFPQ |
| | | AAPHGVVFLHVTYVPSQERNFTTAPAICHEGKAYFPREGVFVF |
| | | NGTSWFITQRNFFSPQIITTDNTFVSGSCDVVIGIINNTVYDPLQ |
| | | PELDSFKEELDKYFKNHTSPDVDLGDISGINASVVNIQKEIDRL |
| | | NEVAKNLNESLIDLQELGKYEQYIKWPWYVWLGFIAGLIAIVMV |
| | | TILLCCMTSCCSCLKGACSCGSCCKFDEDDSEPVLKGVKLHY |
| | | T |
| 14 | Artificial | MFVFLFLLPLVSSQCVNLTTRTGIPPGYTNSSTRGVYYPDKVF |
| | Sarbecovirus | RSSVLHLTQDLFLPFFSNVTWFNAINYQGGFKRFDNPVLPFND |
| | spike | GVYFASTEKSNIIRGWIFGTTLDARTQSLLIVNNATNVVIKVCEF |
| | consensus 5 | QFCTDPFLGVYYHNNNKTWVENEFRVYSSANNCTFEYVSQP |
| | | FLMDLEGKQGNFKNLREFVFKNVDGYFKIYSKHTPIDLVRDLP |
| | | RGFSALEPLVDLPIGINITRFQTLLALHRSYLTPGKLESGWTTG |
| | | AAAYYVGYLQPRTFLLSYNENGTITDAVDCALDPLSETKCTLK |
| | | SLTVEKGIYQTSNFRVQPTISIVRFPNITNLCPFGEVFNATRFAS |
| | | VYAWNRKRISNCVADYSVLYNSTSFSTFKCYGVSPTKLNDLCF |
| | | TNVYADSFVVRGDEVRQIAPGQTGVIADYNYKLPDDFTGCVIA |
| | | WNSVKQDALTGGNYNYLYRLFRKSKLKPFERDISTEIYQAGST |
| | | PCNGVVGLNCYYPLQRYGFHPTTGVGYQPYRVVVLSFELLNA |
| | | PATVCGPKKSTNLVKNKCVNFNFNGLTGTGVLTESNKKFLPF |
| | | QQFGRDIADTTDAVRDPQTLEILDITPCSFGGVSVITPGTNTSN |
| | | QVAVLYQDVNCTEVPVAIHADQLTPTWRVYSTGSNVFQTRAG |
| | | CLIGAEHVNNSYECDIPIGAGICASYQTQTNSPRRARSVNSQSI |
| | | IAYTMSLGAENSVAYSNNSIAIPTNFTISVTTEILPVSMTKTSVD |
| | | CTMYICGDSIECSNLLLQYGSFCTQLNRALTGIAVEQDKNTQE |
| | | VFAQVKQIYKTPPIKDFGGFNFSQILPDPSKPSKRSFIEDLLFNK |
| | | VTLADAGFIKQYGDCLGDIAARDLICAQKFNGLTVLPPLLTDEM |
| | | IAQYTSALLAGTITSGWTFGAGAALQIPFAMQMAYRFNGIGVT |
| | | QNVLYENQKLIANQFNSAIGKIQDSLSSTASALGKLQDVVNQN |
| | | AQALNTLVKQLSSNFGAISSVLNDILSRLDPPEAEVQIDRLITGR |
| | | LQSLQTYVTQQLIRAAEIRASANLAATKMSECVLGQSKRVDFC |
| | | GKGYHLMSFPQSAPHGVVFLHVTYVPAQEKNFTTAPAICHEG |
| | | KAHFPREGVFVSNGTHWFVTQRNFYEPQIITTDNTFVSGSCD |
| | | VVIGIVNNTVYDPLQPELDSFKEELDKYFKNHTSPDVDLGDISG |
| | | INASVVNIQKEIDRLNEVAKNLNESLIDLQELGKYEQYIKWPWYI |
| | | WLGFIAGLIAIIMVTIMLCCMTSCCSCLKGCCSCGSCCKFDED |
| | | DSEPVLKGVKLHYT |
| 15 | Artificial | MFVFLFLLPLVSSQCVNLTTRTGIPPGYTNSSTRGVYYPDKVF |
| | Sarbecovirus | RSSVLHLTQDLFLPFFSNVTWFNAINYQGGFKRFDNPVLPFND |
| | spike | GVYFASTEKSNIIRGWIFGTTLDARTQSLLIVNNATNVVIKVCEF |
| | consensus 6 | QFCTDPFLGVYYHNNNKTWVENEFRVYSSANNCTFEYVSQP |
| | | FLMDLEGKQGNFKNLREFVFKNVDGYFKIYSKHTPIDLVRDLP |
| | | RGFSALEPLVDLPIGINITRFQTLLALHRSYLTPGKLESGWTTG |
| | | AAAYYVGYLQPRTFLLSYNENGTITDAVDCALDPLSETKCTLK |
| | | SFTVEKGIYQTSNFRVQPTESIVRFPNITNLCPFGEVFNATRFA |
| | | SVYAWNRKRISNCVADYSVLYNSASFSTFKCYGVSPTKLNDL |
| | | CFTNVYADSFVIRGDEVRQIAPGQTGKIADYNYKLPDDFTGCVI |
| | | AWNSNNLDSKVGGNYNYLYRLFRKSNLKPFERDISTEIYQAGS |
| | | TPCNGVEGFNCYFPLQSYGFQPTNGVGYQPYRVVVLSFELLH |
| | | APATVCGPKKSTNLVKNKCVNFNFNGLTGTGVLTESNKKFLPF |
| | | QQFGRDIADTTDAVRDPQTLEILDITPCSFGGVSVITPGTNTSN |
| | | QVAVLYQDVNCTEVPVAIHADQLTPTWRVYSTGSNVFQTRAG |
| | | CLIGAEHVNNSYECDIPIGAGICASYQTQTNSPRRARSVNSQSI |
| | | IAYTMSLGAENSVAYSNNSIAIPTNFTISVTTEILPVSMTKTSVD |
| | | CTMYICGDSIECSNLLLQYGSFCTQLNRALTGIAVEQDKNTQE |
| | | VFAQVKQIYKTPPIKDFGGFNFSQILPDPSKPSKRSFIEDLLFNK |
| | | VTLADAGFIKQYGDCLGDIAARDLICAQKFNGLTVLPPLLTDEM |
| | | IAQYTSALLAGTITSGWTFGAGAALQIPFAMQMAYRFNGIGVT |
| | | QNVLYENQKLIANQFNSAIGKIQDSLSSTASALGKLQDVVNQN |
| | | AQALNTLVKQLSSNFGAISSVLNDILSRLDPPEAEVQIDRLITGR |
| | | LQSLQTYVTQQLIRAAEIRASANLAATKMSECVLGQSKRVDFC |
| | | GKGYHLMSFPQSAPHGVVFLHVTYVPAQEKNFTTAPAICHEG |
| | | KAHFPREGVFVSNGTHWFVTQRNFYEPQIITTDNTFVSGSCD |
| | | VVIGIVNNTVYDPLQPELDSFKEELDKYFKNHTSPDVDLGDISG |
| | | INASVVNIQKEIDRLNEVAKNLNESLIDLQELGKYEQYIKWPWYI |
| | | WLGFIAGLIAIIMVTIMLCCMTSCCSCLKGCCSCGSCCKFDED |
| | | DSEPVLKGVKLHYT |
| 16 | Artificial | MFVFLFLLPLVSSQCVNLTTRTGIPPGYTNSSTRGVYYPDKVF |
| | Sarbecovirus | RSSILHLTQDLFLPFFSNVTWFNTINYQGGFKKFDNPVLPFND |
| | spike | GVYFASTEKSNIIRGWIFGTTLDNTTQSLLIVNNATNVVIKVCEF |
| | consensus 7 | QFCTDPFLGVYYHNNNKTWVENEFRVYSSANNCTFEYVSQP |
| | | FLMDLEGKQGNFKNLREFVFKNVDGYFKIYSKHTPIDLVRDLP |
| | | RGFSALEPLVDLPIGINITRFQTLLALHRSYLTPGKLESGWTTG |
| | | AAAYYVGYLQQRTFLLSYNENGTITDAVDCSLDPLSETKCTLK |
| | | SLTVEKGIYQTSNFRVQPTISIVRFPNITNLCPFGEVFNATKFAS |
| | | VYAWNRKRISNCVADYSVLYNSTSFSTFKCYGVSPTKLNDLCF |
| | | TNVYADSFVVRGDEVRQIAPGQTGVIADYNYKLPDDFTGC |
| | | VIAWNSVKQDAKTGGNYNYLYRLFRKSKLKPFERDISTEIYQA |
| | | GSTPCNGVVGLNCYYPLERYGFHPTNGVGYQPYRVVVLSFEL |
| | | LNAPATVCGPKLSTTLVKNKCVNFNFNGLTGTGVLTTSKKKFL |
| | | PFQQFGRDISDTTDAVRDPQTLEILDITPCSFGGVSVITPGTNT |
| | | SNQVAVLYQDVNCTEVPMAIHADQLTPTWRVYSTGANVFQTR |
| | | AGCLIGAEHVNNSYECDIPIGAGICASYHTQTNSRSVNSQSIIA |
| | | YTMSLGAENSVAYSNNSIAIPTNFTISVTTEILPVSMTKTSVDCT |
| | | MYICGDSIECSNLLLQYGSFCTQLNRALTGIAVEQDKNTQEVF |
| | | AQVKQIYKTPPIKDFGGFNFSQILPDPSKPSKRSFIEDLLFNKVT |
| | | LADAGFIKQYGDCLGDIAARDLICAQKFNGLTVLPPLLTDEMIA |
| | | QYTSALLAGTITSGWTFGAGAALQIPFAMQMAYRFNGIGVTQN |
| | | VLYENQKLIANQFNSAIGKIQDSLSSTASALGKLQDVVNQNAQ |
| | | ALNTLVKQLSSNFGAISSVLNDILSRLDPPEAEVQIDRLITGRLQ |
| | | SLQTYVTQQLIRAAEIRASANLAATKMSECVLGQSKRVDFCGK |
| | | GYHLMSFPQSAPHGVVFLHVTYVPAQEKNFTTAPAICHEGKA |
| | | HFPREGVFVSNGTHWFVTQRNFYEPQIITTDNTFVSGSCDVVI |
| | | GIVNNTVYDPLQPELDSFKEELDKYFKNHTSPDVDLGDISGINA |
| | | SVVNIQKEIDRLNEVAKNLNESLIDLQELGKYEQYIKWPWYIWL |
| | | GFIAGLIAIIMVTIMLCCMTSCCSCLKGCCSCGSCCKFDEDDSE |
| | | PVLKGVKLHYT |
| 17 | Artificial | MFIFLFFLTLVSSQCVNFDDRTPPPPTQTNSSTRGVYYPDDIF |
| | Sarbecovirus | RSSVLHLTQDLFLPFFSNVTWFHTINYRFDNPVIPFKDGVYFAA |
| | spike | TEKSNVIRGWIFGSTMDNKSQSVIIVNNSTNVVIRVCNFQLCDN |
| | consensus 8 | PFFAVYNKTWTQIESRVYSNAFNCTFEYVSKSFLLDLGEKQGN |
| | | FKHLREFVFKNKDGFLKVYSGYTPIDLVRDLPSGFNALKPIFKL |
| | | PLGINITNFRTLLTAFRPGQSGWGTSAAAYFVGYLKPRTFMLK |
| | | YDENGTITDAVDCSQDPLAELKCTLKSFEVEKGIYQTSNFRVQ |
| | | PTKEVVRFPNITNLCPFGEVFNATTFPSVYAWERKRISNCVAD |
| | | YSVLYNSTSFSTFKCYGVSPTKLNDLCFSNVYADSFVVKGDD |
| | | VRQIAPGQTGVIADYNYKLPDDFTGCVLAWNTRNIDATTGGNY |
| | | NYLYRLFRHSKLKPFERDISNVIYSPGGKPCTPPGFNCYWPLN |
| | | DYGFYPTNGVGYQPYRVVVLSFELLNAPATVCGPKLSTDLVK |
| | | NKCVNFNFNGLTGTGVLTPSSKRFQPFQQFGRDVSDFTDSVR |
| | | DPKTLEILDISPCSFGGVSVITPGTNTSSEVAVLYQDVNCTDVP |
| | | VAIHADQLTPAWRVYSTGNNVFQTQAGCLIGAEHVNNSYECDI |
| | | PIGAGICASYHTVSSLRSTSQKSIVAYTMSLGAENSIAYSNNSIA |
| | | IPTNFSISVTTEVLPVSMTKTSVDCTMYICGDSTECSNLLLQYG |
| | | SFCTQLNRALSGIAVEQDKNTQEVFAQVKQIYKTPPIKDFGGF |
| | | NFSQILPDPSKPSKRSFIEDLLFNKVTLADAGFMKQYGDCLGDI |
| | | NARDLICAQKFNGLTVLPPLLTDEMIAAYTAALVSGTATAGWT |
| | | FGAGAALQIPFAMQMAYRFNGIGVTQNVLYENQKQIANQFNK |
| | | AISQIQESLTTTSTALGKLQDVVNQNAQALNTLVKQLSSNFGAI |
| | | SSVLNDILSRLDPPEAEVQIDRLITGRLQSLQTYVTQQLIRAAEI |
| | | RASANLAATKMSECVLGQSKRVDFCGKGYHLMSFPQAAPHG |
| | | VVFLHVTYVPSQERNFTTAPAICHEGKAHFPREGVFVSNGTH |
| | | WFITQRNFYSPQIITTDNTFVSGSCDVVIGIVNNTVYDPLQPEL |
| | | DSFKEELDKYFKNHTSPDVDLGDISGINASVVNIQKEIDRLNEV |
| | | AKNLNESLIDLQELGKYEQYIKWPWYVWLGFIAGLIAIVMVTIM |
| | | LCCMTSCCSCLKGCCSCGSCCKFDEDDSEPVLKGVKLHYT |
| 18 | Artificial | SNFRVVPSGDVVRFPNITNLCPFGEVFNATKFPSVYAWERKRI |
| | Sarbecovirus | SNCVADYSVLYNSTSFSTFKCYGVSATKLNDLCFSNVYADSFV |
| | RBD | VKGDDVRQIAPGQTGVIADYNYKLPDDFMGCVLAWNTRNIDA |
| | consensus 1 | TSTGNHNYKYRYLRHGKLRPFERDISNVPFSPDGKPCTPPAP |
| | | NCYWPLRGYGFYTTSGIGYQPYRVVVLSFELLNAPATVCGPK |
| | | LSTDLIKNQCVNFNFNGLTGTGVLTPSSKRFQPFQQFGRDVS |
| | | DFTDSVRDPKTSEIL |
| 19 | Artificial | SNFRVAPSKEVVRFPNITNLCPFGEVFNATTFPSVYAWERKRI |
| | Sarbecovirus | SNCVADYSVLYNSTSFSTFKCYGVSATKLNDLCFSNVYADSFV |
| | RBD | VKGDDVRQIAPGQTGVIADYNYKLPDDFTGCVLAWNTRNIDAT |
| | consensus 2 | QTGNYNYKYRSLRHGKLRPFERDISNVPFSPDGKPCTPPAFN |
| | | CYWPLNDYGYTTNGIGYQPYRVVVLSFELLNAPATVCGPKLS |
| | | TDLIKNQCVNFNFNGLTGTGVLTPSSRFQPFQQFGRDVSDFT |
| | | DSVRDPKTSEIL |
| 20 | Artificial | SNFRVQPTISIVRFPNITNLCPFGEVFNATRFASVYAWNRKRIS |
| | Sarbecovirus | NCVADYSVLYNSTSFSTFKCYGVSPTKLNDLCFTNVYADSFVV |
| | RBD | RGDEVRQIAPGQTGVIADYNYKLPDDFTGCVIAWNSVKQDAL |
| | consensus 3 | TGGNYNYLYRLFRKSKLKPFERDISTEIYQAGSTPCNGVVGLN |
| | | CYYPLQRYGFHPTTGVGYQPYRVVVLSFELLNAPATVCGPKK |
| | | STNLVKNKCVNFNFNGLTGTGVLTESNKKFLPFQQFGRDIADT |
| | | TDAVRDPQTLEIL |
| 21 | Artificial | SNFRVQPTISIVRFPNITNLCPFGEVFNATKFASVYAWNRKRIS |
| | Sarbecovirus | NCVADYSVLYNSTSFSTFKCYGVSPTKLNDLCFTNVYADSFVV |
| | RBD | RGDEVRQIAPGQTGVIADYNYKLPDDFTGCVIAWNSVKQDAK |
| | consensus 4 | TGGNYNYLYRLFRKSKLKPFERDISTEIYQAGSTPCNGVVGLN |
| | | CYYPLERYGFHPTNGVGYQPYRVVVLSFELLNAPATVCGPKL |
| | | STTLVKNKCVNFNFNGLTGTGVLTTSKKKFLPFQQFGRDISDT |
| | | TDAVRDPQTLEIL |
| 22 | Artificial | SNFRVQPTKEVVRFPNITNLCPFGEVFNATTFPSVYAWERKRI |
| | Sarbecovirus | SNCVADYSVLYNSTSFSTFKCYGVSPTKLNDLCFSNVYADSFV |
| | RBD | VKGDDVRQIAPGQTGVIADYNYKLPDDFTGCVLAWNTRNIDAT |
| | consensus 5 | TGGNYNYLYRLFRHSKLKPFERDISNVIYSPGGKPCTPPGFNC |
| | | YWPLNDYGFYPTNGVGYQPYRVVVLSFELLNAPATVCGPKLS |
| | | TDLVKNKCVNFNFNGLTGTGVLTPSSKRFQPFQQFGRDVSDF |
| | | TDSVRDPKTLEIL |
| 23 | Coronavirus | MKLLVLVFATLVSSYTIEKCTDFDDRTPPSNTQFLSSHRGVYY |
| | BtRs- | PDDIFRSNVLHLVQDHFLPFDSNVTRFITFGLNFDNP |
| | BetaCoV/ | IIPFRDGVYFAATEKSNVIRGWVFGSTMNNKSQSVIIMNNSTNL |
| | YN2018B Spike | VIRACNFELCDNPFFVVLRSNNTQIPSYIFNNAFNC |
| | protein | TFEYVSKDFNLDIGEKPGNFKDLREFVFRNKDGFLHVYSGYQ |
| | (Genbank: | PISAASGLPTGFNALKPIFKLPLGINITNFRTLLTAFP |
| | QDF43825) | PNPGYWGTSAAAYFVGYLKPTTFMLKYDENGTITDAVDCSQN |
| | | PLAELKCSVKSFEIDKGIYQTSNFRVAPSKEVVRFPNI |
| | | TNLCPFGEVFNATTFPSVYAWERKRISNCVADYSVLYNSTSFS |
| | | TFKCYGVSATKLNDLCFSNVYADSFVVKGDDVRQIAP |
| | | GQTGVIADYNYKLPDDFMGCVLAWNTRNIDATSTGNYNYKYR |
| | | SLRHGKLRPFERDISNVPFSPDGKPCTPPAFNCYWPLN |
| | | DYGFFTTNGIGYQPYRVVVLSFELLNAPATVCGPKLSTDLIKN |
| | | QCVNFNFNGLTGTGVLTPSSKRFQPFQQFGRDVSDFT |
| | | DSVRDPKTSEILDISPCSFGGVSVITPGTNTSSEVAVLYQDVNC |
| | | TDVPVAIHADQLTPAWRIYSTGNNVFQTQAGCLIGA |
| | | EHVDTSYECDIPIGAGICASYHTVSSLRSTSQKSIVAYTMSLGA |
| | | DSSIAYSNNTIAIPTNFSISITTEVMPVSMAKTSVD |
| | | CNMYICGDSTECANLLLQYGSFCTQLNRALSGIAVEQDRNTR |
| | | EVFAQVKQMYKTPTLKDFGGFNFSQILPDPLKPTKRSF |
| | | IEDLLFNKVTLADAGFMKQYGECLGDINARDLICAQKFNGLTVL |
| | | PPLLTDDMIAAYTAALVSGTATAGWTFGAGAALQIP |
| | | FAMQMAYRFNGIGVTQNVLYENQKQIANQFNKAISQIQESLTT |
| | | TSTALGKLQDVVNQNAQALNTLVKQLSSNFGAISSVL |
| | | NDILSRLDKVEAEVQIDRLITGRLQSLQTYVTQQLIRAAEIRASA |
| | | NLAATKMSECVLGQSKRVDFCGKGYHLMSFPQAAP |
| | | HGVVFLHVTYVPSQERNFTTAPAICHEGKAYFPREGVFVFNG |
| | | TSWFITQRNFFSPQIITTDNTFVSGSCDVVIGIINNTV |
| | | YDPLQPELDSFKEELDKYFKNHTSPDVDLGDISGINASVVNIQK |
| | | EIDRLNEVAKNLNESLIDLQELGKYEQYIKWPWYVW |
| | | LGFIAGLIAIVMVTILLCCMTSCCSCLKGACSCGSCCKFDEDDS |
| | | EPVLKGVKLHYT |
| 24 | Bat SARS-like | MKLLVLVFATLVSSYTIEKCLDFDDRTPPANTQFLSSHRGVYY |
| | coronavirus | PDDIFRSNVLHLVQDHFLPFDSNVTRFITFGLNFDNP |
| | Spike protein | IIPFRDGIYFAATEKSNVIRGWVFGSTMNNKSQSVIIMNNSTNL |
| | (Genbank: | VIRACNFELCDNPFFVVLKSNNTQIPSYIFNNAFNC |
| | ATO98218) | TFEYVSKDFNLDLGEKPGNFKDLREFVFRNKDGFLHVYSGYQ |
| | | PISAASGLPTGFNALKPIFKLPLGINITNFRTLLTAFP |
| | | PRPDYWGTSAAAYFVGYLKPTTFMLKYDENGTITDAVDCSQN |
| | | PLAELKCSVKSFEIDKGIYQTSNFRVAPSKEVVRFPNI |
| | | TNLCPFGEVFNATTFPSVYAWERKRISNCVADYSVLYNSTSFS |
| | | TFKCYGVSATKLNDLCFSNVYADSFVVKGDDVRQIAP |
| | | GQTGVIADYNYKLPDDFMGCVLAWNTRNIDATSTGNYNYKYR |
| | | SLRHGKLRPFERDISNVPFSPDGKPCTPPAFNCYWPLN |
| | | DYGFFTTNGIGYQPYRVVVLSFELLNAPATVCGPKLSTDLIKN |
| | | QCVNFNFNGLTGTGVLTSSSKRFQPFQQFGRDVSDFT |
| | | DSVRDPKTSEILDISPCSFGGVSVITPGTNTSSEVAVLYQDVNC |
| | | TDVPVAIHADQLTPAWRIYSTGNNVFQTQAGCLIGA |
| | | EHVDTSYECDIPIGAGICASYHTVSSLRSTSQKSIVAYTMSLGA |
| | | DSSIVYSNNTIAIPTNFSISITTEVMPVSMAKTSVD |
| | | CNMYICGDSTECANLLLQYGSFCTQLNRALSGIAVEQDRNTR |
| | | EVFAQVKQMYKTPTLKDFGGFNFSQILPDPLKPTKRSF |
| | | IEDLLFNKVTLADAGFMKQYGECLGDINARDLICAQKFNGLTVL |
| | | PPLLTDDMIAAYTAALVSGTATAGWTFGAGAALQIP |
| | | FAMQMAYRFNGIGVTQNVLYENQKQIANQFNKAISQIQESLTT |
| | | TSTALGKLQDVVNQNAQALNTLVKQLSSNFGAISSVL |
| | | NDILSRLDKVEAEVQIDRLITGRLQSLQTYVTQQLIRAAEIRASA |
| | | NLAATKMSECVLGQSKRVDFCGKGYHLMSFPQAAP |
| | | HGVVFLHVTYVPSQERNFTTAPAICHEGKAYFPREGVFVFNG |
| | | TSWFITQRNFFSPQIITTDNTFVSGNCDVVIGIINNTV |
| | | YDPLQPELDSFKEELDKYFKNHTSPDVDLGDISGINASVVNIQK |
| | | EIDRLNEVAKNLNESLIDLQELGKYEQYIKWPWYVW |
| | | LGFIAGLIAIVMVTILLCCMTSCCSCLKGACSCGSCCKFDEDDS |
| | | EPVLKGVKLHYT |
| 25 | Bat SARS-like | MKLLVLVFATLVSSYTIEKCLDFDDRTPPANTQFLSSHRGVYY |
| | coronavirus | PDDIFRSNVLHLVQDHFLPFDSNVTRFITFGLNFDNP |
| | Spike protein | IIPFRDGIYFAATEKSNVIRGWVFGSTMNNKSQSVIIMNNSTNL |
| | (Genbank: | VIRACNFELCDNPFFVVLKSNNTQIPSYIFNNAFNC |
| | ATO98231) | TFEYVSKDFNLDLGEKPGNFKDLREFVFRNKDGFLHVYSGYQ |
| | | PISAASGLPTGFNALKPIFKLPLGINITNFRTLLTAFP |
| | | PRPDYWGTSAAAYFVGYLKPTTFMLKYDENGTITDAVDCSQN |
| | | PLAELKCSVKSFEIDKGIYQTSNFRVAPSKEVVRFPNI |
| | | TNLCPFGEVFNATTFPSVYAWERKRISNCVADYSVLYNSTSFS |
| | | TFKCYGVSATKLNDLCFSNVYADSFVVKGDDVRQIAP |
| | | GQTGVIADYNYKLPDDFMGCVLAWNTRNIDATSTGNYNYKYR |
| | | SLRHGKLRPFERDISNVPFSPDGKPCTPPAFNCYWPLN |
| | | DYGFFTTNGIGYQPYRVVVLSFELLNAPATVCGPKLSTDLIKN |
| | | QCVNFNFNGLTGTGVLTSSSKRFQPFQQFGRDVSDFT |
| | | DSVRDPKTSEILDISPCSFGGVSVITPGTNTSSEVAVLYQDVNC |
| | | TDVPVAIHADQLTPAWRIYSTGNNVFQTQAGCLIGA |
| | | EHVDTSYECDIPIGAGICASYHTVSSLRSTSQKSIVAYTMSLGA |
| | | DSSIVYSNNTIAIPTNFSISITTEVMPVSMAKTSVD |
| | | CNMYICGDSTECANLLLQYGSFCTQLNRALSGIAVEQDRNTR |
| | | EVFAQVKQMYKTPTLKDFGGFNFSQILPDPLKPTKRSF |
| | | IEDLLFNKVTLADAGFMKQYGECLGDINARDLICAQKFNGLTVL |
| | | PPLLTDDMIAAYTAALVSGTATAGWTFGAGAALQIP |
| | | FAMQMAYRFNGIGVTQNVLYENQKQIANQFNKAISQIQESLTT |
| | | TSTALGKLQDVVNQNAQALNTLVKQLSSNFGAISSVL |
| | | NDILSRLDKVEAEVQIDRLITGRLQSLQTYVTQQLIRAAEIRASA |
| | | NLAATKMSECVLGQSKRVDFCGKGYHLMSFPQAAP |
| | | HGVVFLHVTYVPSQERNFTTAPAICHEGKAYFPREGVFVFNG |
| | | TSWFITQRNFFSPQIITTDNTFVSGNCDVVIGIINNTV |
| | | YDPLQPELDSFKEELDKYFKNHTSPDVDLGDISGINASVVNIQK |
| | | EIDRLNEVAKNLNESLIDLQELGKYEQYIKWPWYVW |
| | | LGFIAGLIAIVMVTILLCCMTSCCSCLKGACSCGSCCKFDEDDP |
| | | EPVLKGVKLHYT |
| 26 | Bat SARS-like | MKLLVLVFATLVSSYTIEKCLDFDDRTPPANTQFLSSHRGVYY |
| | coronavirus | PDDIFRSNVLHLVQDHFLPFDSNVTRFITFGLNFDNP |
| | RsSHC014 | IIPFRDGIYFAATEKSNVIRGWVFGSTMNNKSQSVIIMNNSTNL |
| | Spike protein | VIRACNFELCDNPFFVVLKSNNTQIPSYIFNNAFNC |
| | (Genbank: | TFEYVSKDFNLDLGEKPGNFKDLREFVFRNKDGFLHVYSGYQ |
| | AGZ48806) | PISAASGLPTGFNALKPIFKLPLGINITNFRTLLTAFP |
| | | PRPDYWGTSAAAYFVGYLKPTTFMLKYDENGTITDAVDCSQN |
| | | PLAELKCSVKSFEIDKGIYQTSNFRVAPSKEVVRFPNI |
| | | TNLCPFGEVFNATTFPSVYAWERKRISNCVADYSVLYNSTSFS |
| | | TFKCYGVSATKLNDLCFSNVYADSFVVKGDDVRQIAP |
| | | GQTGVIADYNYKLPDDFLGCVLAWNTNSKDSSTSGNYNYLYR |
| | | WVRRSKLNPYERDLSNDIYSPGGQSCSAVGPNCYNPLR |
| | | PYGFFTTAGVGHQPYRVVVLSFELLNAPATVCGPKLSTDLIKN |
| | | QCVNFNFNGLTGTGVLTPSSKRFQPFQQFGRDVSDFT |
| | | DSVRDPKTSEILDISPCSFGGVSVITPGTNTSSEVAVLYQDVNC |
| | | TDVPVAIHADQLTPSWRVYSTGNNVFQTQAGCLIGA |
| | | EHVDTSYECDIPIGAGICASYHTVSSLRSTSQKSIVAYTMSLGA |
| | | DSSIAYSNNTIAIPTNFSISITTEVMPVSMAKTSVD |
| | | CNMYICGDSTECANLLLQYGSFCTQLNRALSGIAVEQDRNTR |
| | | EVFAQVKQMYKTPTLKDFGGFNFSQILPDPLKPTKRSF |
| | | IEDLLFNKVTLADAGFMKQYGECLGDINARDLICAQKFNGLTVL |
| | | PPLLTDDMIAAYTAALVSGTATAGWTFGAGAALQIP |
| | | FAMQMAYRFNGIGVTQNVLYENQKQIANQFNKAISQIQESLTT |
| | | TSTALGKLQDVVNQNAQALNTLVKQLSSNFGAISSVL |
| | | NDILSRLDKVEAEVQIDRLITGRLQSLQTYVTQQLIRAAEIRASA |
| | | NLAATKMSECVLGQSKRVDFCGKGYHLMSFPQAAP |
| | | HGVVFLHVTYVPSQERNFTTAPAICHEGKAYFPREGVFVFNG |
| | | TSWFITQRNFFSPQIITTDNTFVSGSCDVVIGIINNTV |
| | | YDPLQPELDSFKEELDKYFKNHTSPDVDLGDISGINASVVNIQK |
| | | EIDRLNEVAKNLNESLIDLQELGKYEQYIKWPWYVW |
| | | LGFIAGLIAIVMVTILLCCMTSCCSCLKGACSCGSCCKFDEDDS |
| | | EPVLKGVKLHYT |
| 27 | Bat SARS-like | MKLLVLVFATLVSSYTIEKCLDFDDRTPPANTQFLSSHRGVYY |
| | coronavirus | PDDIFRSNVLHLVQDHFLPFDSNVTRFITFGLNFDNP |
| | Spike protein | IIPFRDGIYFAATEKSNVIRGWVFGSTMNNKSQSVIIMNNSTNL |
| | (Genbank: | VIRACNFELCDNPFFVVLKSNNTQIPSYIFNNAFNC |
| | ATO98132) | TFEYVSKDFNLDLGEKPGNFKDLREFVFRNKDGFLHVYSGYQ |
| | | PISAASGLPTGFNALKPIFKLPLGINITNFRTLLTAFP |
| | | PRPDYWGTSAAAYFVGYLKPTTFMLKYDENGTITDAVDCSQN |
| | | PLAELKCSVKSFEIDKGIYQTSNFRVAPSKEVVRFPNI |
| | | TNLCPFGEVFNATTFPSVYAWERKRISNCVADYSILYNSTSFS |
| | | TFKCYGVSATKLNDLCFSNVYADSFVVKGDDVRQIAP |
| | | GQTGVIADYNYKLPDDFLGCVLAWNTNSKDSSTSGNYNYLYR |
| | | WVRRSKLNPYERDLSNDIYSPGGQSCSAVGPNCYNPLR |
| | | PYGFFTTAGVGHQPYRVVVLSFELLNAPATVCGPKLSTDLIKN |
| | | QCVNFNFNGLTGTGVLTPSSKRFQPFQQFGRDVSDFT |
| | | DSVRDPKTSEILDISPCSFGGVSVITPGTNTSSEVAVLYQDVNC |
| | | TDVPVAIHADQLTPSWRVYSTGNNVFQTQAGCLIGA |
| | | EHVDTSYECDIPIGAGICASYHTVSSLRSTSQKSIVAYTMSLGA |
| | | DSSIAYSNNTIAIPTNFSISITTEVMPVSMAKTSVD |
| | | CNMYICGDSTECANLLLQYGSFCTQLNRALSGIAVEQDRNTR |
| | | EVFAQVKQMYKTPTLKDFGGFNFSQILPDPLKPTKRSF |
| | | IEDLLFNKVTLADAGFMKQYGECLGDINARDLICAQKFNGLTVL |
| | | PPLLTDDMIAAYTAALVSGTATAGWTFGAGAALQIP |
| | | FAMQMAYRFNGIGVTQNVLYENQKQIANQFNKAISQIQESLTT |
| | | TSTALGKLQDVVNQNAQALNTLVKQLSSNFGAISSVL |
| | | NDILSRLDKVEAEVQIDRLITGRLQSLQTYVTQQLIRAAEIRASA |
| | | NLAATKMSECVLGQSKRVDFCGKGYHLMSFPQAAP |
| | | HGVVFLHVTYVPSQERNFTTAPAICHEGKAYFPREGVFVFNG |
| | | TSWFITQRNFFSPQIITTDNTFVSGSCDVVIGIINNTV |
| | | YDPLQPELDSFKEELDKYFKNHTSPDVDLGDISGINASVVNIQK |
| | | EIDRLNEVAKNLNESLIDLQELGKYEQYIKWPWYVW |
| | | LGFIAGLIAIVMVTILLCCMTSCCSCLKGACSCGSCCKFDEDDS |
| | | EPVLKGVKLHYT |
| 28 | Bat SARS-like | MKLLVLVFATLVSSYTIEKCLDFDDRTPPANTQFLSSHRGVYY |
| | coronavirus | PDDIFRSNVLHLVQDHFLPFDSNVTRFITFGLNFDNP |
| | Rs3367 | IIPFKDGIYFAATEKSNVIRGWVFGSTMNNKSQSVIIMNNSTNL |
| | Spike protein | VIRACNFELCDNPFFVVLKSNNTQIPSYIFNNAFNC |
| | (Genbank: | TFEYVSKDFNLDLGEKPGNFKDLREFVFRNKDGFLHVYSGYQ |
| | AGZ48818) | PISAASGLPTGFNALKPIFKLPLGINITNFRTLLTAFP |
| | | PRPDYWGTSAAAYFVGYLKPTTFMLKYDENGTITDAVDCSQN |
| | | PLAELKCSVKSFEIDKGIYQTSNFRVAPSKEVVRFPNI |
| | | TNLCPFGEVFNATTFPSVYAWERKRISNCVADYSVLYNSTSFS |
| | | TFKCYGVSATKLNDLCFSNVYADSFVVKGDDVRQIAP |
| | | GQTGVIADYNYKLPDDFTGCVLAWNTRNIDATQTGNYNYKYR |
| | | SLRHGKLRPFERDISNVPFSPDGKPCTPPAFNCYWPLN |
| | | DYGFYITNGIGYQPYRVVVLSFELLNAPATVCGPKLSTDLIKNQ |
| | | CVNFNFNGLTGTGVLTPSSKRFQPFQQFGRDVSDFT |
| | | DSVRDPKTSEILDISPCSFGGVSVITPGTNTSSEVAVLYQDVNC |
| | | TDVPVAIHADQLTPSWRVYSTGNNVFQTQAGCLIGA |
| | | EHVDTSYECDIPIGAGICASYHTVSSLRSTSQKSIVAYTMSLGA |
| | | DSSIAYSNNTIAIPTNFSISITTEVMPVSMAKTSVD |
| | | CNMYICGDSTECANLLLQYGSFCTQLNRALSGIAVEQDRNTR |
| | | EVFAQVKQMYKTPTLKDFGGFNFSQILPDPLKPTKRSF |
| | | IEDLLFNKVTLADAGFMKQYGECLGDINARDLICAQKFNGLTVL |
| | | PPLLTDDMIAAYTAALVSGTATAGWTFGAGAALQIP |
| | | FAMQMAYRFNGIGVTQNVLYENQKQIANQFNKAISQIQESLTT |
| | | TSTALGKLQDVVNQNAQALNTLVKQLSSNFGAISSVL |
| | | NDILSRLDKVEAEVQIDRLITGRLQSLQTYVTQQLIRAAEIRASA |
| | | NLAATKMSECVLGQSKRVDFCGKGYHLMSFPQAAP |
| | | HGVVFLHVTYVPSQERNFTTAPAICHEGKAYFPREGVFVFNG |
| | | TSWFITQRNFFSPQIITTDNTFVSGSCDVVIGIINNTV |
| | | YDPLQPELDSFKEELDKYFKNHTSPDVDLGDISGINASVVNIQK |
| | | EINRLNEVAKNLNESLIDLQELGKYEQYIKWPWYVW |
| | | LGFIAGLIAIVMVTILLCCMTSCCSCLKGACSCGSCCKFDEDDS |
| | | EPVLKGVKLHYT |
| 29 | Bat SARS-like | MKLLVLVFATLVSSYTIEKCLDFDDRTPPANTQFLSSHRGVYY |
| | coronavirus | PDDIFRSNVLHLVQDHFLPFDSNVTRFITFGLNFDNP |
| | WIV1 | IIPFKDGIYFAATEKSNVIRGWVFGSTMNNKSQSVIIMNNSTNL |
| | Spike protein | VIRACNFELCDNPFFVVLKSNNTQIPSYIFNNAFNC |
| | (Genbank: | TFEYVSKDFNLDLGEKPGNFKDLREFVFRNKDGFLHVYSGYQ |
| | AGZ48828) | PISAASGLPTGFNALKPIFKLPLGINITNFRTLLTAFP |
| | | PRPDYWGTSAAAYFVGYLKPTTFMLKYDENGTITDAVDCSQN |
| | | PLAELKCSVKSFEIDKGIYQTSNFRVAPSKEVVRFPNI |
| | | TNLCPFGEVFNATTFPSVYAWERKRISNCVADYSVLYNSTSFS |
| | | TFKCYGVSATKLNDLCFSNVYADSFVVKGDDVRQIAP |
| | | GQTGVIADYNYKLPDDFTGCVLAWNTRNIDATQTGNYNYKYR |
| | | SLRHGKLRPFERDISNVPFSPDGKPCTPPAFNCYWPLN |
| | | DYGFYITNGIGYQPYRVVVLSFELLNAPATVCGPKLSTDLIKNQ |
| | | CVNFNFNGLTGTGVLTPSSKRFQPFQQFGRDVSDFT |
| | | DSVRDPKTSEILDISPCSFGGVSVITPGTNTSSEVAVLYQDVNC |
| | | TDVPVAIHADQLTPSWRVHSTGNNVFQTQAGCLIGA |
| | | EHVDTSYECDIPIGAGICASYHTVSSLRSTSQKSIVAYTMSLGA |
| | | DSSIAYSNNTIAIPTNFSISITTEVMPVSMAKTSVD |
| | | CNMYICGDSTECANLLLQYGSFCTQLNRALSGIAVEQDRNTR |
| | | EVFAQVKQMYKTPTLKDFGGFNFSQILPDPLKPTKRSF |
| | | IEDLLFNKVTLADAGFMKQYGECLGDINARDLICAQKFNGLTVL |
| | | PPLLTDDMIAAYTAALVSGTATAGWTFGAGAALQIP |
| | | FAMQMAYRFNGIGVTQNVLYENQKQIANQFNKAISQIQESLTT |
| | | TSTALGKLQDVVNQNAQALNTLVKQLSSNFGAISSVL |
| | | NDILSRLDKVEAEVQIDRLITGRLQSLQTYVTQQLIRAAEIRASA |
| | | NLAATKMSECVLGQSKRVDFCGKGYHLMSFPQAAP |
| | | HGVVFLHVTYVPSQERNFTTAPAICHEGKAYFPREGVFVFNG |
| | | TSWFITQRNFFSPQIITTDNTFVSGSCDVVIGIINNTV |
| | | YDPLQPELDSFKEELDKYFKNHTSPDVDLGDISGINASVVNIQK |
| | | EIDRLNEVAKNLNESLIDLQELGKYEQYIKWPWYVW |
| | | LGFIAGLIAIVMVTILLCCMTSCCSCLKGACSCGSCCKFDEDDS |
| | | EPVLKGVKLHYT |
| 30 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Tor2 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (Genbank: | DVVRDLPSGFNTLKPIFKLPLGINITNFRAILTAFSP |
| | YP_009825051) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCAFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 31 | Bat SARS-like | MFIFLFFLTLTSGSDLESCTTFDDVQAPNYPQHSSSRRGVYYP |
| | coronavirus | DEIFRSDTLYLTQDLFLPFYSNVTGFHTINHRFDNPV |
| | Spike protein | IPFKDGVYFAATEKSNVVRGWVFGSTMNNKSQSVIIINNSTNV |
| | (Genbank: | VIRACNFELCDNPFFAVSKPTGTQTHTMIFDNAFNCT |
| | ATO98157) | FEYISDSFSLDVAEKSGNFKHLREFVFKNKDGFLYVYKGYQPI |
| | | DVVRDLPSGFNILKPIFKLPLGINITNFRAILTAFLP |
| | | AQDTWGTSAAAYFVGYLKPATFMLKYDENGTITDAVDCSQNP |
| | | LAELKCSVKSFEIDKGIYQTSNFRVAPSKEVVRFPNIT |
| | | NLCPFGEVFNATTFPSVYAWERKRISNCVADYSVLYNSTSFST |
| | | FKCYGVSATKLNDLCFSNVYADSFVVKGDDVRQIAPG |
| | | QTGVIADYNYKLPDDFLGCVLAWNTNSKDSSTSGNYNYLYRW |
| | | VRRSKLNPYERDLSNDIYSPGGQSCSAIGPNCYNPLRP |
| | | YGFFTTAGVGHQPYRVVVLSFELLNAPATVCGPKLSTDLIKNQ |
| | | CVNFNFNGLTGTGVLTSSSKRFQPFQQFGRDVSDFTD |
| | | SVRDPKTSEILDISPCSFGGVSVITPGTNTSSEVAVLYQDVNCT |
| | | DVPVAIHADQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAVEQDRNTREV |
| | | FAQVKQMYKTPALKDFGGFNFSQILPDPLKPTKRSFI |
| | | EDLLFNKVTLADAGFMKQYGECLGDVNARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTCVPSQERNFTTAPAICHEGKAYFPREGVFVFNGT |
| | | SWFITQRNFFSPQIITTDNTFVSGSCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 32 | SARS-like | MFIFLFFLTLTSGSDLESCTTFDDVQAPNYPQHSSSRRGVYYP |
| | coronavirus | DEIFRSDTLYLTQDLFLPFYSNVTGFHTINHRFDNPV |
| | WIV16 | IPFKDGVYFAATEKSNVVRGWVFGSTMNNKSQSVIIINNSTNV |
| | Spike protein | VIRACNFELCDNPFFAVSKPTGTQTHTMIFDNAFNCT |
| | (Genbank: | FEYISDSFSLDVAEKSGNFKHLREFVFKNKDGFLYVYKGYQPI |
| | ALK02457) | DVVRDLPSGFNILKPIFKLPLGINITNFRAILTAFLP |
| | | AQDTWGTSAAAYFVGYLKPATFMLKYDENGTITDAVDCSQNP |
| | | LAELKCSVKSFEIDKGIYQTSNFRVAPSKEVVRFPNIT |
| | | NLCPFGEVFNATTFPSVYAWERKRISNCVADYSVLYNSTSFST |
| | | FKCYGVSATKLNDLCFSNVYADSFVVKGDDVRQIAPG |
| | | QTGVIADYNYKLPDDFTGCVLAWNTRNIDATQTGNYNYKYRS |
| | | LRHGKLRPFERDISNVPFSPDGKPCTPPAFNCYWPLND |
| | | YGFYITNGIGYQPYRVVVLSFELLNAPATVCGPKLSTDLIKNQC |
| | | VNFNFNGLTGTGVLTPSSKRFQPFQQFGRDVLDFTD |
| | | SVRDPKTSEILDISPCSFGGVSVITPGTNTSSEVAVLYQDVNCT |
| | | DVPVAIHADQLTPSWRVYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAVEQDRNTREV |
| | | FAQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGSCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 33 | Bat SARS-like | MFIFLFFLTLTSGSDLESCTTFDDVQAPNYPQHSSSRRGVYYP |
| | coronavirus | DEIFRSDTLYLTQDLFLPFYSNVTGFHTINHRFDNPV |
| | Spike protein | IPFKDGVYFAATEKSNVVRGWVFGSTMNNKSQSVIIINNSTNV |
| | (Genbank: | VIRACNFELCDNPFFAVSKPTGTQTHTMIFDNAFNCT |
| | ATO98205) | FEYISDSFSLDVAEKSGNFKHLREFVFKNKDGFLYVYKGYQPI |
| | | DVVRDLPSGFNILKPIFKLPLGINITNFRAILTAFLP |
| | | AQDTWGTSAAAYFVGYLKPATFMLKYDENGTITDAVDCSQNP |
| | | LAELKCSVKSFEIDKGIYQTSNFRVAPSKEVVRFPNIT |
| | | NLCPFGEVFNATTFPSVYAWERKRISNCVADYSVLYNSTSFST |
| | | FKCYGVSATKLNDLCFSNVYADSFVVKGDDVRQIAPG |
| | | QTGVIADYNYKLPDDFTGCVLAWNTRNIDATQTGNYNYKYRS |
| | | LRHGKLRPFERDISNVPFSPDGKPCTPPAFNCYWPLND |
| | | YGFYITNGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNTSSEVAVLYQDVNCT |
| | | DVPVAIHADQLTPSWRVYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAVEQDRNTREV |
| | | FAQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGSCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 34 | Rhinolophus | MFLTCFILSFSLFCVSGDSIDTCETFDDVSPPQQNLVSSSKRG |
| | affinis | VYYPDDIYRSDVHHLVQDLFLPFNSNVVGLMSFNYRF |
| | coronavirus | DNPIIPFKDGVYFAATEKSNVVRGWVFGSTMNNKSQSVIIMNN |
| | Spike protein | STNVVIRACNFQLCDNPFFAVIRPTSQQIETILFENA |
| | (Genbank: | FNCTFEYVSDSFLMDVGEKPGNFKHLREFIFKNKDGFLNIYSG |
| | AHX37558) | FQNIDVANGLPSGFSLLKPLLKLPLGLNITNFRVLLT |
| | | AFIPNIGTWGTSPVAYFVGYLKPTTFMLKYDYNGTIVDAVDCS |
| | | QHPLAELKCSVKSFEIDKGIYQTSNFRVSPSKEVVRF |
| | | PNITNLCPFGEVFNATTFPSVYAWERKRISNCVADYSVLYNST |
| | | SFSTFKCYGVSAIKLNDLCFSNVYADSFVVKGDDVRQ |
| | | IAPGQTGVIADYNYKLPDDFMGCVLAWNTRNIDATSSGNFNYK |
| | | YRSLRHGKLRPFERDISNVPFSPDGKPCTPPAFNCYW |
| | | PLNDYGFYTTNGIGYQPYRVVVLSFELLNAPATVCGPKLSTDLI |
| | | TNQCVNFNFNGLTGTGVLTPSLKRFQPFQQFGRDFS |
| | | DFTDSVRDPKTLEVLDISPCSFGGVSVITPGTNISSEVAVLYQD |
| | | VNCTDVPTAIHADQLTPAWRIYSAGVNVFQTQAGCL |
| | | IGAEHVNASYECDIPIGAGICASYHTASLLRNTDQKSIVAYTMS |
| | | LGAENSIAYANNSIAIPTNFSISITTEVMPVSMAKT |
| | | SVDCTMYICGDSQECSNLLLQYGSFCTQLNRALSGIAVEQDK |
| | | NTQEVFAQVKQIYKTPAIKDFGGFNFSQILPDPSKPTK |
| | | RSFIEDLLFNKVTLADAGFMKQYGECLGDISARDLICAQKFNGL |
| | | TVLPPLLTDEMIAAYTAALVSGTATAGWTFGAGAAL |
| | | QIPFAMQMAYRFNGIGVTQNVLYENQKQIANQFNKAISQIQES |
| | | LTTTSTALGKLQDVVNQNAQALNTLVKQLSSNFGAIS |
| | | SVLNDILSRLDKVEAEVQIDRLITGRLQSLQTYVTQQLIRAAEIR |
| | | ASANLAATKMSECVLGQSKRVDFCGKGYHLMSFPQ |
| | | AAPHGVVFLHVTYVPSQERNFTTAPAICHEGKAYFPREGVFVS |
| | | NGTSWFITQRNFFSPQLITTDNTFVSGNCDVVIGIIN |
| | | NTVYDPLQPELDSFKEELDKYFKNHTSPDVDLGDISRINASVV |
| | | NIQKEIDRLNEVAKNLNESLIDLQELGKYEQYIKWPW |
| | | YVWLGFIAGLIAIVMATILLCCMTSCCSCLKGACSCGSCCKFDE |
| | | DDSEPVLKGVKLHYT |
| 35 | Rhinolophus | MFLTCFILIFSLFCVSGDSIDTCETFDDVSPPQQNLVSSSKRGV |
| | affinis | YYPDDIYRSDAHHLVQDLFLPFNSNVVGLMSFNYRF |
| | coronavirus | DNPIIPFKDGVYFAATEKSNVVRGWVFGSTMNNKSQSAIIMNN |
| | Spike protein | STNVVIRACNFQLCDNPFFAVIRPTSQQIETILFENA |
| | (Genbank: | FNCTFEYVSDSFLMDVGEKPGNFKHLREFIFKNKDGFLYIYSG |
| | AHX37569) | FQNIDVANGLPSGFSLLKPLLKLPLGLNITNFRVLLT |
| | | AFIPNIGTWGTSPVAYFVGYLKPTTFMLKYDYNGTIVDAVDCS |
| | | QHPLAELKCSVKSFDIDKGIYQTSNFRVSPSREVVRF |
| | | PNITNLCPFGEVFNATTFPSVYAWERKRISNCVADYSVLYNST |
| | | SFSTFKCYGVSAIKLNDLCFSNVYADSFVVKGDDVRQ |
| | | IAPGQTGVIADYNYKLPDDFMGCVLAWNTRNIDATSSGNFHYK |
| | | YRSLRHGKLRPFERDISNVPFSPDGKPCTPPAFNCYW |
| | | PLNDYGFYTTNGIGYQPYRVVVLSFELLNAPATVCGPKLSTDLI |
| | | TNQCVNFNFNGLTGTGVLTPSSKRFQPFQQFGRDVS |
| | | DFTDSVRDPKTLEVLDISPCSFGGVSVITPGTNISSEVAVLYQD |
| | | VNCTDVPTAIHADQLTPAWRIYSAGENVFQTQAGCL |
| | | IGAEHVNASYECDIPIGAGICASYHTASLLRNTGQKSIVAYTMS |
| | | LGAENSIAYANNSIAIPTNFSISITTEVMPVSMAKT |
| | | SVDCTMYICGDSQECSNLLLQYGSFCTQLNRALSGIAVEQDS |
| | | NTQEVFAQVKQIYKTPAIKDFGGFNFSQILPDPSKPTK |
| | | RSFIEDLLFNKVTLADAGFMKQYGECLGDISARDLICAQKFNGL |
| | | TVLPPLLTDEMIAAYTAALVSGTATAGWTFGAGAAL |
| | | QIPFAMQMAYRFNGIGVTQNVLYENQKQIANQFNKAISQIQES |
| | | LTTTSTALGKLQDVVNQNAQALNTLVKQLSSNFGAIS |
| | | SVLNDILSRLDKVEAEVQIDRLITGRLQSLQTYVTQQLIRAAEIR |
| | | ASANLAATKMSECVLGQSKRVDFCGKGYHLMSFPQ |
| | | AAPHGVVFLHVTYVPSQERNFTTAPAICHEGKAYFPREGVFVF |
| | | NGTSWFITQRNFFSPQLITTDNTFVSGNCDVVIGIIN |
| | | NTVYDPLQPELDSFKEELDKYFKNHTSPDVDLGDISRINASVV |
| | | NIQKEIDRLNEVAKNLNESLIDLQELGKYEQYIKWPW |
| | | YVWLGFIAGLIAIVMATILLCCMTSCCSCLKGACSCGSCCKFDE |
| | | DDSEPVLKGVKLHYT |
| 36 | SARS-related | MKFFILLSLLSFTTAQEGCGILSNKSNPALTQYFSSRRGFYYFD |
| | coronavirus | DTFRSSVRVLTTGYFLPFNSNLTGYSSRNSVTGRLI |
| | Spike protein | QFDNPNIPFKDGLYFAATERSNVIRGWIFGSTLDNTTQSAVLF |
| | (Genbank: | NNGTHIVINVCNFYFCQDPMLAVANGSHFKSWVFLNA |
| | APO40579) | TNCTYNRVHGFEIDPSPNTGSFIHLREHVFRNVDGFLYVYHNY |
| | | ERVDVYDNFPQGFSVLKPIFKLPFGLNITQFKVIMTL |
| | | FSPTTSSFNADASVYFVGHLKPLTMLAEFDENGTITDAVDCSQ |
| | | DPLSELKCTTKSLTVEKGIYQTSNFRVSPSTEVVRFP |
| | | NITNLCPFGQVFNASNFPSVYAWERLRISDCVADYAVLYNSSS |
| | | SFSTFKCYGVSPTKLNDLCFSSVYADYFVVKGDDVRQ |
| | | IAPAQTGVIADYNYKLPDDFTGCVLAWNTNSVDSKSGNNFYY |
| | | RLFRHGKIKPYERDISNVLYNSAGGTCSSISQLGCYEP |
| | | LKSYGFTPTVGVGYQPYRVVVLSFELLNAPATVCGPKKSTELV |
| | | KNKCVNFNFNGLTGTGVLTSSTKKFQPFQQFGRDVSD |
| | | FTDSVRDPKTFEILDISPCSYGGVSVITPGTNTSKAVAVLYQDV |
| | | NCTDVPTMIHVEQVSSDWRVYAFNSYGNMFQTQAGC |
| | | LVGAIYENTTYECDIPIGAGICAKFGSDKIRMGQESIVAYTMSIG |
| | | EDQSIAYSNNIIAIPTNFSISVTTEVLPVSMTKTS |
| | | VDCNMYICGDSTECSNLLLQYGSFCTQLNRALSGIAVEQDRN |
| | | TRDVFAQTKSIYKTPNIKDFGGFNFSQILPDPKKLSYR |
| | | SFIEDLLYNKVTLSDPGFMKQYGDCLGGINARDLICAQKFNGL |
| | | TVLPPLLTDDMIAAYTAALISGTATAGYTFGAGAALQ |
| | | IPFAMQMAYRFNGIGVTQNVLYENQKQIANQFNNAISKIQDSLT |
| | | TTSAALGKLQDVINQNAVALNTLVKQLSSNFGAISS |
| | | VLNDILSRLDKVEAEVQIDRLITGRLQSLQTYVTQQLIRAAEIRA |
| | | SANLAATKMSECVLGQSKRVDFCGKGYHLMSFPQA |
| | | APHGVVFLHVTYVPSQQQNFTTAPAICHNGKAYFPREGVFVM |
| | | NGTHWFITQRNFYSPQVITTDNTFESGSCDVVIGIINN |
| | | TVYDPLQPELESFKQELDKYFKNHTSPDVDFGDISGINASVVDI |
| | | KKEIAHLNEIAKNLNESLIDLQELGKYEQYVKWPWY |
| | | VWLGFVAGIVAILMSVIMLCCMTNCCSCFKGMCSCGSCCKFD |
| | | EDHSEPVSGVKLHYT |
| 37 | SARS-like | MKVLIFLSLLSLVSAQDGCDRFSSKKTPLLTQFPSSKRGLYYF |
| | coronavirus | DDIFRSSTRVLTSGHFLPFNSNVTRYTTQHSAPGEWM |
| | BatCoV/BB9904/ | IYFDNPNITFGDGVYFGAAERSNALRGWIFGSTMDNTTQSAIL |
| | BGR/2008 | YNNGTHIVISVCNFNLCEQPMIKVFAGRSYRSLIYTN |
| | Spike | AANCTYQRVHAFNITTATGNGNLIHFREHVFRNVDGFLYVYHN |
| | glycoprotein | YELVNLSHTNPSFPAGFSVLKPIFKLPFGLNITFVRA |
| | (Genbank: | IMTVFSASTSQFEADYSAYFVGHLKPLTMLVDFDENGTITDAV |
| | ALJ94036) | DCSQDPLSELKCTTKSFTVEKGIYQTSNFRVTPTTEV |
| | | VRFPNITKLCPFDQVFNASSFPSVYAWERVRITDCVANYAVLY |
| | | NSSVSFSTFQCYGVSPTKLNDLCFSSVYADYFIVKGD |
| | | DVRQIAPSQTGVIADYNYKLPDDFTGCVIAWNTNALDSNKDFY |
| | | YRLFRHGKIKPYGRDLSNIPYSPSGTCSTINNLNCFA |
| | | PLKSYGFTQSSGISFQPYRVVVLSFELLNAPATVCGPKQSTEL |
| | | VKDKCVNFNFNGLTGTGVLTSSSKKFQPFQQFGRDVS |
| | | DFTDSVRDPKTLEILDIAPCSYGGVSVITPGTNASNSVAVLYQD |
| | | VNCTDVPTMLHAEQISHDWRVYTFSSDSNIYQTQAG |
| | | CLIGAAYDNSSYECDIPIGAGICAKYGISSNTRLRSNSQSIVAYT |
| | | MSLGEDQSIAYSNNTIAIPMNFSISVTTEVLPVSM |
| | | TKTSVDCNMYICGDSTECSNLLLQYGSFCTQLNRALAGIAVEQ |
| | | DRNTRDVFAQTKAMYKTPSLKDFGGFNFSQILPDPAK |
| | | PSSRSFIEDLLYNKVTLADPGFMKQYGDCLGGVNARDLICAQK |
| | | FNGLTVLPPLLTDEMIAAYTAALVSGTATAGFTFGAG |
| | | AALQIPFAMQMAYRFNGIGVTQNVLYENQKQIANQFNKAISQI |
| | | QDSLSTTTTALGKLQDVINQNAIALNTLVKQLSSNFG |
| | | AISSVLNDILSRLDKVEAEVQIDRLITGRLQSLQTYVTQQLIRAA |
| | | EIRASANIAATKMSECVLGQSKRVDFCGKGYHLMS |
| | | FPQAAPHGVVFLHVTYVPSQEQNFTTAPAICHGGKAHFPREG |
| | | VFVTNGTHWFITQRNFYSPQPITTDNTFVSGNCDVVIG |
| | | IVNNTVYDPLQPELESFKEELDKYFKNHTSQNVSLDGLNNINA |
| | | SVVDIKKEIEHLNEIAKSLNESLIDLQELGKYEQYIK |
| | | WPWYVWLGFIAGLIAIVMATIMLCCMTSCCSCLKGVCSCASC |
| | | CKFDEDHSEPVLTGVKLHYT |
| 38 | Bat | MKFLAFLCLLGFANAQDGKCGTLSNKSPSKLTQTPSSRRGFY |
| | coronavirus | YFDDIFRSSIRVLTTGHFLPFNTNLTWYLTLKSNGKQR |
| | BM48- | IYYDNPNINFGDGVYFGLTEKSNVFRGWIFGSTLDNTTQSAVL |
| | 31/BGR/2008 | FNNGTHIVIDVCNFNFCADPMFAVNSGQPYKTWIYTS |
| | Spike protein | AANCTYHRAHAFNISTNMNPGKFKHFREHLFKNVDGFLYVYH |
| | (Genbank: | NYEPIDLNSGFPSGFSVLKPILKLPFGLNITYVKAIMT |
| | YP_003858584) | LFSSTQSNFDADASAYFVGHLKPLTMLVDFDENGTIIDAIDCSQ |
| | | DPLSELKCTTKSFTVEKGIYQTSNFRVTPTTEVVRF |
| | | PNITQLCPFNEVFNITSFPSVYAWERMRITNCVADYSVLYNSS |
| | | ASFSTFQCYGVSPTKLNDLCFSSVYADYFVVKGDDVR |
| | | QIAPAQTGVIADYNYKLPDDFTGCVIAWNTNSLDSSNEFFYRR |
| | | FRHGKIKPYGRDLSNVLFNPSGGTCSAEGLNCYKPLA |
| | | SYGFTQSSGIGFQPYRVVVLSFELLNAPATVCGPKQSTELVKN |
| | | KCVNFNFNGLTGTGVLTNSTKKFQPFQQFGRDVSDFT |
| | | DSVRDPKTLEILDIAPCSYGGVSVITPGTNASSSVAVLYQDVNC |
| | | TDVPTMLHADQISHDWRVYAFRNDGNIFQTQAGCLI |
| | | GAAYDNSSYECDIPIGAGICAKYTNVSSTLVRSGGHSILAYTMS |
| | | LGDNQDIVYSNNTIAIPMNFSISVTTEVLPVSMTKT |
| | | SVDCNMYICGDSTECSNLLLQYGSFCTQLNRALAGIAVEQDR |
| | | NTRDVFAQTKAMYKTPSLKDFGGFNFSQILPDPAKPSS |
| | | RSFIEDLLYNKVTLADPGFMKQYGDCLGGVNARDLICAQKFN |
| | | GLTVLPPLLTDEMIAAYTAALISGTATAGFTFGAGAAL |
| | | QIPFAMQMAYRFNGIGVTQNVLYENQKQIANQFNKAISQIQDS |
| | | LSTTTTALGKLQDVINQNAIALNTLVKQLSSNFGAIS |
| | | SVLNDILSRLDKVEAEVQIDRLITGRLQSLQTYVTQQLIRAAEIR |
| | | ASANLAATKMSECVLGQSKRVDFCGKGYHLMSFPQ |
| | | AAPHGVVFLHVTYVPSQEQNFTTAPAICHEGKAHFPREGVFV |
| | | TNGTHWFITQRNFYSPQPITTDNTFVSGNCDVVIGIVN |
| | | NTVYDPLQPELDSFKEELDKYFKNHTSQNVSLDGLNNINASVV |
| | | DIKKEIEHLNEIAKSLNESLIDLQELGKYEQYIKWPW |
| | | YVWLGFIAGLIAIVMATIMLCCMTSCCSCLKGVCSCASCCKFD |
| | | EDHSEPVLTGVKLHYT |
| 39 | Pangolin | MLFFFFLHFALVNSQCVNLTGRAAIQPSFTNSSQRGVYYPDTI |
| | coronavirus | FRSNTLVLSQGYFLPFYSNVSWYYALTKTNSAEKRVD |
| | Surface | NPVLDFKDGIYFAATEKSNIVRGWIFGTTLDNTSQSLLIVNNAT |
| | glycoprotein | NVIIKVCNFQFCYDPYLSGYYHNNKTWSTREFAVYS |
| | (Genbank: | SYANCTFEYVSKSFMLDIAGKSGLFDTLREFVFRNVDGYFKIY |
| | QLR06867) | SKYTPVNVNSNLPIGFSALEPLVEIPAGINITKFRTL |
| | | LTIHRGDPMPNNGWTVFSAAYYVGYLAPRTFMLNYNENGTIT |
| | | DAVDCALDPLSEAKCTLKSLTVEKGIYQTSNFRVQPTE |
| | | SIVRFPNITNLCPFGEVFNATTFASVYAWNRKRISNCVADYSVL |
| | | YNSTSFSTFKCYGVSPTKLNDLCFTNVYADSFVVRG |
| | | DEVRQIAPGQTGRIADYNYKLPDDFTGCVIAWNSNNLDSKVG |
| | | GNYNYLYRLFRKSNLKPFERDISTEIYQAGSTPCNGVE |
| | | GFNCYFPLQSYGFHPTNGVGYQPYRVVVLSFELLNAPATVCG |
| | | PKQSTNLVKNKCVNFNFNGLTGTGVLTESSKKFLPFQQ |
| | | FGRDIADTTDAVRDPQTLEILDITPCSFGGVSVITPGTNTSNQV |
| | | AVLYQDVNCTEVPVAIHADQLTPTWSVYSTGSNVFQ |
| | | TRAGCLIGAEHVNNSYECDIPIGAGICASYQTQTNSRSVSSQAI |
| | | IAYTMSLGAENSVAYANNSIAIPTNFTISVTTEILP |
| | | VSMTKTSVDCTMYICGDSIECSNLLLQYGSFCTQLNRALTGIA |
| | | VEQDKNTQEVFAQVKQIYKTPPIKDFGGFNFSQILPD |
| | | PSKPSKRSFIEDLLFNKVTLADAGFIKQYGDCLGDIAARDLICA |
| | | QKFNGLTVLPPLLTDEMIAQYTSALLAGTITSGWTF |
| | | GAGAALQIPFAMQMAYRFNGIGVTQNVLYENQKLIANQFNSAI |
| | | GKIQDSLSSTASALGKLQDVVNQNAQALNTLVKQLSS |
| | | NFGAISSVLNDILSRLDKVEAEVQIDRLITGRLQSLQTYVTQQLI |
| | | RAAEIRASANLAATKMSECVLGQSKRVDFCGKGYH |
| | | LMSFPQSAPHGVVFLHVTYVPSQEKNFTTTPAICHEGKAHFPR |
| | | EGVFVSNGTHWFVTQRNFYEPQIITTDNTFVSGSCDV |
| | | VIGIVNNTVYDPLQPELDSFKEELDKYFKNHTSPDVDLGDISGI |
| | | NASVVNIQKEIDRLNEVAKNLNESLIDLQELGKYEQ |
| | | YIKWPWYIWLGFIAGLIAIIMVTIMLCCMTSCCSCLKGCCSCGS |
| | | CCKFDEDDSEPVLKGVKLHYT |
| 40 | Pangolin | MLFFFFLHFALVNSQCVNLTGRAAIQPSFTNSSQRGVYYPDTI |
| | coronavirus | FRSNTLVLSQGYFLPFYSNVSWYYALTKTNSAEKRVD |
| | Surface | NPVLDFKDGIYFAATEKSNIVRGWIFGTTLDNTSQSLLIVNNAT |
| | glycoprotein | NVIIKVCNFQFCYDPYLSGYYHNNKTWSTREFAVYS |
| | (Genbank: | SYANCTFEYVSKSFMLDIAGKSGLFDTLREFVFRNVDGYFKIY |
| | QLR06864) | SKYTPVNVNSNLPIGFSALEPLVEIPAGINITKFRTL |
| | | LTIHRGDPMPNNGWTVFSAAYYVGYLAPRTFMLNYNENGTIT |
| | | DAVDCALDPLSEAKCTLKSLTVEKGIYQTSNFRVQPTE |
| | | SIVRFPNITNLCPFGEVFNATTFASVYAWNRKRISNCVADYSVL |
| | | YNSTSFSTFKCYGVSPTKLNDLCFTNVYADSFVVRG |
| | | DEVRQIAPGQTGRIADYNYKLPDDFTGCVIAWNSNNLDSKVG |
| | | GNYNYLYRLFRKSNLKPFERDISTEIYQAGSTPCNGVE |
| | | GFNCYFPLQSYGFHPTNGVGYQPYRVVVLSFELLNAPATVCG |
| | | PKQSTNLVKNKCVNFNFNGLTGTGVLTESSKKFLPFQQ |
| | | FGRDIADTTDAVRDPQTLEILDITPCSFGGVSVITPGTNTSNQV |
| | | AVLYQDVNCTEVPVAIHADQLTPTWRVYSTGSNVFQ |
| | | TRAGCLIGAEHVNNTYECDIPIGAGICASYQTQTNSRSVSSQAI |
| | | IAYTMSLGAENSVAYANNSIAIPTNFTISVTTEILP |
| | | VSMTKTSVDCTMYICGDSIECSNLLLQYGSFCTQLNRALTGIA |
| | | VEQDKNTQEVFAQVKQIYKTPPIKDFGGFNFSQILPD |
| | | PSKPSKRSFIEDLLFNKVTLADAGFIKQYGDCLGDIAARDLICA |
| | | QKFNGLTVLPPLLTDEMIAQYTSALLAGTITSGWTF |
| | | GAGAALQIPFAMQMAYRFNGIGVTQNVLYENQKLIANQFNSAI |
| | | GKIQDSLSSTASALGKLQDVVNQNAQALNTLVKQLSS |
| | | NFGAISSVLNDILSRLDKVEAEVQIDRLITGRLQSLQTYVTQQLI |
| | | RAAEIRASANLAATKMSECVLGQSKRVDFCGKGYH |
| | | LMSFPQSAPHGVVFLHVTYVPSQEKNFTTTPAICHEGKAHFPR |
| | | EGVFVSNGTHWFVTQRNFYEPQIITTDNTFVSGSCDV |
| | | VIGIVNNTVYDPLQPELDSFKEELDKYFKNHTSPDVDLGDISGI |
| | | NASVVNIQKEIDRLNEVAKNLNESLIDLQELGKYEQ |
| | | YIKWPWYIWLGFIAGLIAIIMVTIMLCCMTSCCSCLKGCCSCGS |
| | | CCKFDEDDSEPVLKGVKLHYT |
| 41 | Pangolin | MLFFFFLHFALVNSQCVNLTGRAAIQPSFTNSSQRGVYYPDTI |
| | coronavirus | FRSNTLVLSQGYFLPFYSNVSWYYALTKTNSAEKRVD |
| | Surface | NPVLDFKDGIYFAATEKSNIVRGWIFGTTLDNTSQSLLIVNNAT |
| | glycoprotein | NVIIKVCNFQFCYDPYLSGYYHNNKTWSTREFAVYS |
| | (Genbank: | SYANCTFEYVSKSFMLDIAGKSGLFDTLREFVFRNVDGYFKIY |
| | QLR06866) | SKYTPVNVNSNLPIGFSALEPLVEIPAGINITKFRTL |
| | | LTIHRGDPMPNNGWTVFSAAYYVGYLAPRTFMLNYNENGTIT |
| | | DAVECALDPLSEAKCTLKSLTVEKGIYQTSNFRVQPTE |
| | | SIVRFPNITNLCPFGEVFNATTFASVYAWNRKRISNCVADYSVL |
| | | YNSTSFSTFKCYGVSPTKLNDLCFTNVYADSFVVRG |
| | | DEVRQIAPGQTGRIADYNYKLPDDFTGCVIAWNSNNLDSKVG |
| | | GNYNYLYRLFRKSNLKPFERDISTEIYQAGSTPQNGVE |
| | | GFNCYFPLQSYGFHPTNGVGYQPYRVVVLSFELLNAPATVCG |
| | | PKQSTNLVKNKCVNFNFNGLTGTGVLTESSKKFLPFQQ |
| | | FGRDIADTTDAVRDPQTLEILDITPCSFGGVSVITPGTNTSNQV |
| | | AVLYQDVNCTEVPVAIHADQLTPTWRVYSTGSNVFQ |
| | | TRAGCLIGAEHVNNTYECDIPIGAGICASYQTQTNSRSVSSQAI |
| | | IAYTMSLGAENSVAYANNSIAIPTNFTISVTTEILP |
| | | VSMTKTSVDCTMYICGDSIECSNLLLQYGSFCTQLNRALTGIA |
| | | VEQDKNTQEVFAQVKQIYKTPPIKDFGGFNFSQILPD |
| | | PSKPSKRSFIEDLLFNKVTLADAGFIKQYGDCLGDIAARDLICA |
| | | QKFNGLTVLPPLLTDEMIAQYTSALLAGTITSGWTF |
| | | GAGAALQIPFAMQMAYRFNGIGVTQNVLYENQKLIANQFNSAI |
| | | GKIQDSLSSTASALGKLQDVVNQNAQALNTLVKQLSS |
| | | NFGAISSVLNDILSRLDKVEAEVQIDRLITGRLQSLQTYVTQQLI |
| | | RAAEIRASANLAATKMSECVLGQSKRVDFCGKGYH |
| | | LMSFPQSAPHGVVFLHVTYVPSQEKNFTTTPAICHEGKAHFPR |
| | | EGVFVSNGTHWFVTQRNFYEPQIITTDNTFVSGSCDV |
| | | VIGIVNNTVYDPLQPELDSFKEELDKYFKNHTSPDVDLGDISGI |
| | | NASVVNIQKEIDRLNEVAKNLNESLIDLQELGKYEQ |
| | | YIKWPWYIWLGFIAGLIAIIMVTIMLCCMTSCCSCLKGCCSCGS |
| | | CCKFDEDDSEPVLKGVKLHYT |
| 42 | Bat | MFVFLVLLPLVSSQCVNLTTRTQLPPAYTNSSTRGVYYPDKVF |
| | coronavirus | RSSVLHLTQDLFLPFFSNVTWFHAIHVSGTNGIKRFD |
| | RaTG13 | NPVLPFNDGVYFASTEKSNIIRGWIFGTTLDSKTQSLLIVNNAT |
| | Spike | NVVIKVCEFQFCNDPFLGVYYHKNNKSWMESEFRVY |
| | glycoprotein | SSANNCTFEYVSQPFLMDLEGKQGNFKNLREFVFKNIDGYFKI |
| | (Genbank: | YSKHTPINLVRDLPPGFSALEPLVDLPIGINITRFQT |
| | QHR63300) | LLALHRSYLTPGDSSSGWTAGAAAYYVGYLQPRTFLLKYNEN |
| | | GTITDAVDCALDPLSETKCTLKSFTVEKGIYQTSNFRV |
| | | QPTDSIVRFPNITNLCPFGEVFNATTFASVYAWNRKRISNQVA |
| | | DYSVLYNSTSFSTFKCYGVSPTKLNDLCFTNVYADSF |
| | | VITGDEVRQIAPGQTGKIADYNYKLPDDFTGCVIAWNSKHIDAK |
| | | EGGNFNYLYRLFRKANLKPFERDISTEIYQAGSKPC |
| | | NGQTGLNCYYPLYRYGFYPTDGVGHQPYRVVVLSFELLNAPA |
| | | TVCGPKKSTNLVKNKCVNFNFNGLTGTGVLTESNKKFL |
| | | PFQQFGRDIADTTDAVRDPQTLEILDITPCSFGGVSVITPGTNA |
| | | SNQVAVLYQDVNCTEVPVAIHADQLTPTWRVYSTGS |
| | | NVFQTRAGCLIGAEHVNNSYECDIPIGAGICASYQTQTNSRSV |
| | | ASQSIIAYTMSLGAENSVAYSNNSIAIPTNFTISVTT |
| | | EILPVSMTKTSVDCTMYICGDSTECSNLLLQYGSFCTQLNRAL |
| | | TGIAVEQDKNTQEVFAQVKQIYKTPPIKDFGGFNFSQ |
| | | ILPDPSKPSKRSFIEDLLFNKVTLADAGFIKQYGDCLGDIAARDL |
| | | ICAQKFNGLTVLPPLLTDEMIAQYTSALLAGTITS |
| | | GWTFGAGAALQIPFAMQMAYRFNGIGVTQNVLYENQKLIANQ |
| | | FNSAIGKIQDSLSSTASALGKLQDVVNQNAQALNTLVK |
| | | QLSSNFGAISSVLNDILSRLDKVEAEVQIDRLITGRLQSLQTYVT |
| | | QQLIRAAEIRASANLAATKMSECVLGQSKRVDFCG |
| | | KGYHLMSFPQSAPHGVVFLHVTYVPAQEKNFTTAPAICHDGK |
| | | AHFPREGVFVSNGTHWFVTQRNFYEPQIITTDNTFVSG |
| | | SCDVVIGIVNNTVYDPLQPELDSFKEELDKYFKNHTSPDVDLG |
| | | DISGINASVVNIQKEIDRLNEVAKNLNESLIDLQELG |
| | | KYEQYIKWPWYIWLGFIAGLIAIIMVTIMLCCMTSCCSCLKGCC |
| | | SCGSCCKFDEDDSEPVLKGVKLHYT |
| 43 | SARS-CoV2 | MFVFLVLLPLVSSQCVNLTTRTQLPPAYTNSFTRGVYYPDKVF |
| | Surface | RSSVLHSTQDLFLPFFSNVTWFHAIHVSGTNGTKRFD |
| | glycoprotein | NPVLPFNDGVYFASTEKSNIIRGWIFGTTLDSKTQSLLIVNNAT |
| | (Genbank: | NVVIKVCEFQFCNDPFLGVYYHKNNKSWMESEFRVY |
| | YP_009724390) | SSANNCTFEYVSQPFLMDLEGKQGNFKNLREFVFKNIDGYFKI |
| | | YSKHTPINLVRDLPQGFSALEPLVDLPIGINITRFQT |
| | | LLALHRSYLTPGDSSSGWTAGAAAYYVGYLQPRTFLLKYNEN |
| | | GTITDAVDCALDPLSETKCTLKSFTVEKGIYQTSNFRV |
| | | QPTESIVRFPNITNLCPFGEVFNATRFASVYAWNRKRISNCVA |
| | | DYSVLYNSASFSTFKCYGVSPTKLNDLCFTNVYADSF |
| | | VIRGDEVRQIAPGQTGKIADYNYKLPDDFTGCVIAWNSNNLDS |
| | | KVGGNYNYLYRLFRKSNLKPFERDISTEIYQAGSTPC |
| | | NGVEGFNCYFPLQSYGFQPTNGVGYQPYRVVVLSFELLHAPA |
| | | TVCGPKKSTNLVKNKCVNFNFNGLTGTGVLTESNKKFL |
| | | PFQQFGRDIADTTDAVRDPQTLEILDITPCSFGGVSVITPGTNT |
| | | SNQVAVLYQDVNCTEVPVAIHADQLTPTWRVYSTGS |
| | | NVFQTRAGCLIGAEHVNNSYECDIPIGAGICASYQTQTNSPRR |
| | | ARSVASQSIIAYTMSLGAENSVAYSNNSIAIPTNFTI |
| | | SVTTEILPVSMTKTSVDCTMYICGDSTECSNLLLQYGSFCTQL |
| | | NRALTGIAVEQDKNTQEVFAQVKQIYKTPPIKDFGGF |
| | | NFSQILPDPSKPSKRSFIEDLLFNKVTLADAGFIKQYGDCLGDI |
| | | AARDLICAQKFNGLTVLPPLLTDEMIAQYTSALLAG |
| | | TITSGWTFGAGAALQIPFAMQMAYRFNGIGVTQNVLYENQKLI |
| | | ANQFNSAIGKIQDSLSSTASALGKLQDVVNQNAQALN |
| | | TLVKQLSSNFGAISSVLNDILSRLDKVEAEVQIDRLITGRLQSLQ |
| | | TYVTQQLIRAAEIRASANLAATKMSECVLGQSKRV |
| | | DFCGKGYHLMSFPQSAPHGVVFLHVTYVPAQEKNFTTAPAIC |
| | | HDGKAHFPREGVFVSNGTHWFVTQRNFYEPQIITTDNT |
| | | FVSGNCDVVIGIVNNTVYDPLQPELDSFKEELDKYFKNHTSPD |
| | | VDLGDISGINASWVNIQKEIDRLNEVAKNLNESLIDL |
| | | QELGKYEQYIKWPWYIWLGFIAGLIAIVMVTIMLCCMTSCCSCL |
| | | KGCCSCGSCCKFDEDDSEPVLKGVKLHYT |
| 44 | SARS-CoV2 | MFVFFVLLPLVFIQCVSFTNIIHLPSSYINSFTRGVYYPDKVFRS |
| | VOC_May_01 | SVLYSIRDFFLPFFSNVTWFHVIRTNVIKRFANPV |
| | | LPFNDGVYFAFIDKFNIIRGWIFGTTLDSKTQSLLIVNNATNVVIK |
| | | VCEFQFCNYPFYKNNKSCTKSESRVYSSATNCTF |
| | | EYVSKPFFIDLVVKQGNFKNFSEFVFKNIDGYFKIYSKHTPNLV |
| | | RGFPQGFLVLEPLVDLPIGINITRFQILYRSYLTPG |
| | | GFFLGLTAGSAAYYVGYLQLRTFLLKYNENGTITDAVDCALDP |
| | | LSEIKCTLKSFILEKGIYQTSNFRVQPTESIVRFPNI |
| | | TNLCPFGEVFNATRFASVYAWNRKRISNCVADYSFLYNSASF |
| | | STFKCYGVSLTKLNDLCFTNVYADSFVIRGDEVRQIAP |
| | | GKTGNIADYNYKLPDDFTGCVIAWNSKKLDSKVGGNYKYRFR |
| | | LFRKSNLKPFERDISTEIYQAGNKPCNGVKGFNCYSPL |
| | | QPYGFQPTYGVGYQPYRVVVLSFELLHSPSTVCGPKKSTNLV |
| | | KNKCVNFNFNGLIGTGVLTESNKNFLPFQQFGRDIDDI |
| | | TDAVRDPQTLDILDITPCSFGGVSVITPGTNTSNQVAVLYHGV |
| | | NCTEVPFAIHSDQLTPTWRVYSTGFNVFQTRAGCLIG |
| | | VEYVNNSYECDIPIGAGICATYHTHTKSHRRARSVVSQSIIAYT |
| | | MSLGVENLVVYSNNSIAIPINFTISVITEILPVSMA |
| | | KTSVDCTMYICGDSTECSNLLLQYGSFCTQLNRALTVIAIEQDK |
| | | NTQQVFAQVKQIYKIPPIKYFGGFNFSQILPDSSKS |
| | | SKRSFIEDLFFNKVTLADAGFIKQYGDCLGDISARDLICAQKFN |
| | | GLNVLPPLLTDEMIAQYTSALLSGTITSGWTLGAGA |
| | | ALQIPFSMQMAYRFNGIGVTQNVLYENQKLIANQFNSAIGKIQY |
| | | SFFFTAPALGKLQHVVNQNARALNTLVKQLSSNFGA |
| | | ISSVLNDILARLDKVEAEVQIDRLITGRLQSLQTYVTQQLIRAAEI |
| | | RSSANLAAIKMSECVLGQSKRVDFCGKGYHLMSF |
| | | PQSAPHGVVFFHVTYVPALENNFTTSPAICHYGKAHFPREGVF |
| | | VSNGTYWFLTQRNFYEPQIITIHNTFLSGNCDVVIGI |
| | | VNNTVYDPLQPELDSFKEELDKYFKNHTSSYVDLVDISGINASF |
| | | VNIQKEIDRLNEVANNLNESLIDLQQLGKYEQYIKW |
| | | PWYIWLCFIAGLIAILIVTIMLCCITSCCSCLKGCCSCGFCCKFD |
| | | EDDSELLLKGVKLHYT |
| 45 | SARS-CoV2 | MFVFFVLLPLVFIQCVNFTIIIHLPSSYINSFTRGVYYPDKVFRSS |
| | Voc | VLYSIRDFFLPFFSNVTWFHVIRTNVIKRFANPV |
| | Whole VOC | LPFNDGVYFASIDKFNIIRGWIFGTTLDSKTQSLLIVNNATNVVI |
| | surface | KVCEFQFCNYPFYKNNKSCTESESRVYSSATNCTF |
| | glycoprotein | EYVSKPFFIDLVVKQGNFKNFSEFVFKNIDGYFKIYSKHTPNLV |
| | (Genbank: | RGFPQGFLVLEPLVDLPIGINITRFQTLYRSYLTPG |
| | YP_009724390) | GFFLGLTAGSAAYYVGYLQLRTFLLKYNENGTITDAVDCALDP |
| | | LSEIKCTLKSFILEKGIYQTSNFRVQPTESIVRFPNI |
| | | TNLCPFGEVFNATRFASVYAWNRKRISNCVADYSFLYNSASF |
| | | STFKCYGVSLTKLNDLCFTNVYADSFVIRGDEVRQIAP |
| | | GKTGNIADYNYKLPDDFTGCVIAWNSKKLDSKVGGNYNYRFR |
| | | LFRKSNLKPFERDISTEIYQAGNKPCNGVKGFNCYSPL |
| | | QPYGFQPTYGVGYQPYRVVVLSFELLHSPSTVCGPKKSTNLV |
| | | KNKCVNFNFNGLIGTGVLTESNKNFLPFQQFGRDIDDI |
| | | TDAVRDPQTLDILDITPCSFGGVSVITPGTNTSNQVAVLYHGV |
| | | NCTEVPFAIHSDQLTPTWRVYSTGFNVFQTRAGCLIG |
| | | VQYVNNSYECDIPIGAGICATYHTHTKSHRRARSVVSQSIIAYT |
| | | MSLGVENLVVYSNNSIAIPINFTISVITEILPVSMA |
| | | KTSVDCTMYICGDSTECSNLLLQYGSFCTQLNRALTVIAIEQDK |
| | | NTQQVFAQVKQIYKIPPIKYFGGFNFSQILPDSSKS |
| | | SKRSFIEDLFFNKVTLADAGFIKQYGDCLGDISARDLICAQKFN |
| | | GLNVLPPLLTDEMIAQYTSALLSGTITSGWTLGAGA |
| | | ALQIPFSMQMAYRFNGIGVTQNVLYENQKLIANQFNSAIGKIQY |
| | | SFFFTASALGKLQHVVNQNARALNTLVKQLSSNFGA |
| | | ISSVLNDILARLDKVEAEVQIDRLITGRLQSLQTYVTQQLIRAAEI |
| | | RSSANLAAIKMSECVLGQSKRVDFCGKGYHLMSF |
| | | PQSAPHGVVFFHVTYVPALENNFTTSPAICHYGKAHFPREGVF |
| | | VSNGTHWFLTQRNFYEPQIITIHNTFLSGNCDVVIGI |
| | | VNNTVYDPLQPELDSFKEELDKYFKNHTSSYVDLVDISGINASF |
| | | VNIQKEIDRLNEVANNLNESLIDLQQLGKYEQYIKW |
| | | PWYIWLCFIAGFIAILIVTIMLCCITSCCSCLKGCCSCGFCCKFD |
| | | EDDSELLLKGVKLHYT |
| 46 | Pangolin | MFVFLFVLPLVSSQCVNLTTRTGIPPGYTNSSTRGVYYPDKVF |
| | coronavirus | RSSILHLTQDLFLPFFSNVTWFNTINYQGGFKKFDNP |
| | Spike protein | VLPFNDGVYFASTEKSNIIRGWIFGTTLDARTQSLLIVNNATNV |
| | (Genbank: | VIKVCEFQFCTDPFLGVYYHNNNKTWVENEFRVYSS |
| | QIA48614) | ANNCTFEYISQPFLMDLEGKQGNFKNLREFVFKNVDGYFKIYS |
| | | KHTPIDLVRDLPRGFAALEPLVDLPIGINITRFQTLL |
| | | ALHRSYLTPGNLESGWTTGAAAYYVGYLQQRTFLLSYNQNGT |
| | | ITDAVDCSLDPLSETKCTLKSLTVEKGIYQTSNFRVQP |
| | | TISIVRFPNITNLCPFGEVFNASKFASVYAWNRKRISNCVADYS |
| | | VLYNSTSFSTFKCYGVSPTKLNDLCFTNVYADSFVV |
| | | KGDEVRQIAPGQTGVIADYNYKLPDDFTGCVIAWNSVKQDALT |
| | | GGNYGYLYRLFRKSKLKPFERDISTEIYQAGSTPQNG |
| | | QVGLNCYYPLERYGFHPTTGVNYQPFRVVVLSFELLNGPATV |
| | | CGPKLSTTLVKDKCVNFNFNGLTGTGVLTTSKKQFLPF |
| | | QQFGRDISDTTDAVRDPQTLEILDITPCSFGGVSVITPGTNTSN |
| | | QVAVLYQDVNCTEVPMAIHAEQLTPAWRVYSAGANV |
| | | FQTRAGCLVGAEHVNNSYECDIPVGAGICASYHSMSSLRSVN |
| | | QRSIIAYTMSLGAENSVAYSNNSIAIPTNFTISVTTEI |
| | | LPVSMTKTSVDCTMYICGDSIECSNLLLQYGSFCTQLNRALTGI |
| | | AVEQDKNTQEVFAQVKQIYKTPPIKDFGGFNFSQIL |
| | | PDPSKPSKRSFIEDLLFNKVTLADAGFIKQYGDCLGDIAARDLI |
| | | CAQKFNGLTVLPPLLTDEMIAQYTSALLAGTITSGW |
| | | TFGAGAALQIPFAMQMAYRFNGIGVTQNVLYENQKLIANQFNS |
| | | AIGKIQDSLSSTASALGKLQDVVNQNAQALNTLVKQL |
| | | SSNFGAISSVLNDILSRLDKVEAEVQIDRLITGRLQSLQTYVTQ |
| | | QLIRAAEIRASANLAATKMSECVLGQSKRVDFCGKG |
| | | YHLMSFPQSAPHGVVFLHVTYVPAQEKNFTTAPAICHEGKAH |
| | | FPREGVFVSNGTHWFITQRNFYEPQIITTDNTFVSGSC |
| | | DVVIGIVNNTVYDPLQPELDSFKEELDKYFKNHTSPDVDLGDIS |
| | | GINASVVNIQKEIDRLNEVAKNLNESPIDLQELGKY |
| | | EQYIKWPWYIWLGFIAGLIAIIMVTIMLCCMTSCCSCLKGCCSC |
| | | GSCCKFDEDDSEPVLKGVKLHYT |
| 47 | Pangolin | MFVFLFVLPLVSSQCVNLTTRTGIQPGYTNSSTRGVYYPDKVF |
| | coronavirus | RSSILHLTQDLFLPFFSNVTWFNTINYQGGFKKFDNP |
| | Spike protein | VLPFNDGVYFASTEKSNIIRGWIFGTTLDARTQSLLIVNNATNV |
| | (Genbank: | VIKVCEFQFCTDPFLGVYYHNNNKTWVENEFRVYSS |
| | QIA48623) | ANNCTFEYISQPFLMDLEGKQGNFKNLREFVFKNVDGYFKIYS |
| | | KHTPIDLVRDLPRGFAALEPLVDLPIGINITRFQTLL |
| | | ALHRSYLTPGKLESGWTTGAAAYYVGYLQQRTFLLSYNQNGT |
| | | ITDAVDCSLDPLSETKCTLKSLTVEKGIYQTSNFRVQP |
| | | TISIVRFPNITNLCPFGEVFNASKFASVYAWNRKRISNCVADYS |
| | | VLYNSTSFSTFKCYGVSPTKLNDLCFTNVYADSFVV |
| | | KGDEVRQIAPGQTGVIADYNYKLPDDFTGCVIAWNSVKQDALT |
| | | GGNYLYRLFRKSKLKPFERDISTEIYQAGSTPCNGQV |
| | | GLNCYYPLERYGFHPTTGVNYQPFRVVVLSFELLNGPATVCG |
| | | PKLSTTLVKDKCVNFNFNGLTGTGVLTTSKKQFLPFQQ |
| | | FGRDISDTTDAVRDPQTLEILDITPCSFGGVSVITPGTNTSNQV |
| | | AVLYQDVNCTEVPMAIHAEQLTPAWRVYSAGANVFQ |
| | | TRAGCLVGAEHVNNSYECDIPVGAGICASYHSMSSLRSVNQR |
| | | SIIAYTMSLGAENSVAYSNNSIAIPTNFTISVTTEILP |
| | | VSMTKTSVDCTMYICGDSIECSNLLLQYGSFCTQLNRALTGIA |
| | | VEQDKNTQEVFAQVKQIYKTPPIKDFGGFNFSQILPD |
| | | PSKPSKRSFIEDLLFNKVTLADAGFIKQYGDCLGDIAARDLICA |
| | | QKFNGLTVLPPLLTDEMIAQYTSALLAGTITSGWTF |
| | | GAGAALQIPFAMQMAYRFNGIGVTQNVLYENQKLIANQFNSAI |
| | | GKIQDSLSSTASALGKLQDVVNQNAQALNTLVKQLSS |
| | | NFGAISSVLNDILSRLDKVEAEVQIDRLITGRLQSLQTYVTQQLI |
| | | RAAEIRASANLAATKMSECVLGQSKRVDFCGKGYH |
| | | LMSFPQSAPHGVVFLHVTYVPAQEKNFTTAPAICHEGKAHFP |
| | | REGVFVSNGTHWFITQRNFYEPQIITTDNTFVSGSCDV |
| | | VIGIVNNTVYDPLQPELDSFKEELDKYFKNHTSPDVDLGDISGI |
| | | NASVVNIQKEIDRLNEVAKNLNESLIDLQELGKYEQ |
| | | YIKWPWYIWLGFIAGLIAIIMVTIMLCCMTSCCSCLKGCCSCGS |
| | | CCKFDEDDSEPVLKGVKLHYT |
| 48 | Pangolin | MFVFLFVLPLVSSQCVNLTTRTGIPPGYTNSSTRGVYYPDKVF |
| | coronavirus | RSSILHLTQDLFLPFFSNVTWFNTITYQGGSKKFDNP |
| | Spike protein | VLPFNDGVYFASTEKSNIIRGWIFGTTLDARTQSLLIVNNATNV |
| | (Genbank: | VIKVCEFQFCTDPFLGVYYHNNNKTWVENEFRVYSS |
| | QIA48641) | ANNCTFEYISQPFLMDLEGKQGNFKNLREFVFKNVDGYFKIYS |
| | | KHTPIDLVRDLPRGFAALEPLVDLPIGINITRFQTLL |
| | | ALHRSYLTPGKLESGWTTGAAAYYVGYLQQRTFLLSYNQNGT |
| | | ITDAVDCSLDPLSETKCTLKSLTVEKGIYQTSNFRVQP |
| | | TISIVRFPNITNLCPFGEVFNASKFASVYAWNRKRISNCVADYS |
| | | VLYNSTSFSTFKCYGVSPTKLNDLCFTNVYADSFVV |
| | | KGDEVRQIAPGQTGVIADYNYKLPDDFTGCVIAWNSVKQDALT |
| | | GDNYGYLYRLFRKSKLKPFERDISTEIYQAGSTPCNG |
| | | QVGLNCYYPLERYGFHPTTGVNYQPFRVVVLSFELLNGPATV |
| | | CGPKLSTTLVKDKCVNFNFNGLTGTGVLTTSKKQFLPF |
| | | QQFGRDISDTTDAVRDPQTLEILDITPCSFGGVSVITPGTNTSN |
| | | QVAVLYQDVNCTEVPMAIHAEQLTPAWRVYSAGANV |
| | | FQTRAGCLVGAEHVNNSYECDIPVGAGICASYHSMSSFRSVN |
| | | QRSIIAYTMSLGAENSVAYSNNSIAIPTNFTISVTTEI |
| | | LPVSMTKTSVDCTMYICGDSIECSNLLLQYGSFCTQLNRALTGI |
| | | AVEQDKNTQEVFAQVKQIYKTPPIKDFGGFNFSQIL |
| | | PDPSKPSKRSFIEDLLFNKVTLADAGFIKQYGDCLGDIAARDLI |
| | | CAQKFNGLTVLPPLLTDEMIAQYTSALLAGTITSGW |
| | | TFGAGAALQIPFAMQMAYRFNGIGVTQNVLYENQKLIANQFNS |
| | | AIGKIQDSLSSTASALGKLQDVVNQNAQALNTLVKQL |
| | | SSNFGAISSVLNDILSRLDKVEAEVQIDRLITGRLQSLQTYVTQ |
| | | QLIRAAEIRASANLAATKMSECVLGQSKRVDFCGKG |
| | | YHLMSFPQSAPHGVVFLHVTYVPAQEKNFTTAPAICHEGKAH |
| | | FPREGVFVSNGTHWFITQRNFYEPQIITTDNTFVSGSC |
| | | DVVIGIVNNTVYDPLQPELDSFKEELDKYFKNHTSPDVDLGDIS |
| | | GINASVVNIQKEIDRLNEVAKNLNESLIDLQELGKY |
| | | EQYIKWPWYIWLGFIAGLIAIIMVTIMLCCMTSCCSCLKGCCSC |
| | | GSCCKFDEDDSEPVLKGVKLHYT |
| 49 | Pangolin | MFVFLFVLPLVSSQCVNLTTRTGIPPGYTNSSTRGVYYPDKVF |
| | coronavirus | RSSILHLTQDLFLPFFSNVTWFNTINYQGGFKKFDNP |
| | Spike protein | VLPFNDGVYFASTEKSNIIRGWIFGTTLDARTQSLLIVNNATNV |
| | (Genbank: | VIKVCEFQFCTDPFLGVYYHNNNKTWVENEFRVYSS |
| | QIA48632) | ANNCTFEYISQPFLMDLEGKQGNFKNLREFVFKNVDGYFKIYS |
| | | KHTPIDLVRDLPRGFAALEPLVDLPIGINITRFQTLL |
| | | ALHRSYLTPGKLESGWTTGAAAYYVGYLQQRTFLLSYNQNGT |
| | | ITDAVDCSLDPLSETKCTLKSLTVEKGIYQTSNFRVQP |
| | | TISIVRFPNITNLCPFGEVFNASKFASVYAWNRKRISNCVADYS |
| | | VLYNSTSFSTFKCYGVSPTKLNDLCFTNVYADSFVV |
| | | KGDEVRQIAPGQTGVIADYNYKLPDDFTGCVIAWNSVKQDALT |
| | | GGNYGYLYRLFRKSKLKPFERDISTEIYQAGSTPQNG |
| | | QVGLNCYYPLERYGFHPTTGVNYQPFRVVVLSFELLNGPATV |
| | | CGPKLSTTLVKDKCVNFNFNGLTGTGVLTTSKKQFLPF |
| | | QQFGRDISDTTDAVRDPQTLEILDITPCSFGGVSVITPGTNTSN |
| | | QVAVLYQDVNCTEVPMAIHAEQLTPAWRVYSAGANV |
| | | FQTRAGCLVGAEHVNNSYECDIPVGAGICASYHSMSSFRSVN |
| | | QRSIIAYTMSLGAENSVAYSNNSIAIPTNFTISVTTEI |
| | | LPVSMTKTSVDCTMYICGDSIECSNLLLQYGSFCTQLNRALTGI |
| | | AVEQDKNTQEVFAQVKQIYKTPPIKDFGGFNFSQIL |
| | | PDPSKPSKRSFIEDLLFNKVTLADAGFIKQYGDCLGDIAARDLI |
| | | CAQKFNGLTVLPPLLTDEMIAQYTSALLAGTITSGW |
| | | TFGAGAALQIPFAMQMAYRFNGIGVTQNVLYENQKLIANQFNS |
| | | AIGKIQDSLSSTASALGKLQDVVNQNAQALNTLVKQL |
| | | SSNFGAISSVLNDILSRLDKVEAEVQIDRLITGRLQSLQTYVTQ |
| | | QLIRAAEIRASANLAATKMSECVLGQSKRVDFCGKG |
| | | YHLMSFPQSAPHGVVFLHVTYVPAQEKNFTTAPAICHEGKAH |
| | | FPREGVFVSNGTHWFITQRNFYEPQIITTDNTFVSGSC |
| | | DVVIGIVNNTVYDPLQPELDSFKEELDKYFKNHTSPDVDLGDIS |
| | | GINASVVNIQKEIDRLNEVAKNLNESLIDLQELGKY |
| | | EQYIKWPWYIWLGFIAGLIAIIMVTIMLCCMTSCCSCLKGCCSC |
| | | GSCCKFDEDDSEPVLKGVKLHYT |
| 50 | Pangolin | MFVFLFVLPLVSSQCVNLTTRTGIPPGYTNSSTRGVYYPDKVF |
| | coronavirus | RSSILHLTQDLFLPFFSNVTWENTIHLNYQGGFKKFD |
| | Spike protein | NPVLPXNDGVYFASTEKSNIIRGWIFGTTLDARTQSLLIVNNAT |
| | (Genbank: | NVVIKVCEFQFCTDPFLGVYYHNNNKTWVENEFRVY |
| | QIQ54048) | SSANNCTFEYISQPFLMDLEGKQGNFKNLREFVFKNVDGYFKI |
| | | YSKHTPIDLVRDLPRGFAALEPLVDLPIGINITRFQT |
| | | LLALHRSYLTPGKLESGWTTGAAAYYVGYLQQRTFLLSYNQN |
| | | GTITDAVDCSLDPLSETKCTLKSLTVEKGIYQTSNFRV |
| | | QPTISIVRFPNITNLCPFGEVFNASKFASVYAWNRKRISNCVAD |
| | | YSVLYNSTSFSTFKCYGVSPTKLNDLCFTNVYADSF |
| | | VVKGDEVRQIAPGQTGVIADYNYKLPDDFTGCVIAWNSVKQD |
| | | ALTGGNYGYLYRLFRKSKLKPFERDISTEIYQAGSTPC |
| | | NGQVGLNCYYPLERYGFHPTTGVNYQPFRVVVLSXELLNGPA |
| | | TVCGPKLSTTLVKDKCVNFNFNGLTGTGVLTTSKKQFL |
| | | PFQQFGRDISDTTDAVRDPQTLEILDITPCSFGGVSVITPGTNT |
| | | SNQVAVLYQDVNCTEVPMAIHAEQLTPAWRVYSAGA |
| | | NVFQTRAGCLVGAEHVNNSYECDIPVGAGICASYHSMSSFRS |
| | | VNQRSIIAYTMSLGAENSVAYSNNSIAIPTNFTISVTT |
| | | EILPVSMTKTSVDCTMYICGDSIECSNLLLQYGSFCTQLNRALT |
| | | GIAVEQDKNTQEVFAQVKQIYKTPPIKDFGGFNFLQ |
| | | ILPDPSKPSKRSFIEDLLFNKVTLADAGFIKQYGDCLGDIAARDL |
| | | ICAQKFNGLTVLPPLLTDEMIAQYTSALLAGTITS |
| | | GWTFGAGAALQIPFAMQMAYRFNGIGVTQNVLYENQKLIANQ |
| | | FNSAIGKIQDSLSSTASALGKLQDVVNQNAQALNTLVK |
| | | QLSSNFGAISSVLNDILSRLDKVEAEVQIDRLITGRLQSLQTYVT |
| | | QQLIRAAEIRASANLAATKMSECVLGQSKRVDFCG |
| | | KGYHLMSFPQSAPHGVVFLHVTYVPAQEKNFTTAPAICHEGK |
| | | AHFPREGVFVSNGTHWFITQRNFYEPQIITTDNTFVSG |
| | | SCDVVIGIVNNTVYDPLQPELDSFKEELDKYFKNHTSPDVDLG |
| | | DISGINASVVNIQKEIDRLNEVAKNLNESLIDLQELG |
| | | KYEQYIKWPWYIWLGFIAGLIAIIMVTIMLCCMTSCCSCLKGCC |
| | | SCGSCCKFDEDDSEPVLKGVKLHYT |
| 51 | SC2r-CoV | MFILVLLNVLSVYGCVNITYGSHHLYVSSRTRGVYYPDDAFRS |
| | batACE2 | STNVLHEGFFLPFDSNVTWYSFWNQKYSVATSPFGDG |
| | Spike | VYFSTIDKSNVVRGWVFGTTLDNDTQSVLLYNDGTHVRVEVC |
| | | TFHFCPTPVFSASSPHLYSSAFNCTLNYTLASVRADFT |
| | | EVDGSFKTIREFVFKLQDGSLNVYYASTSYVLAIGATSQLPSG |
| | | VTPLVPLWKIPIGLNITNFKTLVYLRSDNTPLQAAYV |
| | | VGHLKRRTMMFKYDENGTIVDAIDCALDPLSETKCTLRSFIVEK |
| | | GIYQTSNFRVQPQDTVVRFPNITNLCPFSEVFNATT |
| | | FASVYAWNRKRISNCVADYSVLYNSTSFSTFQCYGVSSTKLN |
| | | DLCFTNVYADSFVVRGDEVRQIAPGQTGVIADYNYKLP |
| | | DDFTGCVLAWNSRNQDASTSGNFNYYYRIWRSEKLRPFERDI |
| | | AHYDYQVGTQFKSSLKNYGFYSSAGDSHQPYRVVVLSF |
| | | ELLNAPATVCGPKQSTELIKNKCVNFNFNGLTGTGVLTDSNKK |
| | | FQSFQQFGRDVSDFTDSVKDPKTLEVLDITPCSYGGV |
| | | SVITPGTNASTQVAVLYQDVNCTDVPTAIHAEQLTPSWRVYST |
| | | GTNMFQTQAGCLIGAEHVNNSYDCDIPIGAGICATYH |
| | | TPSMLRSANNNKRIVAYVMSLGAENSVAYSNNTIAIPTNFTISV |
| | | TTEVMPVSMTKTSVDCTMYICGDSVECSTLLLQYGS |
| | | FCTQLNRALTGIAVEQDKNTQEVFAQVKQIYKTPDIKDFGGFN |
| | | FSQILPDPSKPSKRSFIEDLLFNKVTLADAGFVKQYG |
| | | DCLGDIQARDLICAQKFNGLTVLPPLLTDEMIAAYTAALISGTAT |
| | | AGWTFGAGAALQIPFAMQMAYRFNGIGVTQNVLYE |
| | | NQKLIANQFNSAIGKIQESLTSTASALGKLQDVVNQNAQALNTL |
| | | VKQLSSNFGAISSVLNDIISRLDKVEAEVQIDRLIT |
| | | GRLQSLQTYVTQQLIRAAEIRASANLAATKMSECVLGQSKRVD |
| | | FCGKGYHLMSFPQAAPHGVVFLHVTYIPSQERNFTTA |
| | | PAICHEGKAHFPREGVFVSNGTHWFITQRNFYEPQIITTDNTFV |
| | | SGTCDVVIGIVNNTVYDPLQPELESFKDELDKYFKN |
| | | HTSPDIDLGDISGINASVVDIQKEIDILKDVAKNLNESLINLQELG |
| | | KYEQYIKWPWYVWLGFIAGLIAIIMVTIMLCCMT |
| | | SCCSCLKGCCSCGSCCKFDEDDSEPVLKGVKLHYT |
| 52 | Bats SARS | MFILVLLNVLSVYGCVNITYGSHHLYVSSRTRGVYYPDDAFRS |
| | coronavirus | STNVLHEGFFLPFDSNVTWYSFWNQKYSVATSPFGDG |
| | Rc-0319 | VYFSTIDKSNVVRGWVFGTTLDNDTQSVLLYNDGTHVRVEVC |
| | Spike | TFHFCPTPVFSASSPHLYSSAFNCTLNYTLASVRADFT |
| | (Genbank: | EVDGSFKTIREFVFKLQDGSLNVYYASTSYVLAIGATSQLPSG |
| | QIQ54048) | VTPLVPLWKIPIGLNITNFKTLVYLRSDNTPLQAAYV |
| | | VGHLKRRTMMFKYDENGTIVDAIDCALDPLSETKCTLRSFIVEK |
| | | GIYQTSNFRVQPQDTVVRFPNITNLCPFSEVENATT |
| | | FASVYAWNRKRISNCVADYSVLYNSTSFSTFQCYGVSSTKLN |
| | | DLCFTNVYADSFVVRGDEVRQIAPGQTGVIADYNYKLP |
| | | DDFTGCVLAWNSRNQDASTSGNFNYYYRIWRSEKLRPFERDI |
| | | AHYDYQVGTQFKSSLKNYGFYSSAGDSHQPYRVVVLSF |
| | | ELLNAPATVCGPKQSTELIKNKCVNFNFNGLTGTGVLTDSNKK |
| | | FQSFQQFGRDVSDFTDSVKDPKTLEVLDITPCSYGGV |
| | | SVITPGTNASTQVAVLYQDVNCTDVPTAIHAEQLTPSWRVYST |
| | | GTNMFQTQAGCLIGAEHVNNSYDCDIPIGAGICATYH |
| | | TPSMLRSANNNKRIVAYVMSLGAENSVAYSNNTIAIPTNFTISV |
| | | TTEVMPVSMTKTSVDCTMYICGDSVECSTLLLQYGS |
| | | FCTQLNRALTGIAVEQDKNTQEVFAQVKQIYKTPDIKDFGGFN |
| | | FSQILPDPSKPSKRSFIEDLLFNKVTLADAGFVKQYG |
| | | DCLGDIQARDLICAQKFNGLTVLPPLLTDEMIAAYTAALISGTAT |
| | | AGWTFGAGAALQIPFAMQMAYRFNGIGVTQNVLYE |
| | | NQKLIANQFNSAIGKIQESLTSTASALGKLQDVVNQNAQALNTL |
| | | VKQLSSNFGAISSVLNDIISRLDKVEAEVQIDRLIT |
| | | GRLQSLQTYVTQQLIRAAEIRASANLAATKMSECVLGQSKRVD |
| | | FCGKGYHLMSFPQAAPHGVVFLHVTYIPSQERNFTTA |
| | | PAICHEGKAHFPREGVFVSNGTHWFITQRNFYEPQIITTDNTFV |
| | | SGTCDVVIGIVNNTVYDPLQPELESFKDELDKYFKN |
| | | HTSPDIDLGDISGINASVVDIQKEIDILKDVAKNLNESLINLQELG |
| | | KYEQYIKWPWYVWLGFIAGLIAIIMVTIMLCCMT |
| | | SCCSCLKGCCSCGSCCKFDEDDSEPVLKGVKLHYT |
| 53 | Bat SARS-like | SNFRVAPSKEVVRFPNITNLCPFGEVFNATTFPSVYAWERKRI |
| | coronavirus | SNCVADYSVLYNSTSFSTFKCYGVSATKLNDLCFSNV |
| | Rs7327 | YADSFVVKGDDVRQIAPGQTGVIADYNYKLPDDFMGCVLAWN |
| | S protein | TRNIDATSTGNYNYKYRSLRHGKLRPFERDISNVPFSP |
| | RBD | DGKPCTPPAFNCYWPLNDYGFFTTNGIGYQPYRVVVLSFELL |
| | (Genbank: | NAPATVCGPKLSTDLIKNQCVNFNFNGLTGTGVLTSSS |
| | ATO98218) | KRFQPFQQFGRDVSDFTDSVRDPKTSEIL |
| 54 | Coronavirus | SNFRVAPSKEVVRFPNITNLCPFGEVFNATTFPSVYAWERKRI |
| | BtRs- | SNCVADYSVLYNSTSFSTFKCYGVSATKLNDLCFSNV |
| | BetaCoV/YN2018B | YADSFVVKGDDVRQIAPGQTGVIADYNYKLPDDFMGCVLAWN |
| | S protein | TRNIDATSTGNYNYKYRSLRHGKLRPFERDISNVPFSP |
| | RBD | DGKPCTPPAFNCYWPLNDYGFFTTNGIGYQPYRVVVLSFELL |
| | (Genbank: | NAPATVCGPKLSTDLIKNQCVNFNFNGLTGTGVLTPSS |
| | QDF43825) | KRFQPFQQFGRDVSDFTDSVRDPKTSEIL |
| 55 | Rhinolophus | SNFRVSPSKEVVRFPNITNLCPFGEVFNATTFPSVYAWERKRI |
| | affinis | SNCVADYSVLYNSTSFSTFKCYGVSAIKLNDLCFSNV |
| | coronavirus | YADSFVVKGDDVRQIAPGQTGVIADYNYKLPDDFMGCVLAWN |
| | LYRa11 | TRNIDATSSGNFNYKYRSLRHGKLRPFERDISNVPFSP |
| | S protein | DGKPCTPPAFNCYWPLNDYGFYTTNGIGYQPYRVVVLSFELL |
| | RBD | NAPATVCGPKLSTDLITNQCVNFNFNGLTGTGVLTPSL |
| | (Genbank: | KRFQPFQQFGRDFSDFTDSVRDPKTLEVL |
| | AHX37558) | |
| 56 | Rhinolophus | SNFRVSPSREVVRFPNITNLCPFGEVFNATTFPSVYAWERKRI |
| | affinis | SNCVADYSVLYNSTSFSTFKCYGVSAIKLNDLCFSNV |
| | coronavirus | YADSFVVKGDDVRQIAPGQTGVIADYNYKLPDDFMGCVLAWN |
| | LYRa3 | TRNIDATSSGNFHYKYRSLRHGKLRPFERDISNVPFSP |
| | S protein | DGKPCTPPAFNCYWPLNDYGFYTTNGIGYQPYRVVVLSFELL |
| | RBD | NAPATVCGPKLSTDLITNQCVNFNFNGLTGTGVLTPSS |
| | (Genbank: | KRFQPFQQFGRDVSDFTDSVRDPKTLEVL |
| | AHX37569) | |
| 57 | SARS | SNFRVVPSGDVVRFPNITNLCPFGEVFNATKFPSVYAWERKKI |
| | coronavirus | SNCVADYSVLYNSTFFSTFKCYGVSATKLNDLCFSNV |
| | Tor2 Spike | YADSFVVKGDDVRQIAPGQTGVIADYNYKLPDDFMGCVLAWN |
| | glycoprotein | TRNIDATSTGNYNYKYRYLRHGKLRPFERDISNVPFSP |
| | RBD | DGKPCTPPALNCYWPLNDYGFYTTTGIGYQPYRVVVLSFELL |
| | (Genbank: | NAPATVCGPKLSTDLIKNQCVNFNFNGLTGTGVLTPSS |
| | YP_009825051) | KRFQPFQQFGRDVSDFTDSVRDPKTSEIL |
| 58 | Bat SARS-like | SNFRVAPSKEVVRFPNITNLCPFGEVFNATTFPSVYAWERKRI |
| | coronavirus | SNCVADYSVLYNSTSFSTFKCYGVSATKLNDLCFSNV |
| | Rs3367 | YADSFVVKGDDVRQIAPGQTGVIADYNYKLPDDFTGCVLAWN |
| | S protein | TRNIDATQTGNYNYKYRSLRHGKLRPFERDISNVPFSP |
| | RBD | DGKPCTPPAFNCYWPLNDYGFYITNGIGYQPYRVVVLSFELLN |
| | (Genbank: | APATVCGPKLSTDLIKNQCVNFNFNGLTGTGVLTPSS |
| | AGZ48818) | KRFQPFQQFGRDVSDFTDSVRDPKTSEIL |
| 59 | SARS-like | SNFRVAPSKEVVRFPNITNLCPFGEVFNATTFPSVYAWERKRI |
| | coronavirus | SNCVADYSVLYNSTSFSTFKCYGVSATKLNDLCFSNV |
| | WIV16 | YADSFVVKGDDVRQIAPGQTGVIADYNYKLPDDFTGCVLAWN |
| | S protein | TRNIDATQTGNYNYKYRSLRHGKLRPFERDISNVPFSP |
| | RBD | DGKPCTPPAFNCYWPLNDYGFYITNGIGYQPYRVVVLSFELLN |
| | (Genbank: | APATVCGPKLSTDLIKNQCVNFNFNGLTGTGVLTPSS |
| | ALK02457) | KRFQPFQQFGRDVLDFTDSVRDPKTSEIL |
| 60 | Bat SARS-like | SNFRVAPSKEVVRFPNITNLCPFGEVFNATTFPSVYAWERKRI |
| | coronavirus | SNCVADYSVLYNSTSFSTFKCYGVSATKLNDLCFSNV |
| | Rs4231 | YADSFVVKGDDVRQIAPGQTGVIADYNYKLPDDFLGCVLAWN |
| | S protein | TNSKDSSTSGNYNYLYRWVRRSKLNPYERDLSNDIYSP |
| | RBD | GGQSCSAIGPNCYNPLRPYGFFTTAGVGHQPYRVVVLSFELL |
| | (Genbank: | NAPATVCGPKLSTDLIKNQCVNFNFNGLTGTGVLTSSS |
| | ATO98157) | KRFQPFQQFGRDVSDFTDSVRDPKTSEIL |
| 61 | Bat SARS-like | SNFRVAPSKEVVRFPNITNLCPFGEVFNATTFPSVYAWERKRI |
| | coronavirus | SNCVADYSVLYNSTSFSTFKCYGVSATKLNDLCFSNV |
| | RsSHC014 | YADSFVVKGDDVRQIAPGQTGVIADYNYKLPDDFLGCVLAWN |
| | S protein | TNSKDSSTSGNYNYLYRWVRRSKLNPYERDLSNDIYSP |
| | RBD | GGQSCSAVGPNCYNPLRPYGFFTTAGVGHQPYRVVVLSFELL |
| | (Genbank: | NAPATVCGPKLSTDLIKNQCVNFNFNGLTGTGVLTPSS |
| | AGZ48806) | KRFQPFQQFGRDVSDFTDSVRDPKTSEIL |
| 62 | Bat SARS-like | SNFRVAPSKEVVRFPNITNLCPFGEVFNATTFPSVYAWERKRI |
| | coronavirus | SNCVADYSILYNSTSFSTFKCYGVSATKLNDLCFSNV |
| | Rs4084 | YADSFVVKGDDVRQIAPGQTGVIADYNYKLPDDFLGCVLAWN |
| | S protein | TNSKDSSTSGNYNYLYRWVRRSKLNPYERDLSNDIYSP |
| | RBD | GGQSCSAVGPNCYNPLRPYGFFTTAGVGHQPYRVVVLSFELL |
| | (Genbank: | NAPATVCGPKLSTDLIKNQCVNFNFNGLTGTGVLTPSS |
| | ATO98132) | KRFQPFQQFGRDVSDFTDSVRDPKTSEIL |
| 63 | Bat SARS | SNFRVSPSTEVVRFPNITNLCPFGQVFNASNFPSVYAWERLRI |
| | coronavirus | SDCVADYAVLYNSSSSFSTFKCYGVSPTKLNDLCFSS |
| | BtKY72 | VYADYFVVKGDDVRQIAPAQTGVIADYNYKLPDDFTGCVLAW |
| | S protein | NTNSVDSKSGNNFYYRLFRHGKIKPYERDISNVLYNSA |
| | RBD | GGTCSSISQLGCYEPLKSYGFTPTVGVGYQPYRVVVLSFELLN |
| | (Genbank: | APATVCGPKKSTELVKNKCVNFNFNGLTGTGVLTSST |
| | APO40579) | KKFQPFQQFGRDVSDFTDSVRDPKTFEIL |
| 64 | SARS-like | SNFRVTPTTEVVRFPNITKLCPFDQVFNASSFPSVYAWERVRI |
| | coronavirus | TDCVANYAVLYNSSVSFSTFQCYGVSPTKLNDLCFSS |
| | BatCoV/BB9904/ | VYADYFIVKGDDVRQIAPSQTGVIADYNYKLPDDFTGCVIAWN |
| | BGR/2008 | TNALDSNKDFYYRLFRHGKIKPYGRDLSNIPYSPSGT |
| | S protein | CSTINNLNCFAPLKSYGFTQSSGISFQPYRVVVLSFELLNAPAT |
| | RBD | VCGPKQSTELVKDKCVNFNFNGLTGTGVLTSSSKKF |
| | (Genbank: | QPFQQFGRDVSDFTDSVRDPKTLEIL |
| | ALJ94036) | |
| 65 | Bat | SNFRVTPTTEVVRFPNITQLCPFNEVFNITSFPSVYAWERMRIT |
| | coronavirus | NCVADYSVLYNSSASFSTFQCYGVSPTKLNDLCFSS |
| | BM48- | VYADYFVVKGDDVRQIAPAQTGVIADYNYKLPDDFTGCVIAWN |
| | 31/BGR/2008 | TNSLDSSNEFFYRRFRHGKIKPYGRDLSNVLFNPSGG |
| | S protein | TCSAEGLNCYKPLASYGFTQSSGIGFQPYRVVVLSFELLNAPA |
| | RBD | TVCGPKQSTELVKNKCVNFNFNGLTGTGVLTNSTKKF |
| | (Genbank: | QPFQQFGRDVSDFTDSVRDPKTLEIL |
| | YP_003858584) | |
| 66 | Pangolin | SNFRVQPTESIVRFPNITNLCPFGEVFNATTFASVYAWNRKRIS |
| | coronavirus | NCVADYSVLYNSTSFSTFKCYGVSPTKLNDLCFTNV |
| | cDNA8-S | YADSFVVRGDEVRQIAPGQTGRIADYNYKLPDDFTGCVIAWN |
| | S protein | SNNLDSKVGGNYNYLYRLFRKSNLKPFERDISTEIYQA |
| | RBD | GSTPCNGVEGFNCYFPLQSYGFHPTNGVGYQPYRVVVLSFEL |
| | (Genbank: | LNAPATVCGPKQSTNLVKNKCVNFNFNGLTGTGVLTES |
| | QLR06864) | SKKFLPFQQFGRDIADTTDAVRDPQTLEIL |
| 67 | SARS-CoV2 | SNFRVQPTESIVRFPNITNLCPFGEVFNATRFASVYAWNRKRI |
| | S protein | SNCVADYSVLYNSASFSTFKCYGVSPTKLNDLCFTNV |
| | RBD | YADSFVIRGDEVRQIAPGQTGKIADYNYKLPDDFTGCVIAWNS |
| | (Genbank: | NNLDSKVGGNYNYLYRLFRKSNLKPFERDISTEIYQA |
| | YP_009724390) | GSTPCNGVEGFNCYFPLQSYGFQPTNGVGYQPYRVVVLSFE |
| | | LLHAPATVCGPKKSTNLVKNKCVNFNFNGLTGTGVLTES |
| | | NKKFLPFQQFGRDIADTTDAVRDPQTLEIL |
| 68 | SARS-CoV2 | SNFRVQPTESIVRFPNITNLCPFGEVFNATRFASVYAWNRKRI |
| | VOC May 01 | SNCVADYSFLYNSASFSTFKCYGVSLTKLNDLCFTNV |
| | RBD | YADSFVIRGDEVRQIAPGKTGNIADYNYKLPDDFTGCVIAWNS |
| | | KKLDSKVGGNYKYRFRLFRKSNLKPFERDISTEIYQA |
| | | GNKPCNGVKGFNCYSPLQPYGFQPTYGVGYQPYRVVVLSFE |
| | | LLHSPSTVCGPKKSTNLVKNKCVNFNFNGLIGTGVLTES |
| | | NKNFLPFQQFGRDIDDITDAVRDPQTLDIL |
| 69 | SARS-CoV2 | SNFRVQPTESIVRFPNITNLCPFGEVFNATRFASVYAWNRKRI |
| | VOC | SNCVADYSFLYNSASFSTFKCYGVSLTKLNDLCFTNV |
| | S protein | YADSFVIRGDEVRQIAPGQTGNIADYNYKLPDDFTGCVIAWNS |
| | RBD | KKLDSKVGGNYNYRFRLFRKSNLKPFERDISTEIYQA |
| | (Genbank: | GNKPCNGVKGFNCYFPLQPYGFQPTYGVGYQPYRVVVLSFE |
| | YP_009724390) | LLHSPSTVCGPKKSTNLVKNKCVNFNFNGLTGTGVLTES |
| | | NKKFLPFQQFGRDIDDITDAVRDPQTLEIL |
| 70 | Bat | SNFRVQPTDSIVRFPNITNLCPFGEVFNATTFASVYAWNRKRI |
| | coronavirus | SNCVADYSVLYNSTSFSTFKCYGVSPTKLNDLCFTNV |
| | RaTG13 | YADSFVITGDEVRQIAPGQTGKIADYNYKLPDDFTGCVIAWNS |
| | S protein | KHIDAKEGGNFNYLYRLFRKANLKPFERDISTEIYQA |
| | RBD | GSKPCNGQTGLNCYYPLYRYGFYPTDGVGHQPYRVVVLSFE |
| | (Genbank: | LLNAPATVCGPKKSTNLVKNKCVNFNFNGLTGTGVLTES |
| | QHR63300) | NKKFLPFQQFGRDIADTTDAVRDPQTLEIL |
| 71 | Pangolin | SNFRVQPTISIVRFPNITNLCPFGEVFNASKFASVYAWNRKRIS |
| | coronavirus | NCVADYSVLYNSTSFSTFKCYGVSPTKLNDLCFTNV |
| | GX-P1E | YADSFVVKGDEVRQIAPGQTGVIADYNYKLPDDFTGCVIAWNS |
| | S protein | VKQDALTGGNYLYRLFRKSKLKPFERDISTEIYQAGS |
| | RBD | TPCNGQVGLNCYYPLERYGFHPTTGVNYQPFRVVVLSFELLN |
| | (Genbank: | GPATVCGPKLSTTLVKDKCVNFNFNGLTGTGVLTTSKK |
| | QIA48623) | QFLPFQQFGRDISDTTDAVRDPQTLEIL |
| 72 | Pangolin | SNFRVQPTISIVRFPNITNLCPFGEVENASKFASVYAWNRKRIS |
| | coronavirus | NCVADYSVLYNSTSFSTFKCYGVSPTKLNDLCFTNV |
| | GX-P2V | YADSFVVKGDEVRQIAPGQTGVIADYNYKLPDDFTGCVIAWNS |
| | S protein | VKQDALTGGNYGYLYRLFRKSKLKPFERDISTEIYQA |
| | RBD | GSTPCNGQVGLNCYYPLERYGFHPTTGVNYQPFRVVVLSXEL |
| | (Genbank: | LNGPATVCGPKLSTTLVKDKCVNFNFNGLTGTGVLTTS |
| | QIQ54048) | KKQFLPFQQFGRDISDTTDAVRDPQTLEIL |
| 73 | Pangolin | SNFRVQPTISIVRFPNITNLCPFGEVFNASKFASVYAWNRKRIS |
| | coronavirus | NCVADYSVLYNSTSFSTFKCYGVSPTKLNDLCFTNV |
| | GX-P4L | YADSFVVKGDEVRQIAPGQTGVIADYNYKLPDDFTGCVIAWNS |
| | S protein | VKQDALTGGNYGYLYRLFRKSKLKPFERDISTEIYQA |
| | RBD | GSTPCNGQVGLNCYYPLERYGFHPTTGVNYQPFRVVVLSFEL |
| | (Genbank: | LNGPATVCGPKLSTTLVKDKCVNFNFNGLTGTGVLTTS |
| | QIA48614) | KKQFLPFQQFGRDISDTTDAVRDPQTLEIL |
| 74 | Pangolin | SNFRVQPTISIVRFPNITNLCPFGEVFNASKFASVYAWNRKRIS |
| | coronavirus | NCVADYSVLYNSTSFSTFKCYGVSPTKLNDLCFTNV |
| | GX-P5E | YADSFVVKGDEVRQIAPGQTGVIADYNYKLPDDFTGCVIAWNS |
| | S protein | VKQDALTGDNYGYLYRLFRKSKLKPFERDISTEIYQA |
| | RBD | GSTPCNGQVGLNCYYPLERYGFHPTTGVNYQPFRVVVLSFEL |
| | (Genbank: | LNGPATVCGPKLSTTLVKDKCVNFNFNGLTGTGVLTTS |
| | QIA48641) | KKQFLPFQQFGRDISDTTDAVRDPQTLEIL |
| 75 | Bat SARS | SNFRVQPQDTVVRFPNITNLCPFSEVFNATTFASVYAWNRKRI |
| | coronavirus | SNCVADYSVLYNSTSFSTFQCYGVSSTKLNDLCFTNV |
| | Rc-0319 | YADSFVVRGDEVRQIAPGQTGVIADYNYKLPDDFTGCVLAWN |
| | S protein | SRNQDASTSGNFNYYYRIWRSEKLRPFERDIAHYDYQV |
| | RBD | GTQFKSSLKNYGFYSSAGDSHQPYRVVVLSFELLNAPATVCG |
| | (Genbank: | PKQSTELIKNKCVNFNFNGLTGTGVLTDSNKKFQSFQQ |
| | BCG66627) | FGRDVSDFTDSVKDPKTLEVL |
| 76 | Bat SARS CoV | SNFRTSPTTQVVRFPNITNLCPFGEVFNATTFASVYAWNRRRI |
| | RShSTT182 | SNCVADYSVLYNTTSFSTFKCYGVSPTKLNDLCFTNV |
| | S protein | YADSFVVRGDEVRQIAPGQTGKIADYNYKLPDDFMGCVIAWN |
| | RBD | SISLDAGGSYYYRLFRKSVLKPFERDISTQLYQAGDKP |
| | (Genbank: | CSVEGPDCYYPLQSYYFQSTNGVGYQPYRVVVLSFELLNAPA |
| | RShSTT182/ | TVCGPKKSTHLVVNKCVNFNFNGLTGTGVLTSSTKKFL |
| | 2010\|EPI_ISL_ | PFQQFGRDVADTTNAVRDPQTLEVL |
| | 852604) | |
| 77 | SARS-related | MFIFILFLTLTSGSDLDRCTTFDDVQAPNYTQHTSSMRGVYYP |
| | coronavirus | DEIFRSDTLYLTQDLFLPFYSNVTGFHTINHTFGNPV |
| | Spike | IPFKDGIYFAATEKSNVVRGWVFGSTMNNKSQSVIIINNSTNVV |
| | glycoprotein | IRACNFELCDNPFFAVSKPMGTQTHTMIFDNAFNCT |
| | (Genbank: | FEYISDAFSLDVSEKSGNFKHLREFVFKNKDGFLYVYKGYQPI |
| | ABD72984) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 78 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | wtic-MB | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFFGYLKPTTFMLKYDENGTITDAVDCSQNPL |
| | ACZ72093) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 79 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Shanghai LY | IPFKDGIYFAATEKSNVVRGWVFGSTMNNKSQSVIIISNSTNVVI |
| | Spike | RACNFELCDNPFFAVSKPMGTQTHTMIFDNAFNCT |
| | glycoprotein | FEYISDAFSLDVSEKSGNFKHLREFVFKNKDGSLYVYKGYQPI |
| | (Genbank: | DVVRDLPSGFNTLKPIFKLPLGINITNFRAILTAFSP |
| | AAP82968) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTGNIDATSTGNYDYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLPFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAGIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 80 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | HKU-39849 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | ADC35483) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGIATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 81 | SARS-related | MLIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Spike | IPFKDGIYFAATEKSNVVRGWVFGSTMNNKSQSVIIINNSTNVV |
| | glycoprotein | IRACNFELCDNPFFAVSKPMGTQTHTMIFDNAFNCT |
| | (Genbank: | FEYISDAFSLDVSEKSGNFKHLREFVFKNKDGFLYVYKGYQPI |
| | ABD72982) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 82 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Tor2 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | AFR58672) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCXFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 83 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Tor2 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | AFR58714) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LXHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCXFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 84 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Tor2 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (Genbank: | DVVRDLPSGFNTLKPIFKLPLGINITNFRAILTAFSP |
| | YP_009825051) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCAFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 85 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | ShanghaiQXC1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | (Genbank: | FEYISDAFSLDVSEKSGNFKHLREFVFKNKDGFLYVYKGYQPI |
| | AAR86788) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCAFGGVSVITPGTNASSEVAVLYQDVNCT |
| | | NVSAAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKGELDKYFKNHTSPDVDLGDISGINASVVNIQK |
| | | EIDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 86 | SARS | MFIFLLFLTLTSGSDLDRCTTFDDVQAPNYTQHTSSMRGVYYP |
| | Coronavirus | DEIFRSDTLYLTQDLFLPFYSNVTGFHTINHTFGNPV |
| | CDC#200301157 | IPFKDGIYFAATEKSNVVRGWVFGSTMNNKSQSVIIINNSTNVV |
| | S protein | IRACNFELCDNPFFAVSKPMGTQTHTMIFDNAFNCT |
| | (Genbank: | FEYISDAFSLDVSEKSGNFKHLREFVFKNKDGFLYVYKGYQPI |
| | AAU81608) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPVGAGICASYHTVSLLRSTSQKSIVAYTMSLGA |
| | | DSSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 87 | SARS | MFIFLLFLTLTSGSDLDRCTTFDDVQAPNYTQHTSSMRGVYYP |
| | coronavirus P2 | DEIFRSDTLYLTQDLFLPFYSNVTGFHTINHTFGNPV |
| | Spike | IPFKDGIYFAATEKSNVVRGWVFGSTMNNKSQSVIIINNSTNVV |
| | glycoprotein | IRACNFELCDNPFFAVSKPMGTQTHTMIFDNAFNCT |
| | precursor | FEYISDAFSLDVSEKSGNFKHLREFVFKNKDGFLYVYKGYQPI |
| | (Genbank: | DVVRDLPSGFNTLKPIFKLPLGINITNFRAILTAFSP |
| | ACQ82725) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQGVNCT |
| | | DVSTAIHADQLTPAWRIYSTGNNVFQTQAGCLIGAE |
| | | HVDTSYECDIPVGAGICASYHTVSLLRSTSQKSIVAYTMSLGA |
| | | DSSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 88 | SARS | MFIFLLFLTLTSGSDLDRCTTFDDVQAPNYTQHTSSMRGVYYP |
| | coronavirus AS | DEIFRSDTLYLTQDLFLPFYSNVTGFHTINHTFGNPV |
| | Spike protein S | IPFKDGIYFAATEKSNVVRGWVFGSTMNNKSQSVIIINNSTNVV |
| | (Genbank: | IRACNFELCDNPFFAVSKPMGTQTHTMIFDNAFNCT |
| | AAQ94060) | FEYISDAFSLDVSEKSGNFKHLREFVFKNKDGFLYVYKGYQPI |
| | | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 89 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | AFM43867) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | YVDTSYECDIPIGAGICASYHTVSLLRSTSQKSIVAYTMSLGAD |
| | | SSIAYSNNTIAIPTNFSISIATEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 90 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (Genbank: | DVVRDLPSGFNTLKPIFKLPLGINITNFRAILTAFSP |
| | AGT21078) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRS |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | YVDTSYECDIPIGAGICASYHTVSLLRSTSQKSIVAYTMSLGAD |
| | | SSIAYSNNTIAIPTNFSISIATEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTVTAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 91 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Frankfurt 1 | IPFKDGIYFAATEKSNVVRGWVFGSTMNNKSQSVIIINNSTNVV |
| | Spike protein S | IRACNFELCDNPFFAVSKPMGTQTHTMIFDNAFNCT |
| | (Genbank: | FEYISDAFSLDVSEKSGNFKHLREFVFKNKDGFLYVYKGYQPI |
| | AAP33697) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDFGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 92 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Frankfurt1-v01 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (Genbank: | DVVRDLPSGFNTLKPIFKLPLGINITNFRAILTAFSP |
| | BAF42873) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | YVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLSDAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSFNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDFGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 93 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Frankfurt 1 | IPFKDGIYFAATEKSNVVRGWVFGSTMNNKSQSVIIINNSTNVV |
| | Spike protein | IRACNFELCDNPFFAVSKPMGTQTHTMIFDNAFNCT |
| | (Genbank: | FEYISDAFSLDVSEKSGNFKHLREFVFKNKDGFLYVYKGYQPI |
| | BAE93401) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRC |
| | | LRHGKLRPFERDISNVPFSPDGKPCTPPAFNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEFAVLYQDVNCT |
| | | DVSTAIHADQLTPAWRIYSTGNNVFQTQAGCLIGAE |
| | | YVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKSTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDFGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 94 | SARS-related | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Spike | IPFKDGIYFAATEKSNVVRGWVFGSTMNNKSQSVIIINNSTNVV |
| | glycoprotein | IRACNFELCDNPFFAVSKPMGTQTHTMIFDNAFNCT |
| | (Genbank: | FEYISDAFSLDVSEKSGNFKHLREFVFKNKDGFLYVYKGYQPI |
| | ABD72969) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYVDSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 95 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | BJ182-12 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein S | FEYISDAFSLDVSEKSGNFKHLREFVFKNKDGFLYVYKGYQPI |
| | (Genbank: | DVVRDLPSGFNTLKPIFKLPLGINITNFRAILTAFSP |
| | ACB69905) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFDGFNFSQILPDPLKPIKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINAPVVNIQKE |
| | | IDRLNEVAKNLNESLIDLRELGKYEQYIKWPWYVWL |
| | | GFIAGLIAIVMVTILLCCMTSCCSCLKGACSCGSCCKFDEDDSE |
| | | PVLKGVKLHYT |
| 96 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPI |
| | BJ182a | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein S | FEYISDAFSLDVSEKSGNFKHLREFVFKNKDGFLYVYKGYQPI |
| | (Genbank: | DVVRDLPSGFNTLKPIFKLPLGINITNFRAILTAFSP |
| | ACB69860) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFDGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWAFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLRELGKYEQYIKWPWYVWL |
| | | GFIAGLIAIVMVTILLCCMTSCCSCLKGACSCGSCCKFDEDDSE |
| | | PVLKGVKLHYT |
| 97 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | BJ182-8 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein S | FEYISDAFSLDVSEKSGNFKHLREFVFKNKDGFLYVYKGYQPI |
| | (Genbank: | DVVRDLPSGFNTLKPIFKLPLGINITNFRAILTAFSP |
| | ACB69894) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFDGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLRELGKYEQYIKWPWYVWL |
| | | GFIAGLIAIVMVTILLCCMTSCCSCLKGACSCGSCCKFDEDDSE |
| | | PVLKGVKLHYT |
| 98 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTLGNPV |
| | BJ182-4 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein S | FEYISDAFSLDVSEKSGNFKHLREFVFKNKDGFLYVYKGYQPI |
| | (Genbank: | DVVRDLPSGFNTLKPIFKLPLGINITNFRAILTAFSP |
| | ACB69883) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFDGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLRELGKYEQYIKWPWYVWL |
| | | GFIAGLIAIVMVTILLCCMTSCCSCLKGACSCGSCCKFDEDDSE |
| | | PVLKGVKLHYT |
| 99 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Sino1-11 | IPFKDGIYFAATEKSNVVRGWVFGSTMNNKSQSVIIINNSTNVV |
| | Spike protein | IRACNFELCDNPFFAVSKPMGTQTHTMIFDNAFNCT |
| | (Genbank: | FEYISDAFSLDVSEKSGNFKHLREFVFKNKDGFLYVYKGYQPI |
| | AAR23250) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRS |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 100 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | ACZ71797) | AELKCSVKSFEIDKGIYQTSNFRVVPSVDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNAFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMTKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINTSVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 101 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFPP |
| | (Genbank: | AQDIWGTSAAAYFVGYLKPTTFMLKYDENGTITDAVDCSQNPL |
| | ACZ72020) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMTKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 102 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | ACZ71976) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMTKTSVDC |
| | | NMYICGDSTECANLLLQYGSFCTQLNRALSGIAAEQDRNTREV |
| | | FAQVKQMYKTPTLKXFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEIAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 103 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | ACZ71961) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMTKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESFTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELNSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 104 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEXISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | ACZ72195) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMTKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 105 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | ACZ72108) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVVYTMSLGAD |
| | | SSIAYSNNTIAIPTNFSISITTEVMPVSMTKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 106 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | ACZ71826) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMTKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 107 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | ACZ72254) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMTKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKIHTSPDVDLGDISGINASVVNIQKEI |
| | | DRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 108 | SARS-related | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Spike | IPFKDGIYFAATEKSNVVRGWVFGSTMNNKSQSVIIINNSTNVV |
| | glycoprotein | IRACNFELCDNPFFAVSKPMGTQTHTMIFDNAFNCT |
| | (Genbank: | FEYISDAFSLDVSEKSGNFKHLREFVFKNKDGFLYVYKGYQPI |
| | ABD72972) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPNGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 109 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | MA15 ExoN1 | IPFKDGIYFAATEKSNVVRGWVFGSTMNNKSQSVIIINNSTNVV |
| | Spike | IRACNFELCDNPFFVVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | ACZ71991) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNHNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQMLPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 110 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | MA15 ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | ACZ72122) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNHNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQMLPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 111 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | MA15 ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | AEA10443) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNHNYKYRY |
| | | LXHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 112 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | MA15 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (Genbank: | AQDIWGTSAAAYFVGYLKPTTFMLKYDENGTITDAVDCSQNPL |
| | ACZ72035) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNHNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 113 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | MA15 ExoN1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | precursor | DVVRDLPSGFNTLKPIFKLPLGINITNFRAILTAFSP |
| | (GenBank: | AQDIWGTSAAAYFVGYLKPTTFMLKYDENGTITDAVDCSQNPL |
| | AEA10473) | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNHNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKXSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 114 | SARS-related | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Spike | IPFKDGIYFAATEKSNVVRGWVFGSTMNNKSQSVIIINNSTNVV |
| | glycoprotein | IRACNFELCDNPFIAVSKPMGTQTHTMIFDNAFNCT |
| | (GenBank: | FEYISDAFSLDVSEKSGNFKHLREFVFKNKDGFLYVYKGYQPI |
| | ABD73001) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIVIVMVTILLCCMTSCCSCLKGACSCGSCCKFDEDDSE |
| | | PVLKGVKLHYT |
| 115 | SARS-related | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Spike | IPFKDGIYFAATEKSNVVRGWVFGSTMNNKSQSVIIINNSTNVV |
| | glycoprotein | IRACNFELCDNPFFAVSKPMGTQTHTMIFDNAFNCT |
| | (GenBank: | FEYISDAFSLDVSEKSGNFKHLREFVFKNKDGFLYVYKGYQPI |
| | ABD72970) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIVIVMVTILLCCMTSCCSCLKGACSCGSCCKFDEDDSE |
| | | PVLKGVKLHYT |
| 116 | SARS-related | MFIFLLFLTLTNGSDLDRCTTFDDVQAPNYTQHTSSMRGVYYP |
| | coronavirus | DEIFRSDTLYLTQDLFLPFYSNVTGFHTINHTFGNPV |
| | Spike | IPFKDGIYFAATEKSNVVRGWVFGSTMNNKSQSVIIINNSTNVV |
| | glycoprotein | IRACNFELCDNPFFAVSKPMGTQTHTMIFDNAFNCT |
| | (GenBank: | FEYISDAFSLDVSEKSGNFKHLREFVFKNKDGFLYVYKGYQPI |
| | ABD72988) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIVIVMVTILLCCMTSCCSCLKGACSCGSCCKFDEDDSE |
| | | PVLKGVKLHYT |
| 117 | SARS-related | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | Spike | IPFKDGIYFAATEKSNVVRGWVFGSTMNNKSQSVIIINNSTNVV |
| | glycoprotein | IRACNFELCDNPFIAVSKPMGTQTHTMIFDNAFNCT |
| | (GenBank: | FEYISDAFSLDVSEKSGNFKHLREFVFKNKDGFLYVYKGYQPI |
| | ABD72985) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 118 | SARS-related | MFIFLLFLTLTRGSDLDRCTTFDDVQAPNYTQHTSSMRGVYYP |
| | coronavirus | DEIFRSDTLYLTQDLFLPFYSNVTGFHTINHTFGNPV |
| | Spike | IPFKDGIYFAATEKSNVVRGWVFGSTMNNKSQSVIIINNSTNVV |
| | glycoprotein | IRACNFELCDNPFIAVSKPMGTQTHTMIFDNAFNCT |
| | (GenBank: | FEYISDAFSLDVSEKSGNFKHLREFVFKNKDGFLYVYKGYQPI |
| | ABD72977) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 119 | SARS-related | MFIFLLFLTLTRGSDLDRCTTFDDVQAPNYTQHTSSMRGVYYP |
| | coronavirus | DEIFRSDTLYLTQDLFLPFYSNVTGFHTINHTFGNPV |
| | Spike | IPFKDGIYFAATEKSNVVRGWVFGSTMNNKSQSVIIINNSTNVV |
| | glycoprotein | IRACNFELCDNPFFAVSKPMGTQTHTMIFDNAFNCT |
| | (GenBank: | FEYISDAFSLDVSEKSGNFKHLREFVFKNKDGFLYVYKGYQPI |
| | ABD72979) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 120 | SARS-related | MFIFLLFLTLTRGSDLDRCTTFDDVQAPNYTQHTSSMRGVYYP |
| | coronavirus | DEIFRSDTLYLTQDLFLPFYSNVTGFHTINHTFGNPV |
| | Spike | IPFKDGIYFAATEKSNVVRGWVFGSTMNNKSQSVIIINNSTNVV |
| | glycoprotein | IRACNFELCDNPFFAVSKPMGTQTHTMIFDNAFNCT |
| | (GenBank: | FEYISDAFSLDVSEKSGNFKHLREFVFKNKDGFLYVYKGYQPI |
| | ABD72995) | DVVRDLPSGFNTLKPIFKLPLGINITNFRAILTAFSP |
| | | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVVDYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 121 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | BJ302 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAR07630) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 122 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | TJF | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAT76147) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRNTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGKCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 123 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | NS-1 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein S | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAR91586) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIMASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 124 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | BJ01 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein S | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAP30030) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 125 | SARS | MAIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | HHS-2004 | IPFKDGIYFAATEKSNVVRGWVFGSTMNNKSQSVIIINNSTNVV |
| | Spike protein | IRACNFELCDNPFFAVSKPMGTQTHTMIFDNAFNCT |
| | (GenBank: | FEYISDAFSLDVSEKSGNFKHLREFVFKNKDGFLYVYKGYQPI |
| | AAT74874) | DVVRDLPSGFNTLKPIFKLPLGINITNFRAILTAFSP |
| | | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LKHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DVLSRLDKVEAEVQIDRLITGRLQSLQTYVTQQLIRAAEIRASA |
| | | NLAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 126 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | WH20 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein S | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAX16192) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLMAIVMVTILLCCMTSCCSCLKGACSCGSCCKFDEDDS |
| | | EPVLKGVKLHYT |
| 127 | SARS | MFIFLLFFTLTSGSDLDRCTTFDDVQAPNYTQHTSSMRGVYYP |
| | coronavirus | DEIFRSDTLYLTQDLFLPFYSNVTGFHTINHTFGNPV |
| | BJ302 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAR07624) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVGFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 128 | SARS | MFIFLLFFTLTSGSDLDRCTTFDDVQAPNYTQHTSSMRGVYYP |
| | coronavirus | DEIFRSDTLYLTQDLFLPFYSNVTGFHTINHTFGNPV |
| | BJ302 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAR07626) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLSD |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPRSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVGFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 129 | SARS | MFIFLLFFTLTSGSDLDRCTTFDDVQAPNYTQHTSSMRGVYYP |
| | coronavirus | DEIFRSDTLYLTQDLFLPFYSNVTGFHTINHTFGNPV |
| | BJ302 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAR07629) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLSD |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTVDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVGFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 130 | SARS | MFIFLLFFTLTSGSDLDRCTTFDDVQAPNYTQHTSSMRGVYYP |
| | coronavirus | DEIFRSDTLYLTQDLFLPFYSNVTGFHTINHTFGNPV |
| | BJ302 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAR07625) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLSD |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPRSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTVDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVGFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 131 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | BJ302 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAR07627) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLSD |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPRSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTVDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVGFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 132 | SARS | MFIFLLFFTLTSGSDLDRCTTFDDVQAPNYTQHTSSMRGVYYP |
| | coronavirus | DEIFRSDTLYLTQDLFLPFYSNVTGFHTINHTFGNPV |
| | BJ302 | IPFKDGIYFAATEKSNVVRGWVFGSTMNNKSQSVIIINNSTNVV |
| | Spike | IRACIFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAR07628) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLSD |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPRSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTVDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVGFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 133 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | BJ302 | IPFKDGIYFAATEKSNVVRGWVFGSTMNNKSQSVIIINNSTNVV |
| | Spike | IRACIFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAR07631) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLSD |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPRSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTVDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVGFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVIVTILLCCMTSCCSCLKGACSCGSCCKFDEDDSE |
| | | PVLKGVKLHYT |
| 134 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | CS21 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | ABF68958) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLNG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 135 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFGNPV |
| | ES260 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | ABF68957) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLNG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCVTSCCSCLKGACSCGSCCKFDEDDSE |
| | | PVLKGVKLHYT |
| 136 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | CS24 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | ABF68959) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKKISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLND |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCAFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCVTSCCSCLKGACSCGSCCKFDEDDSE |
| | | PVLKGVKLHYT |
| 137 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | ES191 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | ABF68956) | AQDIWGTSAAAYFVGYLKPTTFMLKYDENGTITDAVDCSQNPL |
| | | AELKCSVKSFEIDKGIYQTSNFRVVPSGDVVRFPNIT |
| | | NLCPFGEVFNATKFPSVYAWERKRISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSIGITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCVTSCCSCLKGACSCGSCCKFDEDDSE |
| | | PVLKGVKLHYT |
| 138 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | WF188 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFPYVYKGYQPI |
| | (GenBank: | DVVRDLPSGFNTLKPIFKLPLGINITNFRAILTAFSP |
| | ABF68955) | AQDTWGTSAAAYFVGYLKPTTFMLKYDENGTITDAVDCSQNP |
| | | LAELKCSVKSFKIDKGIYQTSNFRVVPSGDVVRFPNIT |
| | | NLCPFGEVFNATKFPSVYAWERKRISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDTPIGAGICASYHTVSSLRSTSQKSIVAYTMSLGA |
| | | DSSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKYFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCVTSCCSCLKGACSCGSCCKFDEDDSE |
| | | PVLKGVKLHYT |
| 139 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | PC4-137 | IPFKDGIYFAATEKSNVVRGWVFGSTMNNKSQSVIIINNSTNVV |
| | Spike | IRACNFELCDNPFFVVSKPMGTRTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV49720) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLKG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHADQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFAQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 140 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | PC4-199 | IPFKDGIYFAATEKSNVVRGWVFGSTMNNKSQSVIIINNSTNVV |
| | Spike | IRACNFELCDNPFFVVSKPMGTRTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV49722) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFAQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 141 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | PC4-241 | IPFKDGIYFAATEKSNVVRGWVFGSTMNNKSQSVIIINNSTNVV |
| | Spike | IRACNFELCDNPFFVVSKPMGTRTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV49723) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLNG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 142 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | PC4-115 | IPFKDGIYFAATEKSNVVRGWVFGSTMNNKSQSVIIINNSTNVV |
| | Spike | IRACNFELCDNPFFVVSKPMGTRTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV49719) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLNG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 143 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDDPV |
| | GD03T0013 | IPFKDGIYFAATEKSNVVRGWVFGSTMNNKSQSVIIINNSTNVV |
| | Spike | IRACNFELCDNPFFVVSKPMGTRTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAS10463) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLNG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 144 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | B012 | IPFKDGIYFAATEKSNVVRGWVFGSTMNNKSQSVIIINNSTNVV |
| | Spike | IRACNFELCDNPFFVVSKPMGTRTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV97989) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLNG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNALTVL |
| | | TPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 145 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | civet020 | IPFKDGIYFAATEKSNVVRGWVFGSTMNNKSQSVIIINNSTNVV |
| | Spike | IRACNFELCDNPFFVVSKPMGTRTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAU04664) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 146 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | B033 | IPFKDGIYFAATEKSNVVRGWVFGSTMNNKSQSVIIINNSTNVV |
| | Spike | IRACNFELCDNPFFVVSKPMGTRTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV97992) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKHRY |
| | | LRHGKLRPFERDISNVPFSPDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 147 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | PC4-205 | IPFKDGIYFAATEKSNVVRGWVFGSTMNNKSQTVIIINNSTNVVI |
| | Spike | RACNFELCDNPFFVVSKPMGTRTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAU93319) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 148 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | C028 | IPFKDGIYFAATEKSNVVRGWVFGSTMNNKSQSVIIINNSTNVV |
| | Spike | IRACNFELCDNPFFVVSKPMGTRTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV98001) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTLAGCLIGAE |
| | | HVDTSYDCDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 149 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | A013 | IPFKDGIYFAATEKSNVVRGWVFGSTMNNKSQSVIIINNSTNVV |
| | Spike | IRACNFELCDNPFFVVSKPMGTRTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV97985) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYDNQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 150 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | civet014 | IPFKDGIYFAATEKSNVVRGWVFGSTMNNKSQSVIIINNSTNVV |
| | Spike | IRACNFELCDNPFFVVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAU04661) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLKG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 151 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | A001 | IPFKDGIYFAATEKSNVVRGWVFGSTMNNKSQSVIIINNSTNVV |
| | Spike | IRACNFELCDNPFFVVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV97984) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKXRY |
| | | LRHGKLRPFERDISNVPFSPXGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 152 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | C013 | IPFKDGIYFAATEKSNVVRGWVFGSTMNNKSQSVIIINNSTNVV |
| | Spike | IRACNFELCDNPFFVVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV97995) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKFRY |
| | | LRHGKLRPFERDISNVPFSPDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 153 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | C029 | IPFKDGIYFAATEKSNVVRGWVFGSTMDNKSQSVIIINNSTNVV |
| | Spike | IRACNFELCDNPFFVVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV98002) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 154 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | civet019 | IPFKDGIYFAATEKSNVVRGWVFGSTMNNKSQSVIIINNSTNVV |
| | Spike | IRACNFELCDNPFFVVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAU04662) | AQDTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSSDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFSGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 155 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | A021 | IPFKDGIYFAATEKSNVVRGWVFGSTMNNKSQSVIIINNSTNVV |
| | Spike | IRACNFELCDNPFFVVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV97986) | AQGTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLNG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTVIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFSGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 156 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | C018 | IPFKDGIYFAATEKSNVVRGWVFGSTMNNKSQSVIIINNSTNVV |
| | Spike | IRACNFELCDNPFFVVSKPMGTRTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFSP |
| | AAV97998) | AQGTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPFQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 157 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | B024 | IPFKDGIYFAATEKSNVVRGWVFGSTMNNKSQSVIIINNSTNVV |
| | Spike | IRACNFELCDNPFFVVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGIKITNFRAILTAFSP |
| | AAV97990) | AQGTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLNG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHADQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPFQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 158 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | C025 | IPFKDGIYFAATEKSNVVRGWVFGSTMNNKSQSVIIINNSTNVV |
| | Spike | IRACNFELCDNPFFVVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGIKITNFRAILTAFSP |
| | AAV98000) | AQGTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDSSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 159 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | civet010 | IPFKDGIYFAATEKSNVVRGWVFGSTMNNKSQSVIIINNSTNVV |
| | Spike | IRACNFELCDNPFFVVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGIKITNFRAILTAFSP |
| | AAU04649) | AQGTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSSDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 160 | Civet SARS | MFIFLLFLTLTSGSDLDRCTTFDDVQAPNYTQHTSSMRGVYYP |
| | CoV 007/2004 | DEIFRSDTLYLTQDLFLPFYSNVTGFHTINHTFDNPV |
| | Spike | IPFKDGIYFAATEKSNVVRGWVFGSTMNNKSQSVIIINNSTNVV |
| | glycoprotein | IRACNFELCDNPFFVVSKPMGTQTHTMIFDNAFNCT |
| | (GenBank: | FEYISDAFSLDVSEKSGNFKHLREFVFKNKDGFLYVYKGYQPI |
| | AAU04646) | DVVRDLPSGFNTLKPIFKLPLGIKITNFRAILTAFSP |
| | | AQGTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSSDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFSGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 161 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | A022 | IPFKDGIYFAATEKSNVVRGWVFGSTMNNKSQSVIIINNSTNVV |
| | Spike | IRACNFELCDNPFFVVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGIKITNFRAILTAFSP |
| | AAV91631) | AQGTWGTSAAAYFVGYLKPTTFMLKYDENGTITDAVDCSQNP |
| | | LAELKCSVKSFEIDKGIYQTSNFRVVPSGDVVRFPNIT |
| | | NLCPFGEVFNATKFPSVYAWERKRISNCVADYSVLYNSTSFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSSDGKPCTPPAPNCYWPLRG |
| | | YGFYTTSGIGYQPYRVVVLSFELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTLIHAEQLTPAWRIYSTGNNVFQTQAGCLIGAE |
| | | HVDTSYECDIPIGAGICASYHTVSSLRSTSQKSIVAYTMSLGAD |
| | | SSIAYSNNTIAIPTNFSISITTEVMPVSMAKTSVDC |
| | | NMYICGDSTECANLLLQYGSFCRQLNRALSGIAAEQDRNTRE |
| | | VFVQVKQMYKTPTLKDFGGFNFSQILPDPLKPTKRSFI |
| | | EDLLFNKVTLADAGFMKQYGQCLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFSGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 162 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | GD01 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein S | FEYISDAFSLDVSEKSGNFKHLREFVFKNKDGFLYVYKGYQPI |
| | (GenBank: | DVVRDLPSGFNTLKPIFKLPLGINITNFRAILTAFLP |
| | AAP51227) | AQDTWGTSAAAYFVGYLKPTTFMLKYDENGTITDAVDCSQNP |
| | | LAELKCSVKSFEIDKGIYQTSNFRVVPSRDVVRFPNIT |
| | | NLCPFGEVFNATKFPSVYAWERKRISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSYELLNAPATVCGPKLSTDLIKNQ |
| | | CVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKDFGGFNFSQILPDPLKSTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQKE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 163 | SARS | MFIFLLFLTLTSGSDLDRCTTFDDVQAPNYTQHTSSMRGVYYP |
| | coronavirus | DEIFRSDTLYLTQDLFLPFYSNVTGFHTINHTFDNPV |
| | GZ02 | IPFKDGIYFAATEKSNVVRGWVFGSTMNNKSQSVIIINNSTNVV |
| | Spike | IRACNFELCDNPFFAVSKPMGTQTHTMIFDNAFNCT |
| | glycoprotein | FEYISDAFSLDVSEKSGNFKHLREFVFKNKDGFLYVYKGYQPI |
| | (GenBank: | DVVRDLPSGFNTLKPIFKLPLGINITNFRAILTAFLP |
| | AAS00003) | AQDTWGTSAAAYFVGYLKPTTFMLKYDENGTITDAVDCSQNP |
| | | LAELKCSVKSFEIDKGIYQTSNFRVVPSRDVVRFPNIT |
| | | NLCPFGEVFNATKFPSVYAWERKRISNCVADYSVLYNSTFFST |
| | | FKCYGVSATKLNDLCFSNVYADSFVVKGDDVRQIAPG |
| | | QTGVIADYNYKLPDDFMGCVLAWNTRNIDATSTGNYNYKYRY |
| | | LRHGKLRPFERDISNVPFSPDGKPCTPPALNCYWPLND |
| | | YGFYTTTGIGYQPYRVVVLSFELLNAPATVCGPKLSTDLIKNQC |
| | | VNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAE |
| | | HVDTSYECDIPIGAGICASYHTVSLLRSTSQKSIVAYTMSLGAD |
| | | SSIAYSNNTIAIPTNFSISITTEVMPVSMAKTSVDC |
| | | NMYICGDSTECANLLLQYGSFCTQLNRALSGIAAEQDRNTREV |
| | | FAQVKQMYKTPTLKDFGGFNFSQILPDPLKPTKRSFI |
| | | EDLLFNKVTLADAGFMKQYGECLGDINARDLICAQKFNGLTVL |
| | | PPLLTDDMIAAYTAALVSGTATAGWTFGAGAALQIPF |
| | | AMQMAYRFNGIGVTQNVLYENQKQIANQFNKAISQIQESLTTT |
| | | STALGKLQDVVNQNAQALNTLVKQLSSNFGAISSVLN |
| | | DILSRLDKVEAEVQIDRLITGRLQSLQTYVTQQLIRAAEIRASAN |
| | | LAATKMSECVLGQSKRVDFCGKGYHLMSFPQAAPH |
| | | GVVFLHVTYVPSQERNFTTAPAICHEGKAYFPREGVFVFNGTS |
| | | WFITQRNFFSPQIITTDNTFVSGNCDVVIGIINNTVY |
| | | DPLQPELDSFKEELDKYFKNHTSPDVDLGDISGINASVVNIQEE |
| | | IDRLNEVAKNLNESLIDLQELGKYEQYIKWPWYVWL |
| | | GFIAGLIAIVMVTILLCCMTSCCSCLKGACSCGSCCKFDEDDSE |
| | | PVLKGVKLHYT |
| 164 | SARS-CoV | MFIFLFFLTLVSGSDLDRCTTFDDVQAPNYTQHTSSMRGVYYP |
| | spike protein | DEIFRSDTLYLTQDLFLPFYSNVTGFHTINHTFDNPVIPFKDGIY |
| | with foldon and | FAATEKSNVVRGWVFGSTMNNKSQSVIIINNSTNVVIRACNFE |
| | linker | LCDNPFFAVSKPMGTQTHTMIFDNAFNCTFEYISDAFSLDVSE |
| | | KSGNFKHLREFVFKNKDGFLYVYKGYQPIDVVRDLPSGFNTLK |
| | | PIFKLPLGINITNFRAILTAFSPAQDTWGTSAAAYFVGYLKPTTF |
| | | MLKYDENGTITDAVDCSQNPLAELKCSVKSFEIDKGIYQTSNF |
| | | RVVPSGDVVRFPNITNLCPFGEVFNATKFPSVYAWERKKISNC |
| | | VADYSVLYNSTFFSTFKCYGVSATKLNDLCFSNVYADSFVVKG |
| | | DDVRQIAPGQTGVIADYNYKLPDDFMGCVLAWNTRNIDATST |
| | | GNYNYKYRYLRHGKLRPFERDISNVPFSPDGKPCTPPALNCY |
| | | WPLNDYGFYTTTGIGYQPYRVVVLSFELLNAPATVCGPKLSTD |
| | | LIKNQCVNFNFNGLTGTGVLTPSSKRFQPFQQFGRDVSDFTD |
| | | SVRDPKTSEILDISPCSFGGVSVITPGTNASSEVAVLYQDVNCT |
| | | DVSTAIHADQLTPAWRIYSTGNNVFQTQAGCLIGAEHVDTSYE |
| | | CDIPIGAGICASYHTVSLLRSTSQKSIVAYTMSLGADSSIAYSNN |
| | | TIAIPTNFSISITTEVMPVSMAKTSVDCNMYICGDSTECANLLLQ |
| | | YGSFCTQLNRALSGIAAEQDRNTREVFAQVKQMYKTPTLKYF |
| | | GGFNFSQILPDPLKPTKRSFIEDLLFNKVTLADAGFMKQYGEC |
| | | LGDINARDLICAQKFNGLTVLPPLLTDDMIAAYTAALVSGTATA |
| | | GWTFGAGAALQIPFAMQMAYRFNGIGVTQNVLYENQKQIANQ |
| | | FNKAISQIQESLTTTSTALGKLQDVVNQNAQALNTLVKQLSSNF |
| | | GAISSVLNDILSRLDPPEAEVQIDRLITGRLQSLQTYVTQQLIRA |
| | | AEIRASANLAATKMSECVLGQSKRVDFCGKGYHLMSFPQAAP |
| | | HGVVFLHVTYVPSQERNFTTAPAICHEGKAYFPREGVFVFNG |
| | | TSWFITQRNFFSPQIITTDNTFVSGNCDVVIGIINNTVYDPLQPE |
| | | LDSFKEELDKYFKNHTSPDVDLGDISGINASVVNIQKEIDRLNE |
| | | VAKNLNESLIDLQELGKYEQYIKWP |
| | | LVPRGSGYIPEAPRDGQAYVRKDGEWVLLSTFLLEHHHHHHH |
| | | HHH |
| 165 | Artificial | MFIFLFFLTLVSGSTIEKCTTFDDVQPPNYTQFLSSHRGVYYPD |
| | Sarbecovirus | DIFRSDVLHLTQDHFLPFDSNVTGFITFGLNFDNPIIPFKDGIYF |
| | spike | AATEKSNVIRGWVFGSTMNNKSQSVIIMNNSTNVVIRACNFEL |
| | consensus 4 | CDNPFFAVSKPNNTQIPSYIFNNAFNCTFEYVSDDFNLDVGEK |
| | with foldon and | PGNFKHLREFVFKNKDGFLYVYSGYQPIDVASGLPSGFNALKP |
| | linker | IFKLPLGINITNFRTLLTAFPPRPDYWGTSAAAYFVGYLKPTTF |
| | | MLKYDENGTITDAVDCSQNPLAELKCSVKSFEIDKGIYQTSNF |
| | | RVAPSKEVVRFPNITNLCPFGEVFNATTFPSVYAWERKRISNC |
| | | VADYSVLYNSTSFSTFKCYGVSATKLNDLCFSNVYADSFVVKG |
| | | DDVRQIAPGQTGVIADYNYKLPDDFMGCVLAWNTRNIDATST |
| | | GNYNYKYRSLRHGKLRPFERDISNVPFSPDGKPCTPPAFNCY |
| | | WPLNDYGFFTTNGIGYQPYRVVVLSFELLNAPATVCGPKLSTD |
| | | LIKNQCVNFNFNGLTGTGVLTPSSKRFQPFQQFGRDVSDFTD |
| | | SVRDPKTSEILDISPCSFGGVSVITPGTNTSSEVAVLYQDVNCT |
| | | DVPVAIHADQLTPAWRVYSTGNNVFQTQAGCLIGAEHVDTSY |
| | | ECDIPIGAGICASYHTVSSLRSTSQKSIVAYTMSLGADSSIAYS |
| | | NNTIAIPTNFSISITTEVMPVSMAKTSVDCNMYICGDSTECANLL |
| | | LQYGSFCTQLNRALSGIAVEQDRNTREVFAQVKQMYKTPTLK |
| | | DFGGFNFSQILPDPLKPTKRSFIEDLLFNKVTLADAGFMKQYG |
| | | ECLGDINARDLICAQKFNGLTVLPPLLTDDMIAAYTAALVSGTA |
| | | TAGWTFGAGAALQIPFAMQMAYRFNGIGVTQNVLYENQKQIA |
| | | NQFNKAISQIQESLTTTSTALGKLQDVVNQNAQALNTLVKQLS |
| | | SNFGAISSVLNDILSRLDPPEAEVQIDRLITGRLQSLQTYVTQQ |
| | | LIRAAEIRASANLAATKMSECVLGQSKRVDFCGKGYHLMSFPQ |
| | | AAPHGVVFLHVTYVPSQERNFTTAPAICHEGKAYFPREGVFVF |
| | | NGTSWFITQRNFFSPQIITTDNTFVSGSCDVVIGIINNTVYDPLQ |
| | | PELDSFKEELDKYFKNHTSPDVDLGDISGINASVVNIQKEIDRL |
| | | NEVAKNLNESLIDLQELGKYEQYIKWPLVPRGSGYIPEAPRDG |
| | | QAYVRKDGEWVLLSTFLLEHHHHHHHHHH |

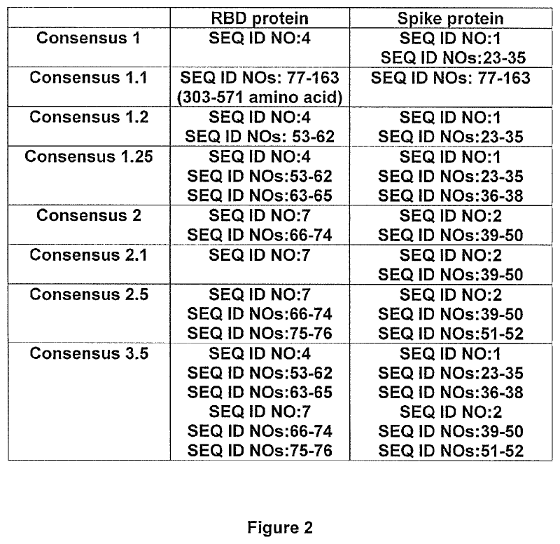

FIG. 2 illustrates a plurality of consensus sequences formed from various sequences listed above.

In various embodiments, the consensus sequence includes an amino acid sequence set forth in SEQ ID NO:4.

In various embodiments, the consensus sequence includes an amino acid sequence set forth in SEQ ID NOs: 77 to 163.

In various embodiments, the consensus sequence includes amino acid 303 to 571 of the amino acid sequence set forth in SEQ ID NOs: 77 to 163.

In various embodiments, the consensus sequence includes an amino acid sequence set forth in SEQ ID NO:4 and SEQ ID NOs:53 to 62 or any combination thereof.

In various embodiments, the consensus sequence includes an amino acid sequence set forth in SEQ ID NO:4 and SEQ ID NOs:53 to 62 or any combination thereof.

In various embodiments, the consensus sequence includes an amino acid sequence set forth in SEQ ID NO:4, SEQ ID NOs:53 to 62, and SEQ ID NOs:63 to 65 or any combination thereof.

In various embodiments, the consensus sequence includes an amino acid sequence set forth in SEQ ID NO:7 and SEQ ID NOs:66 to 74. or any combination thereof.

In various embodiments, the consensus sequence includes an amino acid sequence set forth in SEQ ID NO:7.

In various embodiments, the consensus sequence includes an amino acid sequence set forth in SEQ ID NO:7, SEQ ID NOs:66 to 74, and SEQ ID NOs:75 to 76 or any combination thereof.

In various embodiments, the consensus sequence includes an amino acid sequence set forth in SEQ ID NO:4, SEQ ID NOs:53 to 62, SEQ ID NOs:63 to 65, SEQ ID NOs:7, SEQ ID NOs:66-74, and SEQ ID NOs:75 to 76 or any combination thereof.

In various embodiments, the consensus sequence includes an amino acid sequence set forth in SEQ ID NO:1 and SEQ ID NOs:23-35 or any combination thereof.

In various embodiments, the consensus sequence includes an amino acid sequence set forth in SEQ ID NO:1, SEQ ID NOs:23-35, and SEQ ID NOs:36-38 or any combination thereof.

In various embodiments, the consensus sequence includes an amino acid sequence set forth in SEQ ID NO:2 and SEQ ID NOs:39 to 50 or any combination thereof.

In various embodiments, the consensus sequence includes an amino acid sequence set forth in SEQ ID NO:2, SEQ ID NOs:39-50 and SEQ ID NOs:51 to 52 or any combination thereof.

In various embodiments, the consensus sequence includes an amino acid sequence set forth in SEQ ID NO:1, SEQ ID NO:2, SEQ ID NOs:23-35, SEQ ID NOs:36-38, SEQ ID NOs:39-50 and SEQ ID NOs:51 to 52 or any combination thereof.

In various embodiments, the sarbecovirus is selected from the group comprising of SARS-CoV, SARS-CoV-2, SARS-CoV-2 B.1.1.7, SARS-CoV-2 B.1.351, SC2r-CoV RaTG13, SC2r-CoV GX-P5L, and SARS-CoV combined variants of concern (VOC).

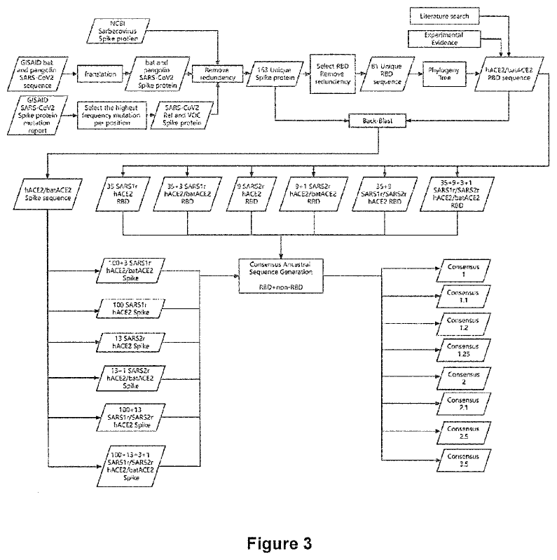

FIG. 3 illustrates the methodology used to generate the consensus sequences.

It is to be appreciated that the terms SARS1r hACE2/batACE2 Spike, SARS1r hACE2 Spike, SARS2r hACE2 Spike, SARS2r hACE2/batACE2 Spike, SARS1 r/SARS2r hACE2 Spike, or SARS1 r/SARS2r hACE2/batACE2 Spike are references to SEQ ID NOs. 23 to 52.

It is to be appreciated that the terms SARS1r hACE RBD, SARS1r hACE2/batACE2 RBD, SARS2r hACE2 RBD, SARS2r hACE/batACE2 RBD, SARS1 r/SARS2r hACE2 RBD, or SARS1 r/SARS2r hACE2/batACE2 RBD are references to SEQ ID NOs. 53 to 76.

It is to be appreciated that the terms SARS-CoV VOC or SARS-CoV-2 VOC are references to SEQ ID NOs: 77 to 163.

The method to generate consensus sequence from a genotype of sarbecovirus comprises: a) obtaining sarbecovirus spike protein sequences (except for human SARS-CoV2) directly from NCBI protein database and processing in R (v4.0.2) to filter complete protein sequences or by determining the corresponding nucleic acid using a genomic database such as GISAID genome and; b) retrieving and processing GISAID SARS-CoV2 spike protein mutation report in R (v4.0.2) with in-house script to calculate the highest frequency mutation per position using a microprocessor; c) producing the SARS-CoV2 reference and combined variants of concern (VOC) sequence (for example SARS-CoV-2 B.1.1.7, and/or SARS-CoV-2 B.1.351); d) after redundancy removal using CD-hit (v4.8.1), importing 163 unique spike and 81 unique RBD sequences into a microprocessor such as Geneious Prime (v2021.0) for further analysis; d) conducting protein alignment using MAFFT and plotting a phylogeny tree using PhyML; e) inferring the human ACE2, batACE2 and non-ACE2 lineages from the RBD phylogeny tree and validating with experimental evidence and literature search; and f) generating consensus sequences for RBD and non-RBD regions according to the highest frequency per amino acid position in the lineage and combining the initial sequences to establish 8 consensus sequences.

It is to be appreciated that the effect of the SARS survivors vaccinated with Pfizer-BioNTech mRNA SARS-CoV-2 vaccine BNT162b2 would be similar to using the consensus sequences generated in the present disclosure. Therefore, for the purposes of the following examples, they provide an example of how the amino acid constructs will work.

### Example 1: Human Serum Panel Studies were Conducted

The four serum panels included in this study were described as below. (1) SARS patient (n=11): these were sera collected from SARS survivors in Singapore at different time points (2003, 2012 and 2020) before the vaccination program started in February 2021; (2) COVID-19 patient (n=40): this group of sera was collected during 2020 as part of a national longitudinal study [19]. (3) Healthy-vaccinated (n=20): these were sera collected at day 14 after second dose (or 35 days after the first dose) of the Pfizer-BioNTech BNT162b2 mRNA vaccine. (4) SARS-vaccinated (n=9): sera taken from SARS survivors 21-62 days post first vaccination.

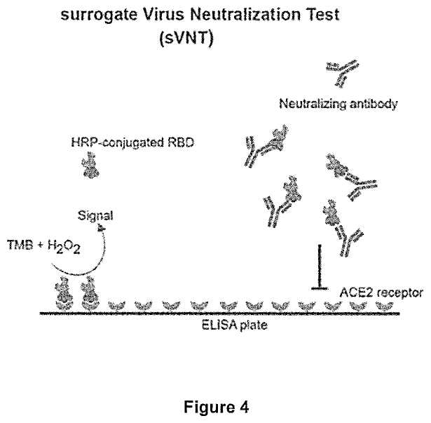

FIG. 4 shows a surrogate virus neutralization test (sVNT), which allowed rapid and multiplex determination of Nabs

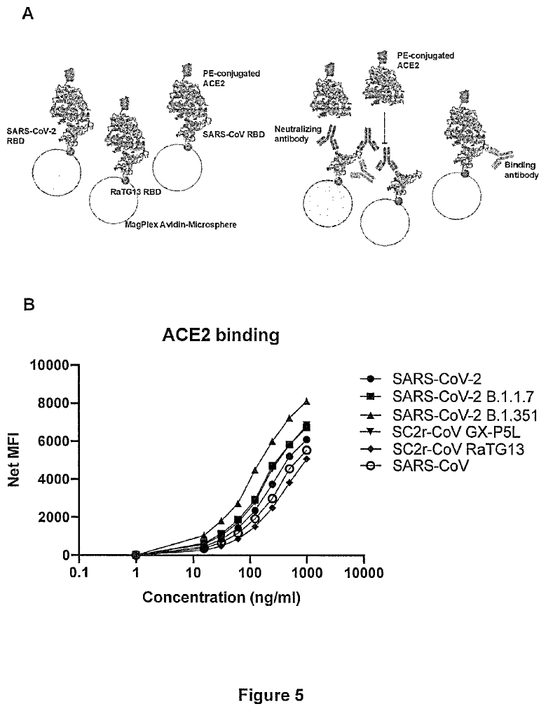

We have further improved the sVNT in two aspects: 1) we have immobilized the viral RBD on a solid phase (a magnetic bead) and used a PE-ACE2 to measure the virus-receptor binding (FIG. 5) which allowed multiplex detection of NAbs against different sarbecoviruses; 2) we have expanded the RBD proteins to six different viruses. As shown in FIG. 5, multiplex sVNT on the Luminex platform shows all six RBD proteins are able to bind the hACE2 receptor molecular as expected in the following order (from high to low affinity): SARS-CoV-2 B.1.351>SARS-CoV-2 B.1.1.7=SC2r-CoV GX-P5L (pangolin)>SARS-CoV-2>SARS-CoV>SC2r-CoV RaTG13 (bat).

Multiplex sVNT Based on RBD from Six Different Sarbecoviruses

AviTag-biotinylated RBDs were coated on the MagPlex Avidin microsphere (Luminex) at 5 μg/1 million beads. In multiplex sVNT, RBD-coated microspheres (600 beads/antigen) were pre-incubated with serum at a final 1:20 or greater for 1 h at 37° C. with 800 rpm agitation. After 1 h incubation, 50 μl of PE-conjugated hACE2 (GenScript, 1000 ng/ml) were added to the well and incubated for 30 min at 37° C. with agitation, followed by two PBS-1% BSA washes. The data were acquired using MAGPIX system.

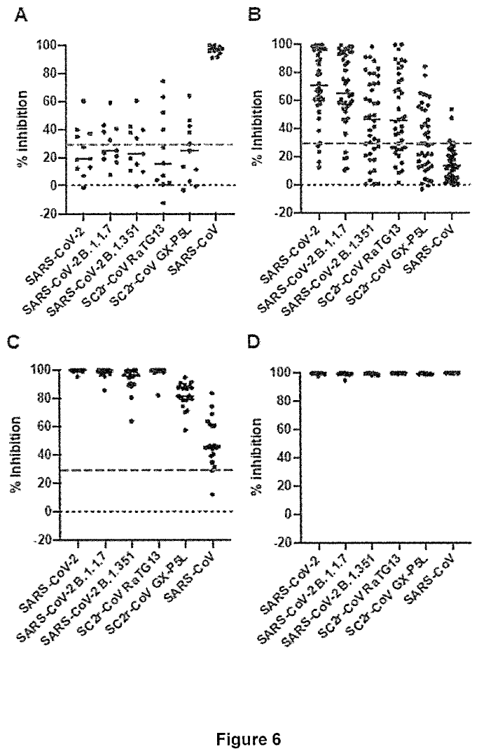

FIG. 6 shows multiplex sVNT on six different RBDs (from left to right: SARS-CoV-2 WT, UK, SA strains; Bat virus RaTG13; Pangolin virus GX-P5L; SARS-CoV). A). SARS patient. B) COVID-19 patient. C) Healthy-vaccinated. Serum samples were taken two weeks after second dose. D) SARS-vaccinated. All serum samples were tested at a single dilution of 1:20. A cut-off of 30% was set as previously determined.

The cross-NAb data demonstrated two highly important observations: The first observation is that vaccinated SARS survivors produced very high NAbs against all viruses studied (Panel D), even against the bat and pangolin viruses; and the second observation is that they neutralized the SARS-CoV-2 variants better than the naïve individuals who received the normal two doses (Panel C).

The SARS patients had minimal cross-NAbs to any of the other five viruses before vaccination (Panel A) whereas the COVID-19 patients had cross-NAbs against other viruses (all of them are SARS-CoV-2 related), they did not have much against the SARS-CoV (Panel B).

Using serial dilutions, the best performance of pan-sarbecovirus cross-neutralization by the SARS-vaccinated group have been illustrated.

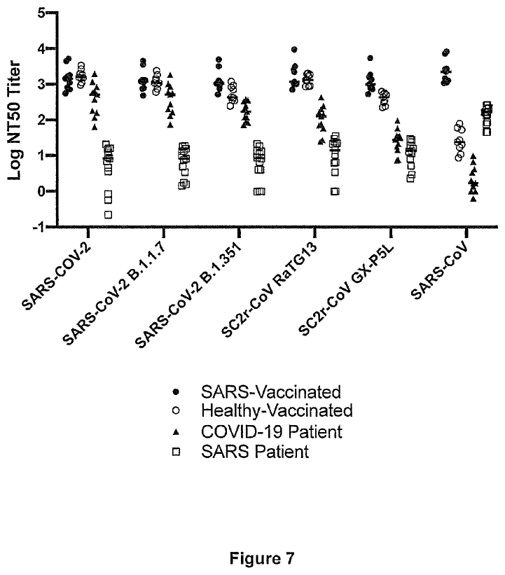

FIG. 7 shows titration of neutralizing antibody levels (NT50) in the different groups against the six sarbecoviruses. Serum samples were tested at dilutions from 1:20 to 1:20480 by a 4-fold serial titration. SAR-vaccinated shows highest log NT50 titer values against all six sarbecoviruses.

### Example 2: Mouse Studies

### Pan-Sarbecovirus mAb Inhibition Assay

RBD-coated microspheres (600 beads/antigen) were pre-incubated with 1:100 diluted serum for 1 h at 37° C. with agitation. Unbound antibodies were removed by two PBS-1% BSA washes. Pan-sarbecovirus mAbs (1000 ng/ml) were then added, followed by 1 h incubation at 37° C. with agitation followed by washing. The binding of the pan-sarbecovirus mAb on RBD was detected by PE-conjugated anti-mouse IgG antibodies. The data were acquired using MAGPIX system.

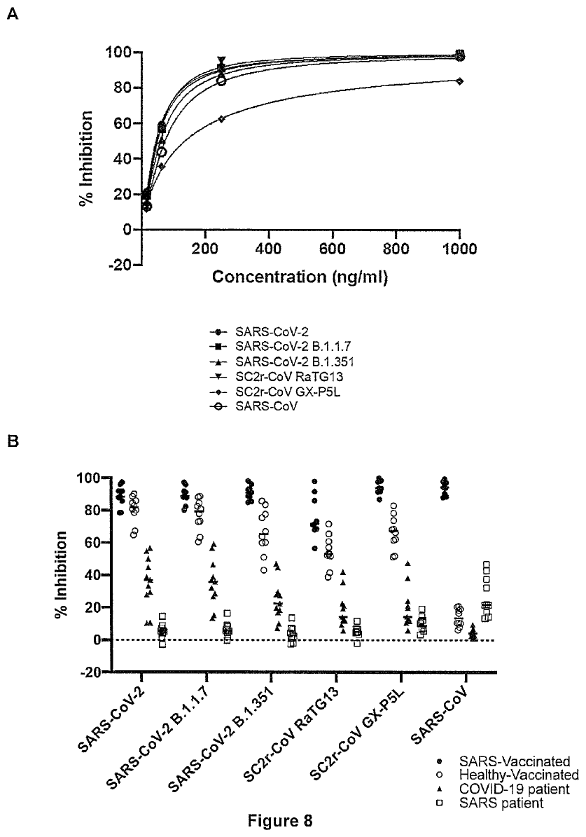

As shown in FIG. 8A, the mAb is able to neutralize all six viruses, albeit with slightly lower efficacy against GX-P5L. Using a blocking assay, same principle as the sVNT by replacing the PE-hACE2 with the mAb, we have determined the ability of the four different serum panels to block the mAb's ability in neutralization (FIG. 8B).

It should be appreciated that that the cross-neutralizing ability of the SARS-vaccinated group is the best among the four groups. Second, during natural infection (for either SARS and COVID-19), there is minimal activation of cross-neutralizing antibodies across the two lineages representing SC2r-CoVs and SARS-CoV. Third, although the mRNA vaccination boosted overall neutralizing ability against SCr2-CoVs, it had minimal impact on cross-neutralization against SARS-CoV.

### Example 3: Rabbit studies

Multiplex sVNT analysis using rabbit hyper immune sera targeting RBD of six different beta coronaviruses

The rabbit anti-RBD sera were produced by commercial contract with GenScript Biotech. The testing was conducted essentially the same as those described for the mouse studies. Rabbit sera were used by a 4-fold serial dilution starting at 1:20.

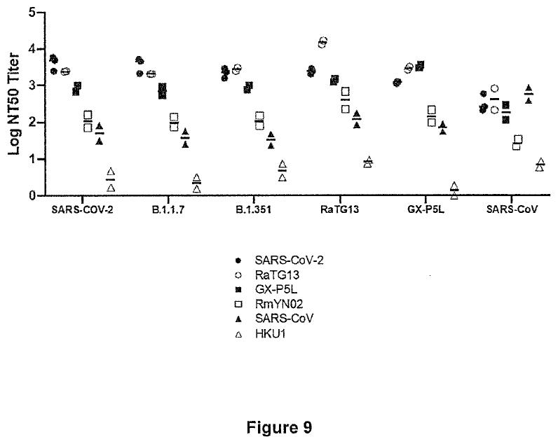

FIG. 9 shows neutralization patterns from rabbit hyper immune sera targeting different beta coronavirus RBD proteins. The data presented in FIG. 9 demonstrates that cross-neutralization is limited only to the strain/lineage level among the five SC2r-CoVs. There is no cross-neutralization between SC2r-CoV and SARS-CoV and the negative control HKU1 did not neutralize any virus/strain as shown. The data confirms the virus/strain-specific immunodominant antibody using rabbit hyper immune serum targeting specific virus/strain similar to the results as shown for human serum panel and mouse studies.

In should be appreciated that the amino acid constructs as mentioned herein above and in FIG. 2 may be used to form rabbit anti-amino acid construct sera or rabbit anti-consensus sequence sera to test against the six different sarbecoviruses used in the mouse studies above or the six different beta coronaviruses used in the Rabbit studies above.

### Example 4 Vaccination with Exemplary Protein

The protein listed in SEQ ID No. 165 (a modified version of SEQ ID No. 13, with foldon and linker) was administered in 25 μg with Sigma Adjuvant System (S6322) in 2 doses 21 days apart. Contemporaneously, a second dosage regime of a first dose of the protein listed in SEQ ID No. 165 (a modified version of SEQ ID No. 13, with foldon and linker) was administered with Sigma adjuvant followed by a second dose of the Sinovac's vaccine alone. The serum from these the dosed subjects was extracted for RBD challenge.

Using 20 RBD from different sarbecovirus were coated on separate microspheres. Each of the 20 RBD coated microspheres (600 beads/antigen) were pre-incubated with 1:100 diluted serum for 1 h at 37° C. with agitation. Unbound antibodies were removed by two PBS-1% BSA washes. The subject serum (1000 ng/ml) were then added, followed by 1 h incubation at 37° C. with agitation followed by washing. The binding of any antibodies formed in the subjects on the various RBD was detected by PE-conjugated anti-mouse IgG antibodies. The data were acquired using MAGPIX system.

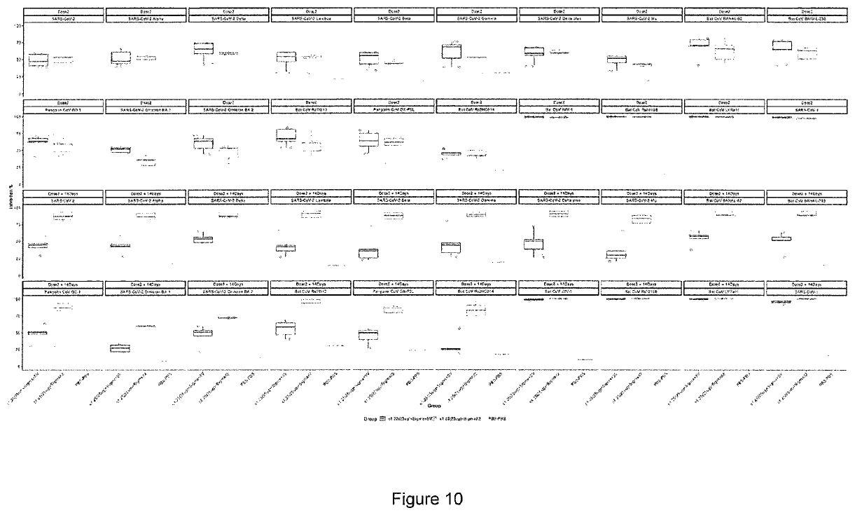

The two doses of the protein listed in SEQ ID No. 165 (a modified version of SEQ ID No. 13, with foldon and linker) administered with a Sigma adjuvant overall induced higher titre across all 20 RBD challenges carried out compared to the dosage regime of a first dose of the protein listed in SEQ ID No. 165 (a modified version of SEQ ID No. 13, with foldon and linker) administered with Sigma adjuvant followed by a second dose of the Sinovac's vaccine alone (See FIG. 10).

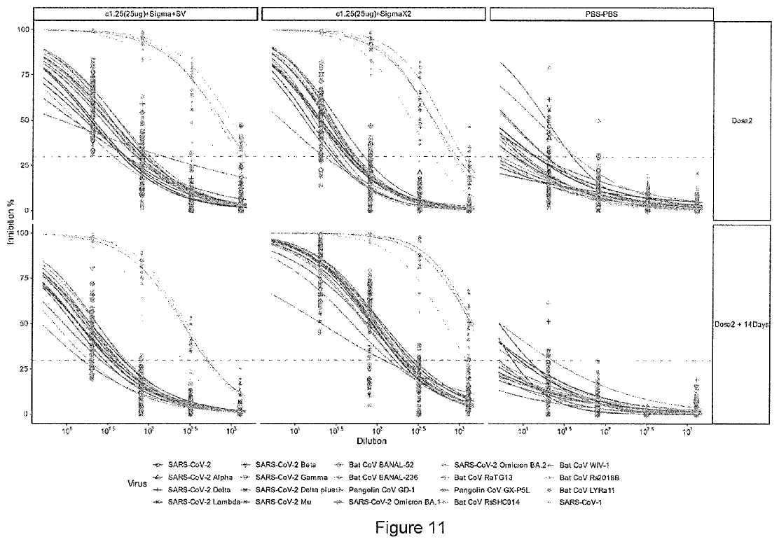

The same experiment was repeated using RBD from 20 different sarbecovirus and strains including the new variants of concern. As can be seen in FIG. 11, the two doses of the protein (25 μg) listed in SEQ ID No. 165 (a modified version of SEQ ID No. 13, with foldon and linker) administered with Sigma adjuvant overall induced higher titre across all 20 tested RBD compared to a first dose of the protein listed in SEQ ID No. 165 (a modified version of SEQ ID No. 13, with foldon and linker) administered with a Sigma adjuvant followed by a second dose of the Sinovac's vaccine alone.

### Example 5 Booster with Exemplary Protein

Separate dosage regimens were compared: the first being 3 doses of the Pfizer BioNTech vaccine; the second being 2 doses of the Pfizer BioNTech vaccine followed by a dose of 25 μg of the protein listed in SEQ ID No. 165 (a modified version of SEQ ID No. 13, with foldon and linker) administered with Sigma adjuvant; the third being 2 doses of the Pfizer BioNTech vaccine followed by a dose of 1 μg of the protein listed in SEQ ID No. 165 (a modified version of SEQ ID No. 13, with foldon and linker); the fourth being 2 doses of the Pfizer BioNTech vaccine followed by a dose of saline; and the final being 3 doses of saline.

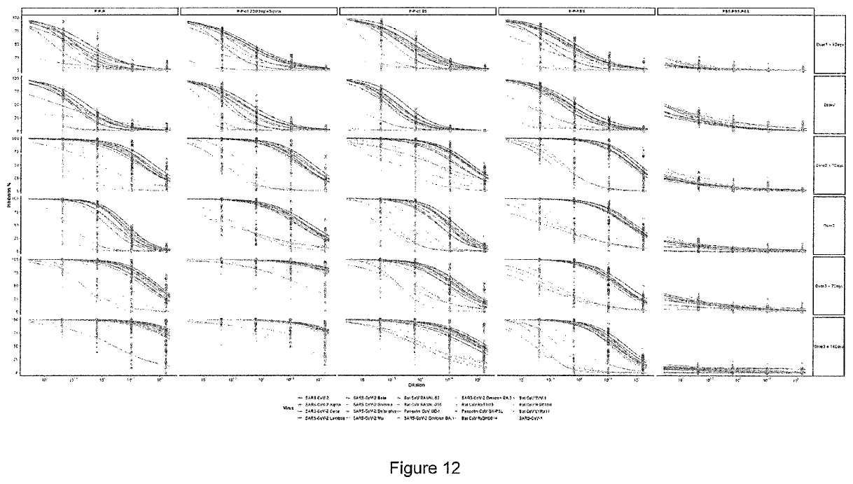

As can be seen in FIG. 12, 2 doses of the Pfizer BioNTech vaccine followed by a dose of the protein listed in SEQ ID No. 165 (a modified version of SEQ ID No. 13, with foldon and linker) administered either with or without the Sigma adjuvant all the clade 1 sarbecovirus were inhibited more than 3 doses with the Pfizer BioNTech vaccine both at 7 and 14 days after dose three.

Separate dosage regimens were compared: the first being 3 doses of the Moderna vaccine; the second being 2 doses of the Moderna vaccine followed by a dose of 25 μg of the protein listed in SEQ ID No. 165 (a modified version of SEQ ID No. 13, with foldon and linker) administered with Sigma adjuvant; the third being 2 doses of the Moderna vaccine followed by a dose of 1 μg of the protein listed in SEQ ID No. 165 (a modified version of SEQ ID No. 13, with foldon and linker); the fourth being 2 doses of the Moderna vaccine followed by a dose of saline; and the final being 3 doses of saline.

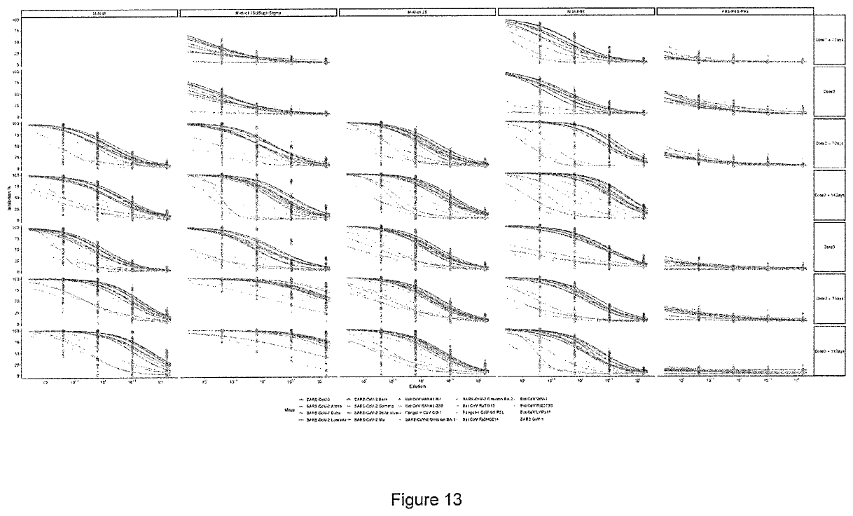

As can be seen in FIG. 13, 2 doses of the Pfizer BioNTech vaccine followed by a dose of the protein listed in SEQ ID No. 165 (a modified version of SEQ ID No. 13, with foldon and linker) administered either with or without the Sigma adjuvant all the clade 1 sarbecovirus were inhibited more than 3 doses with the Pfizer BioNTech vaccine both at 7 and 14 days after dose three.

Separate dosage regimens were compared: the first being 3 doses of the Sinovac vaccine; the second being 2 doses of the Sinovac vaccine followed by a dose of 25 μg of the protein listed in SEQ ID No. 164 (a modified version of SEQ ID No. 1 with foldon and linker sequence) administered with Sigma adjuvant; the third being 2 doses of the Sinovac vaccine followed by a dose of 25 μg of the protein listed in SEQ ID No. 165 (a modified version of SEQ ID No. 13 with foldon and linker sequence) administered with Sigma adjuvant; the fourth being 2 doses of the Sinovac vaccine followed by a dose of 1 μg of the protein listed in SEQ ID No. 165 (a modified version of SEQ ID No. 13 with foldon and linker sequence); and the final being 3 doses of saline.

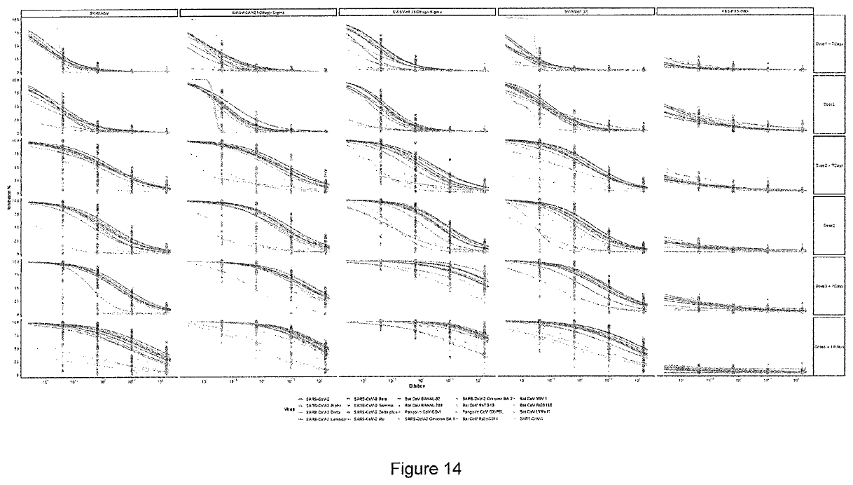

As can be seen in FIG. 14, 2 doses of the Sinovac vaccine followed by a dose of the protein listed in the protein listed in SEQ ID No. 165 (a modified version of SEQ ID No. 13 with foldon and linker sequence) administered either with or without the Sigma adjuvant all the clade 1 sarbecovirus were inhibited more than 3 doses with the Sinovac vaccine both at 7 and 14 days after dose three. 2 doses of the Sinovac vaccine followed by a dose of the protein listed in the protein listed in SEQ ID No. 165 (a modified version of SEQ ID No. 13 with foldon and linker sequence) administered with the Sigma adjuvant all the clade 1 and clade2 sarbecovirus were inhibited more than 2 doses of the Sinovac vaccine followed by a dose of the protein listed in the protein listed in SEQ ID No. 164 (a modified version of SEQ ID No. 1 with foldon and linker sequence) administered with the Sigma adjuvant both at 7 and 14 days after dose three. This result reflected the better booster effect of SEQ ID No. 13 than the natural SARS-CoV-1 shown as SEQ ID No.1 as the same modifications of foldon and linker were applied in SEQ ID No.165 and SEQ ID No.164.

It should be appreciated by the person skilled in the art that the above invention is not limited to the embodiment described. It is appreciable that modifications and improvements may be made without departing from the scope of the present invention.

It should be further appreciated by the person skilled in the art that one or more of the above modifications or improvements, not being mutually exclusive, may be further combined to form yet further embodiments of the present invention.

1. An amino acid construct comprising any one of an amino acid sequence selected from SEQ ID NOs: 10 to 22, 164 and 165, the construct having at least 90% sequence identity to at least two different sarbecovirus Spike proteins, or a fragment thereof.

2. The amino acid construct of claim 1, wherein the at least two different sarbecovirus Spike proteins comprise an amino acid sequence having at least 90% sequence identity to a sarbecovirus Spike protein, or an amino acid sequence having at least 90% sequence identity to a fragment of at least two different sarbecovirus Spike proteins.

3. The amino acid construct of claim 1, wherein the fragment comprises a receptor binding domain fragment of a sarbecovirus Spike protein.

4. The amino acid construct of any one of claims 1-3, wherein the amino acid sequence comprises a sequence set forth in SEQ ID NO: 10.

5. The amino acid construct of any one of claims 1-3, wherein the amino acid sequence comprises a sequence set forth in SEQ ID NO: 11.

6. The amino acid construct of any one of claims 1-3, wherein the amino acid sequence comprises a sequence set forth in SEQ ID NO: 12.

7. The amino acid construct of any one of claims 1-3, wherein the amino acid sequence comprises a sequence set forth in SEQ ID NO: 13.

8. The amino acid construct of any one of claims 1-3, wherein the amino acid sequence comprises a sequence set forth in SEQ ID NO: 14.

9. The amino acid construct of any one of claims 1-3, wherein the amino acid sequence comprises a sequence set forth in SEQ ID NO: 15.

10. The amino acid construct of any one of claims 1-3, wherein the amino acid sequence comprises a sequence set forth in SEQ ID NO: 16.

11. The amino acid construct of any one of claims 1-3, wherein the amino acid sequence comprises a sequence set forth in SEQ ID NO: 17

12. The amino acid construct of any one of claims 1-3, wherein the amino acid sequence comprises a sequence set forth in SEQ ID NO: 18.

13. The amino acid construct of any one of claims 1-3, wherein the amino acid sequence comprises a sequence set forth in SEQ ID NO: 19.

14. The amino acid construct of any one of claims 1-3, wherein the amino acid sequence comprises a sequence set forth in SEQ ID NO: 20.

15. The amino acid construct of any one of claims 1-3, wherein the amino acid sequence comprises a sequence set forth in SEQ ID NO: 21.

16. The amino acid construct of any one of claims 1-3, wherein the amino acid sequence comprises a sequence set forth in SEQ ID NO: 22.

17. The amino acid construct of any one of claims 4-16, wherein the amino acid sequence further comprises a foldon and a linker sequence.

18. The amino acid construct of claim 17, wherein the amino acid sequence comprises sequence set forth in SEQ ID NO: 165.

19. The amino acid construct according to any one of claims 1-18, comprising an oligomeric polypeptide.

20. The amino acid construct of claim 19, wherein the polypeptide is a fusion dimer.

21. A nucleic acid encoding the amino acid construct of any one of claims 1 to 18.

22. The nucleic acid according to claim 21, comprising a messenger ribonucleic acid (mRNA)

23. An immunogenic composition comprising the amino acid construct of any one of claims 1 to 19 or the nucleic acid of claim 21 or claim 22.

24. The immunogenic composition of claim 23, further comprising an adjuvant.

25. The immunogenic composition of claim 24, wherein the adjuvant is a Sigma Adjuvant System (S6533).

26. The immunogenic composition of claim 23, comprising at least two sarbecovirus virus antigen.

27. A viral vector comprising a nucleic acid of claim 21 or 22.

28. The viral vector of claim 27, wherein the vector is selected from a recombinant measles virus vector, a vesicular stomatitis virus (VSV) vector, a vaccinia virus vector or an adenovirus vector.

29. The amino acid construct according to any one of claims 1 to 19 for use in the treatment or prevention of sarbecovirus infections.

30. The immunogenic composition according to any one of claims 23 to 26 for use in the treatment and/or prevention of sarbecovirus infections.

31. The nucleic acid according to claim 21 or 22 for use in the treatment and/or prevention of sarbecovirus infections.

32. The viral vector according to claim 27 or 28 for use in the treatment and/or prevention of sarbecovirus infections.

33. Use of an amino acid construct according to any one of claims 1 to 19 in the manufacture of a medicament for the treatment and/or prevention of sarbecovirus infections.

34. Use of an immunogenic composition according to any one of claims 23 to 26 in the manufacture of a medicament for the treatment and/or prevention of sarbecovirus infections.

35. Use of a nucleic acid according to claim 21 or 22 in the manufacture of a medicament for the treatment and/or prevention of sarbecovirus infections.

36. Use of a viral vector according to claim 27 or 28 in the manufacture of a medicament for the treatment and/or prevention of sarbecovirus infections.

37. A method of treating and/or preventing an infection caused by sarbecovirus comprising administering a vaccine molecule comprising an amino acid construct according to any one of claims 1 to 19, an immunogenic composition according to any one of claims 23 to 26, a nucleic acid of claims 21 to 22, or a viral vector of claims 27 to 28 to a subject.

38. A method of making an amino acid construct for the treatment and/or prevention of sarbecovirus infections, comprising:
a. comparing amino acid sequences from at least two different sarbecovirus spike proteins or fragments thereof;
b. identifying identical amino acid in the sequences from the at least two different sarbecovirus spike proteins or fragments thereof;
c. removing any different amino acids from the sequences of the at least two different sarbecovirus spike proteins or fragments thereof to identify a unique amino acid sequence; and
d. forming the amino construct of the unique amino acid sequence wherein the amino construct has at least 90% sequence identity to the at least two different sarbecovirus Spike proteins, or a fragment thereof.

39. The method of claim 38, further comprising modifying the unique amino acid sequence with a foldon and a linker sequence.

40. The method of claim 38 or 39, further comprising identifying a fragment within the unique amino acid sequence that corresponds to a receptor binding domain of the at least two different sarbecovirus spike proteins to form a unique amino acid fragment sequence; and forming the amino construct of the combined unique amino acid fragment sequence with the unique amino acid sequence.

41. The method of any one of claims 38 to 40, wherein forming the amino construct in step d comprises forming a nucleic acid encoding the amino acid construct able to express the amino acid construct.

42. The method of claim 41, wherein the nucleic acid comprises a messenger ribonucleic acid (mRNA).

43. The method of any one of claims 38 to 42, wherein the amino acid construct or a nucleic acid encoding the amino acid construct able to express the amino acid construct is prepared as an immunogenic composition.

44. The method of claim 43, wherein the immunogenic composition comprises an adjuvant.

45. The method of claim 44, wherein the adjuvant is a Sigma Adjuvant system.

46. The method of claim 41 or 42, wherein the nucleic acid is prepared in a viral vector.

47. The method of claim 46, wherein the vector is selected from a recombinant measles virus vector, a vesicular stomatitis virus (VSV) vector, a vaccinia virus vector or an adenovirus vector.
